(19) United States
(12) Patent Application Publication
(10) Pub. No.: US 2023/0277452 A1
(43) Pub. Date: Sep. 7, 2023

(54) COMPOSITIONS, DEVICES AND METHODS FOR TREATING NASAL, OTIC AND OTHER TISSUE INFECTION AND/OR INFLAMMATION

(71) Applicant: Oticara, Inc., Austin, TX (US)

(72) Inventors: Michael McDonald Crowley, Austin, TX (US); Patrick Slater, Austin, TX (US); Christopher Marich, Austin, TX (US)

(73) Assignee: Oticara, Inc., Austin, TX (US)

(21) Appl. No.: 18/173,460

(22) Filed: Feb. 23, 2023

Related U.S. Application Data
(63) Continuation of application No. PCT/US2021/047769, filed on Aug. 26, 2021.
(60) Provisional application No. 63/070,812, filed on Aug. 26, 2020.

(51) Int. Cl. A61K 9/06; A61K 31/573; A61K 47/26; A61K 47/10; A61K 9/107; A61P 27/16; A61P 31/04; A61P 31/10; A61P 11/02

(52) U.S. Cl. CPC A61K 9/06; A61K 31/573; A61K 47/26; A61K 47/10; A61K 9/107; A61P 27/16; A61P 31/04; A61P 31/10; A61P 11/02

(57) ABSTRACT

Compositions, devices and methods are provided for treating diseases and conditions of the nasal, sinonasal, nasopharyngeal, otic and other tissues are provided.

### CROSS-REFERENCE TO RELATED APPLICATIONS

This application is a continuation of International Patent Application No. PCT/US2021/047769, filed Aug. 26, 2021, which claims the benefit of U.S. Provisional Application No. 63/070,812, filed Aug. 26, 2020, the disclosure of each of is hereby incorporated herein by reference in its entirety.

### BACKGROUND

A cream is a two phase emulsion prepared by combining two immiscible liquids, in which small globules of one liquid are dispersed uniformly throughout the other liquid. The liquid dispersed into small droplets is often referred to as the dispersed or internal phase. The other liquid is referred as the external phase or continuous phase. When oil is the dispersed phase, and an aqueous solution is the continuous phase, the system is designated as an oil-in-water (0/W) emulsion. Conversely, when water or an aqueous solution is the dispersed phase and oil or oleaginous material is the continuous phase, the system is designated as a water-in-oil (W/O) emulsion.

Creams are generally thermodynamically unstable, due to the large increase in surface energy that results from the combination of interfacial tension, the large surface area of the dispersed phase and the density differences of the two phases. Droplets of the internal phase can coalesce with a considerable reduction in surface free energy. Thus, creams tend to separate—the less dense phase rises and the denser phase falls. When exposed to heat, the homogenously distributed droplets begin to aggregate and ultimately coalesce into large globules and the cream becomes unstable, with phase separation typically occurring. The present disclosure addresses this issue by providing creams that do not separate when autoclaved or otherwise sterilized.

The nasal cavity, sinonasal cavity and nasopharynx are important components of the human respiratory system and can be affected by diseases or conditions requiring medical intervention. Proper and effective treatment of these diseases and conditions is necessary to promote the health of a patient and to avoid complications due to the disease or condition.

The current standard of care for diseases or conditions of these regions are saline nasal sprays or rinses, and corticosteroid, glucocorticoid, anticholergic, and antihistamine nasal sprays, which are generally low viscosity (1-10 cPs), water-based solutions or suspensions that are applied multiple times a day for an extended period of time. Simple nasal delivery methods such as drops, sprays, aerosols, nebulizers, and atomizers provide good nasal cavity contact but poor sinus delivery. Access to the sinus and low residence time from the low-viscosity liquids contribute to poor delivery. Additionally, while steroidal nasal sprays address the inflammation resulting from the condition, they may not address the underlying cause if it is an infection. These at-home therapies also require a high level of patient compliance for efficacy. There are also currently no FDA approved antifungals for nasal administration. Thus, there is a need for an efficacious product topically administered to the sinonasal or nasopharyngeal tissue for antifungal therapy.

With respect to the underlying cause of the condition, in some instances, a fungal and/or bacterial infection, the treatment involves irrigation of a water-based suspension of an antimicrobial or an antifungal in an in-clinic or hospital procedure that can include IV administration, and that may include anesthesia but most often are treated with nasal sprays at home by the patient, often in multiple daily doses. Alternatively or additionally, oral antibiotics and antifungals are prescribed. These treatments are often unsuccessful and patients continue to suffer from chronic infections and inflammation with no viable alternatives. Therefore, there is a need for a treatment option that addresses the deficiencies described above.

With respect to the poor delivery of steroids to the sinus mucosa, there is a need for a treatment option to overcome the deficiencies and inconvenience of liquid-based steroid delivery to the sinuses.

Otitis externa is a disease of the external ear that is characterized by inflammation of the meatal skin. Over 90% of cases of otitis externa can be traced to bacterial and/or fungal infections. In the incipient stage, symptoms of otitis include itching and pain in the ear canal, often accompanied by tenderness in the area around the external auditory meatus and pain when the ear lobe is pulled or when the jaw is moved. In the definitive stage, suppuration occurs in the ear canal, and may be accompanied by decreased auditory function. Treatment of otitis externa is complicated by the relative inaccessibility of the infected meatal skin, which makes it difficult to effectively apply a treatment to the affected area.

One of the most common types of otitis externa encountered by physicians is a type designated as “swimmer's ear”. Swimmer's ear has long been understood in the medical arts to be an infection of bacterial etymology and is treated accordingly. Hence, current medical practice for the treatment of swimmer's ear prescribes a multiple-dose, antibiotic ear drop regiment for the treatment of this condition. In some cases, these drops may include a small dosage of a steroid or an organic acid, such as acetic acid. Typically, the ear drops are applied to the infected ear two times a day for 10 days. Alternatively or additionally, oral antibiotics and pain medications are prescribed. This approach is consistent with standard medical practice in the treatment of bacterial infections, which seeks to eradicate the causal bacteria by (a) utilizing daily dosing so as to maintain a high level of an antibiotic in the patient's bloodstream, and (b) maintaining local contact over an extended period of time.

While an eardrop regimen may be an effective treatment for swimmer's ear in some cases, and offers the considerable convenience of being able to be administered by the patient, any interruption of the treatment which results in missed dosages or applications may result in failure to cure the disease. Moreover, the topical application of eardrops often results in inadequate physical contact with the surfaces to be treated, and even when proper contact is made, such contact may be of an insufficient duration to achieve the desired physiological effect. Moreover, current eardrop formulations are found to be ineffective in a significant number of cases, even if they are properly administered.

Often, the tympanic membrane is ruptured when an infection is present in the ear. As a result, ear drop regiments can enter the middle ear and inner ear through the rupture and expose those sensitive tissues and organs to the components of the ear drop regiment. This is problematic because many antimicrobials and inactive ingredients are ototoxic and cause damage to the middle ear, hair cells, the cochlea, the auditory nerve and sometimes the vestibular system resulting in permanent hearing loss. For example, aminoglycoside antibiotics, such as gentamicin, neomycin and tobramycin, are ototoxic. Inactive ingredients used in many topical drops, such as ethyl alcohol, acetic acid, chlorhexidine, and propylene glycol, are also ototoxic. Accordingly, there is a need for non-ototoxic compositions for treatment of ear disease.

Moreover, there is a need for sterile compositions to treat infections of the ear, sinus, and similar tissues. Non-sterile compositions can introduce additional pathogens to the diseased or infected tissue. Autoclaving is one of the common techniques used to sterilize pharmaceutical preparations. Creams are thermodynamically unstable. When exposed to heat conditions in an autoclave, a cream will generally flocculate, followed by coalescence, and ultimately phase separate back to oil and water.

Diseased and infected tissues are sensitive and often painful. In addition to treating the underlying infection, there is a need for treatments and compositions that address this sensitivity and maintain or improve patient comfort. Tonicity refers to the ability of a solution to cause a cell to gain or lose water. An isotonic solution causes no net gain or loss of water by the cell. A hypertonic solution causes water to leave the cell, while a hypotonic solution causes water to enter the cell. Medications that are far from isotonic can cause a feeling of pressure on the tissue and rupture cells. Thus, there is a need for treatments that are isotonic.

The effectiveness of an eardrop regimen, or of any other treatment requiring periodic application of a pharmaceutical composition, can often be optimized when practiced by a skilled physician. However, as a practical matter, many patients are unwilling to participate in treatments that require multiple visits to a hospital or healthcare provider. Consequently, a number of such patients avoid initial treatment or follow-up treatments, with the result that a readily curable condition of otitis externa matures into a more acute condition requiring serious medical intervention. A similar result may occur if there is any significant delay between the occurrence of the initial symptoms and subsequent treatment, as a result of, for example, a delay in scheduling an office visit. In this respect, it is notable that the growth rate of infecting organisms in diseased tissues is often exponential.

Alternative methods have been developed in the art for treating swimmer's ear and other types of otitis externa, frequently with an object of overcoming one or more of the aforementioned infirmities. Some of these treatments may be used in conjunction with an eardrop regimen. For example, one approach involves introducing into the infected area a ribbon gauze dressing soaked with antibacterial ear drops (the ear drops may contain a small dosage of a steroid) or with an astringent such as aluminum acetate solution. While such an approach may be very effective in some cases, it is not practical in many of the more acute instances of otitis externa, since contact between the inserted gauze and the inflamed meatal tissues can be extremely painful. Moreover, this approach cannot be administered by the patient, and hence requires the patient to visit a physician for the treatment.

There is thus a need in the art for a method for treating otitis externa which does not require multiple applications, which is amenable to treatment without delay, and which is effective in treating swimmer's ear and other types of otitis externa. There is further a need in the art for a method for treating otitis externa which is non-invasive and which effectively contacts the infected meatal skin. There is a need for sterile compositions that are not ototoxic. These and other needs are met by the devices and methodologies disclosed herein and hereinafter described.

### SUMMARY

The present disclosure provides compositions, devices and methods for treating diseases and conditions of the nasal, sinonasal, nasopharyngeal, otic and other tissues. More generally, the present disclosure provides compositions, devices and methods of treatment for delivery of therapeutically active ingredients to mucosal or other tissues by a cream that is isotonic and/or, in some instances, sterilizable such as by autoclaving without phase separation.

In some embodiments, a composition is provided that includes a tonicity agent and an emulsifier, where the composition is a cream and has an osmolality of about 270 mOsm/kg to about 360 mOsm/kg.

In some embodiments, methods are provided for treating diseases or conditions of the nasal, sinonasal, nasopharyngeal, otic or other tissues by administering a composition of the present disclosure to the tissue.

In some embodiments, a device is provided that has a length of tubing with a first end having an outer diameter and a tip at the second end with a largest diameter larger than the outer diameter of the first end and an arcuate shape as well as, optionally, a structural support element.

In some embodiments, the methods of treatment can use a device of the present disclosure to apply a composition, which can be a composition of the present disclosure to a nasal, sinonasal, nasopharyngeal or otic tissue to treat a disease or condition of the same.

In some embodiments, a kit is provided which includes a composition of the present disclosure and a device of the present disclosure.

### BRIEF DESCRIPTION OF THE DRAWINGS

For a more complete understanding of the present invention and the advantages thereof, reference is now made to the following description taken in conjunction with the accompanying drawings.

FIG. 1A depicts a workflow for an exemplary method of manufacturing a cream of the present disclosure.

FIG. 1B depicts a workflow for another exemplary method of manufacturing a cream of the present disclosure.

FIG. 2 depicts exemplary devices of the present disclosure.

FIG. 3A depicts an exemplary device of the present disclosure.

FIG. 3B depicts an exemplary device of the present disclosure.

FIG. 3C depicts an exemplary device of the present disclosure.

FIG. 3D depicts an exemplary device of the present disclosure.

FIG. 3E depicts an exemplary device of the present disclosure.

FIG. 4A depicts an exemplary device of the present disclosure.

FIG. 4B depicts an exemplary device of the present disclosure.

FIG. 4C depicts an exemplary device of the present disclosure.

FIG. 4D depicts an exemplary device of the present disclosure.

FIG. 5A depicts the results before and after autoclaving for a cream composition prepared in the Examples of the present disclosure.

FIG. 5B depicts the results before and after autoclaving for a cream composition prepared in the Examples of the present disclosure.

FIG. 5C depicts the results before and after autoclaving for a cream composition prepared in the Examples of the present disclosure.

FIG. 5D depicts the results before and after autoclaving for a cream composition prepared in the Examples of the present disclosure.

FIG. 5E depicts the results before and after autoclaving for a cream composition prepared in the Examples of the present disclosure.

FIG. 6 depicts a chart of osmolality versus amount of glycerin for cream compositions prepared in the Examples of the present disclosure.

FIG. 7A depicts a chart of the viscosity of cream compositions prepared in the Examples of the present disclosure versus shear rate.

FIG. 7B depicts a chart of the viscosity of cream compositions prepared in the Examples of the present disclosure versus autoclaving temperature.

FIG. 8 depicts beveled and non-beveled needle tips.

FIG. 9A depicts an injection setup of the Examples for the ototoxicity study.

FIG. 9B depicts an injection setup of the Examples for the ototoxicity study.

FIG. 10 depicts the ABR results for the ototoxicity study.

FIG. 11 depicts mean hair cell counts at different frequency regions for guinea pig ears treated with the test article or saline.

FIG. 12 depicts images of middle ears from guinea pigs treated with a composition of the present disclosure or saline.

FIG. 13A depicts mean sheep plasma concentrations of betamethasone-17-propionate and betamethasone.

FIG. 13B depicts mean and individual betamethasone-17-propionate plasma concentration plots.

FIG. 13C depicts mean and individual betamethasone plasma concentration plots.

FIG. 14A depicts an exemplary bent applicator device of the present disclosure.

FIG. 14B depicts an exemplary bent applicator device of the present disclosure.

### DETAILED DESCRIPTION

The present disclosure provides compositions, devices and methods for treating diseases and conditions of the nasal, sinonasal, nasopharyngeal, otic and other tissues. More generally, the present disclosure provides compositions, devices and methods of treatment for delivery of therapeutically active ingredients to mucosal or other tissues by a cream that is isotonic and, in some instances, sterilizable such as by autoclaving without phase separation.

### Definitions

As used herein, the singular forms “a”, “an” and “the” include plural referents unless the context clearly dictates otherwise.

The use of the term “or” in the claims and the present disclosure is used to mean “and/or” unless explicitly indicated to refer to alternatives only or the alternatives are mutually exclusive.

Use of the term “about”, when used with a numerical value, is intended to include +/−10%. By way of example but not limitation, if an amount is identified as about 1 mg, this would include 0.9 to 1.1 mg (plus or minus 10%).

As used herein, “antimicrobial” should be understood to include anti-microorganism such as antibacterial and antifungal. As used herein “agent with antimicrobial activity” and “antimicrobial agent” are synonymous.

As used herein, “effective amount” refers to an amount that is sufficient to bring about a desired pharmacologic and/or pharmacodynamic outcome.

For example, an effective amount for treatment is an amount that can reduce or eliminate symptoms and/or the pathology of an infection or disease. Another example is an effective amount to disrupt or eradicate the biofilms protecting a pathogen to effectively eliminate it.

The terms “patient,” “individual,” and “subject” are used interchangeably herein, and refer to a mammalian subject to be treated, with human patients being preferred. In some cases, the methods of the invention find use in experimental animals, in veterinary application, and in the development of animal models for disease and safety, including, but not limited to, rodents such as mice, rats, guinea pigs, and hamsters, as well as other animals including, but not limited to canines, sheep, felines, horses, and primates.

“Treatment” is an intervention performed with the intention of preventing the development or altering the pathology or symptoms of a disorder. Accordingly, “treatment” can refer to both therapeutic treatment and prophylactic or preventative measures. Those in need of treatment include those already with the disorder as well as those in which the disorder is to be prevented.

As used herein, the term “cream” means preparations containing one or more medicinal agents dissolved and/or dispersed in either an oil-in-water emulsion or water-in-oil emulsion. In avoidance of doubt, a “cream” does not include a “gel” which is a semisolid system consisting of dispersions of small or large molecules in an aqueous liquid vehicle rendered jelly like through addition of a gelling agent. Thus, the term “cream” does not include a thermoreversible gel, a thermoreversible polymer, or a copolymer of polyoxyethylene and polyoxypropylene. As used herein, a “cream” should be understood to have a viscosity of at least 25,000 cPs as measured using a Brookfield RVDVII+ at 1 rpm (shear rate) using Spindle 28.

It should also be understood that unless otherwise noted, reference to tonicity or osmolality is in units of mOsm/kg.

### Compositions

In some embodiments, a composition is provided that includes a tonicity agent and an emulsifier, where the composition is a cream and has an osmolality of about 270 mOsm/kg to about 360 mOsm/kg. In some embodiments, a composition is provided that is an autoclavable cream composition, wherein the composition is a cream, has an osmolality of about 270 mOsm/kg to about 360 mOsm/kg, and does not separate under autoclave conditions, such as at 110° C. for 10-30 minutes or 130° C. for 1-5 minutes. In the latter embodiments, the autoclavable cream composition can further include a tonicity agent and an emulsifier. By way of example, but not limitation, the composition of any of the foregoing embodiments can have an osmolality of between about 270 mOsm/kg and about 360 mOsm/kg, about 270 mOsm/kg and about 350 mOsm/kg, about 270 mOsm/kg and about 340 mOsm/kg, about 270 mOsm/kg and about 330 mOsm/kg, about 270 mOsm/kg and about 320 mOsm/kg, about 270 mOsm/kg and about 310 mOsm/kg, about 270 mOsm/kg and about 300 mOsm/kg, about 270 mOsm/kg and about 290 mOsm/kg, about 270 mOsm/kg and about 280 mOsm/kg, about 280 mOsm/kg and about 360 mOsm/kg, about 280 mOsm/kg and about 350 mOsm/kg, about 280 mOsm/kg and about 340 mOsm/kg, about 280 mOsm/kg and about 330 mOsm/kg, about 280 mOsm/kg and about 320 mOsm/kg, about 280 mOsm/kg and about 310 mOsm/kg, about 280 mOsm/kg and about 300 mOsm/kg, about 280 mOsm/kg and about 290 m/Osm/kg, about 290 mOsm/kg and about 360 mOsm/kg, about 290 mOsm/kg and about 350 mOsm/kg, about 290 mOsm/kg and about 340 mOsm/kg, about 290 mOsm/kg and about 330 mOsm/kg, about 290 mOsm/kg and about 320 mOsm/kg, about 290 mOsm/kg and about 310 mOsm/kg, about 290 mOsm/kg and about 300 mOsm/kg, about 300 mOsm/kg and about 360 mOsm/kg, about 300 mOsm/kg and about 350 mOsm/kg, about 300 mOsm/kg and about 340 mOsm/kg, about 300 mOsm/kg and about 330 mOsm/kg, about 300 mOsm/kg and about 320 mOsm/kg, about 300 mOsm/kg and about 310 mOsm/kg, about 310 mOsm/kg and about 360 mOsm/kg, about 310 mOsm/kg and about 350 mOsm/kg, about 310 mOsm/kg and about 340 mOsm/kg, about 310 mOsm/kg and about 330 mOsm/kg, about 310 mOsm/kg and about 320 mOsm/kg, about 320 mOsm/kg and about 360 mOsm/kg, about 320 mOsm/kg and about 350 mOsm/kg, about 320 mOsm/kg and about 340 mOsm/kg, about 320 mOsm/kg and about 330 mOsm/kg, about 330 mOsm/kg and about 360 mOsm/kg, about 330 mOsm/kg and about 350 mOsm/kg, about 330 mOsm/kg and about 340 mOsm/kg, about 340 mOsm/kg and about 360 mOsm/kg, about 340 mOsm/kg and about 350 mOsm/kg, about 350 mOsm/kg and about 360 mOsm/kg, about 270 mOsm/kg and about 310 mOsm/kg, about 280 mOsm/kg and about 320 mOsm/kg, about 290 mOsm/kg and about 310 mOsm/kg, about 310 mOsm/kg and about 360 mOsm/kg, about 270, 275, 280, 285, 290, 295, 300, 305, 310, 315, 320, 325, 330, 335, 340, 345, 350, 355, or 360 mOsm/kg and any range or value therebetween. It should be understood that these osmolalities apply to any composition within the scope of the present disclosure.

In some embodiments, the tonicity agent can be any agent suitable to produce a composition that is isotonic to blood. By way of example, but not limitation, the tonicity agent can be glycerin, propylene glycol, polyethylene glycol, butylene glycol, cyclomethicone, polydextrose, sodium hyaluronate, sodium lactate, sorbitol, trehalose, triacetin, xylitol, sodium chloride, potassium chloride or a combination thereof.

In any of the foregoing embodiments, the tonicity agent can be present in an amount of about 0.1% (w/w) to about 15% (w/w) based on the total weight of the composition. By way of example, but not limitation, the tonicity agent can be present in an amount of about 1% (w/w) to about 10% (w/w), about 1% (w/w) to about 5% (w/w), about 5% (w/w) to about 10% (w/w), about 5% (w/w) to about 15% (w/w), or about 10% (w/w) to about 15% (w/w) based on the total weight of the composition. By way of further example, but not limitation, the tonicity agent can be present in an amount of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.25% (w/w), 1.45% (w/w), 1.5% (w/w), 1.65% (w/w), 1.75% (w/w), 2% (w/w), 2.5% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), or about 15% (w/w) or any range or value therebetween based on the total weight of the composition.

In any of the foregoing embodiments, the composition can not include propylene glycol.

In any of the foregoing embodiments, the emulsifier can be any emulsifier suitable to produce to a cream composition. By way of example, but not limitation, the emulsifier can be polyoxyethylene sorbitan fatty acid ester, polyoxyethylene stearate, carboxymethylcellulose calcium, docusate sodium, an ethylene glycol stearate, glyceryl behenate, hydroxypropyl starch, lanolin, a lanolin alcohol, lauric acid, sodium laurate, lecithin, linoleic acid, medium-chain triglycerides, myristic acid, octyldodecanol, oleyl alcohol, palmitic acid, a phospholipid, a polyoxyethylene alkyl ether, a polyoxyethylene castor oil derivate, a polyoxylglcyeride, sodium lauryl sulfate, a sorbitan fatty acid ester, vitamin E polyethylene glycol succinate, cetyl alcohol, a nonionic emulsifying wax, hydrogenated castor oil, ceresin, cetostearyl alcohol, dextrin, paraffin, stearyl alcohol, an anionic emulsifying wax, a cetyl ester wax, microcrystalline wax, white wax, glyceryl monostearate, glyceryl monooleate, oleic acid, canola oil, castor oil, cholesterol, an ethylene glycol stearate, isopropyl myristate, isopropyl palmitate, mineral oil, a myristyl alcohol, safflower oil, triolein, xylitol, oleth-2, polysorbate 80, macrogol 15 hydroxystearate, or combinations thereof and those known to a person of skill in the art. By way of further example, in an embodiment, the emulsifier can include a combination of polysorbate 80, polyoxyl 40 stearate, cetyl alcohol, glyceryl monostearate and oleth-2. By way of still further example, the emulsifier can include a combination of polysorbate 80, polyoxyl 40 stearate, cetyl alcohol, glyceryl monostearate and Span 20 (sorbitan monolaurate). In some embodiments, the emulsifier can include a combination of a polyoxyethylene sorbitan fatty acid ester, a polyoxyethylene stearate, cetyl alcohol, glyceryl monostearate and a sorbitan fatty acid ester. By way of example, but not limitation, the polyoxyethylene sorbitan fatty acid ester can be present at about 0.1% (w/w) to about 15% (w/w), the polyoxyethylene stearate can be present at about 0.25% (w/w) to about 10% (w/w), cetyl alcohol can be present at about 0.25% (w/w) to about 10% (w/w), glyceryl monostearate can be present at about 0.1% (w/w) to about 10% (w/w), and the sorbitan fatty acid ester can be present at about 0.5% (w/w) to about 5% (w/w) based on the total weight of the composition, such as, the polyoxyethylene sorbitan fatty acid ester can be present at about 5% (w/w), the polyoxyethylene stearate can be present at about 1% (w/w), cetyl alcohol can be present at about 1% (w/w), glyceryl monostearate can be present at about 0.5% (w/w), and the sorbitan fatty acid ester can be present at about 3% (w/w) based on the total weight of the composition. By way of example, but not limitation, the polyoxyethylene sorbitan fatty acid ester can be polysorbate 90, the polyoxyethylene stearate can be polyoxyl 40 stearate and the sorbitan fatty acid ester can be oleth-2 or sorbitan monolaurate. In some embodiments, the emulsifier can include a sorbitan fatty acid ester such as sorbitan monolaurate.

In any of the foregoing embodiments, the emulsifier can be present in an amount sufficient to produce a cream composition. In any of the foregoing embodiments, the emulsifier can be present in an amount sufficient that the resulting cream composition can withstand autoclaving without separating into its component phases. By way of example, but not limitation, such autoclaving conditions can include 110° C. for 10 minutes, 110° C. for 19 minutes, 110° C. for 30 minutes, 130° C. for 1 minute, 130° C. for 3 minutes, or 130° C. for 5 minutes. By way of example, but not limitation, the emulsifier can be present in the composition in an amount from about 0.1% (w/w) to about 20% (w/w) based on the total weight of the composition. By way of example, but not limitation, the emulsifier can be present in an amount of about 0.1% (w/w) to about 20% (w/w), about 1% (w/w) to about 20% (w/w), about 5% (w/w) to about 20% (w/w), about 10% (w/w) to about 20% (w/w), about 15% (w/w) to about 20% (w/w), about 1% (w/w) to about 10% (w/w), about 1% (w/w) to about 5% (w/w), about 5% (w/w) to about 10% (w/w), about 5% (w/w) to about 15% (w/w), or about 10% (w/w) to about 15% (w/w) based on the total weight of the composition. By way of further example, but not limitation, the emulsifier can be present in an amount of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.25% (w/w), 1.5% (w/w), 1.75% (w/w), 2% (w/w), 2.5% (w/w), 3% (w/w), 4% (w/w), 5% (w/w), 6% (w/w), 7% (w/w), 8% (w/w), 9% (w/w), 10% (w/w), 10.5% (w/w), 11% (w/w), 12% (w/w), 13% (w/w), 14% (w/w), 15% (w/w), 16% (w/w), 17% (w/w), 18% (w/w), 19% (w/w), or 20% (w/w) or any range or value therebetween based on the total weight of the composition. By way of even further example, but not limitation, in some embodiments, the emulsifier can include polysorbate 80 in an amount of about 0.1% (w/w) to about 15% (w/w) based on the total weight of the composition, the polyoxyl 40 stearate can be present in the composition at about 0.25% (w/w) to about 10% (w/w) based on the total weight of the composition, the cetyl alcohol can be present in the composition at about 0.25% (w/w) to about 10% (w/w) based on the total weight of the composition, the glyceryl monostearate can present in the composition at about 0.1% (w/w) to about 5% (w/w) based on the total weight of the composition, and the oleth-2 can be present in the composition at about 0.5% (w/w) to about 10% (w/w) based on the total weight of the composition. For example, a composition of the present disclosure can include polysorbate 80 at about 5% (w/w) based on the total weight of the composition, polyoxyl 40 stearate at about 1% (w/w) based on the total weight of the composition, cetyl alcohol at about 1% (w/w) based on the total weight of the composition, glyceryl monostearate at about 0.5% (w/w) based on the total weight of the composition, and oleth-2 at about 3% (w/w) based on the total weight of the composition. By way of still further example, but not limitation, in some embodiments, the emulsifier can include polysorbate 80 in an amount of about 0.1% (w/w) to about 15% (w/w) based on the total weight of the composition, the polyoxyl 40 stearate can be present in the composition at about 0.25% (w/w) to about 10% (w/w) based on the total weight of the composition, the cetyl alcohol can be present in the composition at about 0.25% (w/w) to about 10% (w/w) based on the total weight of the composition, the glyceryl monostearate can present in the composition at about 0.1% (w/w) to about 5% (w/w) based on the total weight of the composition, and the oleth-2 can be present in the composition at about 0.5% (w/w) to about 10% (w/w) based on the total weight of the composition. For example, a composition of the present disclosure can include polysorbate 80 at about 5% (w/w) based on the total weight of the composition, polyoxyl 40 stearate at about 1% (w/w) based on the total weight of the composition, cetyl alcohol at about 1% (w/w) based on the total weight of the composition, glyceryl monostearate at about 0.5% (w/w) based on the total weight of the composition, and Span 20 at about 3% (w/w) based on the total weight of the composition

In any of the foregoing embodiments, the composition can further include a viscosity modifying agent. In any of the foregoing embodiments, the viscosity modifying agent can be any pharmaceutically acceptable viscosity modifying agent. By way of example, but not limitation, the viscosity modifying agent can be a carbomer, such as Carbopol 940 or Carbopol 980, acacia, calcium alginate, sodium alginate, carrageenan, chitosan, hypromellose, hydroxypropyl cellulose, methyl cellulose, polycarbophil, poly(methyl vinyl ether/maleic anhydride, xanthan, or a combinations thereof and those known to a person of skill in the art.

In any of the foregoing embodiments, the viscosity modifying agent can be present in the composition in an amount sufficient to maintain the composition as a cream. In any of the foregoing embodiments, the viscosity modifying agent can be present in an amount sufficient that the resulting cream composition can withstand autoclaving without separating into its component phases. By way of example, but not limitation, such autoclaving conditions can include 110° C. for 10 minutes, 110° C. for 30 minutes, 130° C. for 1 minute, 130° C. for 3 minutes, or 130° C. for 5 minutes. By way of example, but not limitation, the viscosity modifying agent can be present in the composition in an amount from about 0.1% (w/w) to about 10% (w/w) based on the total weight of the composition. By way of further example, but not limitation, the viscosity modifying agent can be present in the composition in an amount of about 0.1% to about 10% (w/w), about 0.1% (w/w) to about 5% (w/w), about 0.1% (w/w) to about 3% (w/w), about 0.1% (w/w) to about 2% (w/w), about 0.1% to about 1% (w/w), about 1% (w/w) to about 10% (w/w), about 1% (w/w) to about 5% (w/w), about 1% (w/w) to about 4% (w/w), about 1% (w/w) to about 3% (w/w), about 1% (w/w) to about 2% (w/w), about 2% (w/w) to about 10% (w/w) about 2% (w/w) to about 5% (w/w), about 2% (w/w) to about 4% (w/w), about 2% (w/w) to about 3% (w/w), about 3% (w/w) to about 10% (w/w), about 3% (w/w) to about 5% (w/w), about 3% (w/w) to about 4% (w/w), about 4% (w/w) to about 10% (w/w), about 4% (w/w) to about 5% (w/w), about 5% (w/w) to about 10% (w/w), about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.5% (w/w), 2% (w/w), 2.5% (w/w), 3% (w/w), 3.5% (w/w), 4% (w/w), 4.5% (w/w), 5% (w/w), 6% (w/w), 7% (w/w), 8% (w/w), 9% (w/w), or 10% (w/w) or any range or value therebetween based on the total weight of the composition. By way of further example, but not limitation, where the viscosity modifying agent is hydroxypropyl methylcellulose, the hydroxypropyl methylcellulose can be present in the composition in an amount of about 2% (w/w) to about 5% (w/w) based on the total weight of the composition. By way of still further example, but not limitation, the viscosity modifying agent can be a carbomer, such as carbomer 980, and be present in the composition at an amount of about 0.6% (w/w).

In any of the foregoing embodiments, the composition can further include a pH-modifying agent. In any of the foregoing embodiments, the pH-modifying agent can be added in an amount sufficient to result in the composition having a pH of between about 3.5 and about 8, preferably about 4 and about 7, more preferably about 5 and about 6. By way of example, but not limitation, the pH-modifying agent can be present in amount sufficient to adjust the pH of the composition to about 3.5 to about 8, about 4 to about 7, about 5 to about 7, about 5 to about 6, about 6 to about 7, about 4 to about 6, about 4 to about 5, or about 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 and any range or value therebetween. By way of example, but not limitation, the pH-modifying agent can be sodium hydroxide, potassium hydroxide, boric acid, sodium borate, triethanolamine, or a combination thereof and those known to one of skill in the art.

In any of the foregoing embodiments, the pH-modifying agent can be present in the composition in an amount sufficient to minimize chemical degradation of pharmaceutically active compounds in the formulation, such a steroid or agent with antimicrobial agent. By way of example, but not limitation, the pH-modifying agent can be present in the composition in an amount from about 0.005% (w/w) to about 0.15% (w/w) based on the total weight of the composition. By way of further example, but not limitation, the pH-modifying agent can be present in the composition in an amount of about 0.005% (w/w) to about 0.1% (w/w), about 0.005% (w/w) to about 0.05% (w/w), about 0.05% (w/w) to about 0.1% (w/w), about 0.05% (w/w) to about 0.15% (w/w), or about 0.1% (w/w) to about 0.15% (w/w) based on the total weight of the composition. By way of further example, but not limitation, the pH-modifying agent can be present in an amount of about 0.005% (w/w), 0.006% (w/w), 0.007% (w/w), 0.008% (w/w), 0.009% (w/w), 0.01% (w/w), 0.0125% (w/w), 0.015% (w/w), 0.0175% (w/w), 0.02% (w/w), 0.025% (w/w), 0.03% (w/w), 0.04% (w/w), 0.05% (w/w), 0.06% (w/w), 0.07% (w/w), 0.08% (w/w), 0.09% (w/w), 0.1% (w/w), 0.11% (w/w), 0.12% (w/w), 0.13% (w/w), 0.14% (w/w), or about 0.15% (w/w) or any range or value therebetween based on the total weight of the composition. It should be understood that the pH-modifying agent can be added a neat preparation or as a diluted solution to the composition, including in the methods of the present disclosure. Thus, where a diluted solution, such as a 1% NaOH solution is used, the amount of the solution added would need to be sufficient to add the pH-modifying agent in the appropriate amount.

In any of the foregoing embodiments, the composition can further include a tonicity modifier. In any of the foregoing embodiments, the tonicity modifier can be present in an amount sufficient to yield the desired tonicity, i.e. osmolality of the composition. In any of the foregoing embodiments, the tonicity modifier can be benzyl alcohol, benzalkonium chloride, chlorhexidine, phenylethyl alcohol, sodium metabisulfite, methyl paraben, propyl paraben, or a combination thereof. In any of the foregoing embodiments, the tonicity modifier can be present in the composition in an amount of about 0.5% (w/w) to about 15% (w/w) based on the total weight of the composition. By way of example, but not limitation, the tonicity modifier can be present in an amount of about 0.5% (w/w) to about 10% (w/w), about 0.5% (w/w) to about 5% (w/w), about 5% (w/w) to about 10% (w/w), about 5% (w/w) to about 15% (w/w), or about 10% (w/w) to about 15% (w/w) based on the total weight of the composition. By way of further example, but not limitation, the tonicity modifier can be present in an amount of about 0.5% (w/w), 0.6% (w/w), 0.7% (w/w), 0.8% (w/w), 0.9% (w/w), 1% (w/w), 1.25% (w/w), 1.5% (w/w), 1.75% (w/w), 2% (w/w), 2.5% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), or about 15% (w/w) or any range or value therebetween based on the total weight of the composition.

In any of the foregoing embodiments, the composition can further include an emollient. In any of the foregoing embodiments, the emollient can be petrolatum, mineral oil, light mineral oil, paraffin, a petrolatum or paraffin alcohol, white petrolatum, or a combination thereof and those known to one of skill in the art. In any of the foregoing embodiments the emollient can be present in the composition in an amount of about 4% (w/w) to about 30% (w/w) based on the total weight of the composition. By way of example, but not limitation, the emollient can be present in the composition in an amount of about 4% (w/w) to about 10% (w/w), about 4% (w/w) to about 20% (w/w), about 10% (w/w) to about 20% (w/w), about 10% (w/w) to about 30% (w/w), or about 20% (w/w) to about 30% (w/w) based on the total weight of the composition. By way of further example, but not limitation, the emollient can be present in an amount of about 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w) based on the total weight of the composition.

In any of the foregoing embodiments, the composition can further comprise a vehicle. Any pharmaceutically acceptable aqueous vehicle can be used. By way of example, but not limitation, the vehicle can be water.

In any of the foregoing embodiments, the composition can further comprise a steroid. Various corticosteroids, glucocorticoids or combinations thereof can be used in the compositions and methods of the present disclosure. By way of example but not limitation, corticosteroids that can be used in the compositions and methods of the present disclosure include cortisone, cortisol, hydrocortisone, methylprednisolone, prednisolone, prednisone, triamcinolone, betamethasone, ciclesonide, dexamethasone, 21-acetoxypregnenolone, alclometasone, algestone, amcinonide, beclomethasone, budesonide, chloroprednisone, clobetasol, clobetasone, clocortolone, cloprednol, corticosterone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednival, prednylidene, rimexolone, tixocortol, triamcinolone, triamcinolone acetonide, triamcinolone benetonide, triamcinolone hexacetonide, and the like. Esters, derivatives and salts, including hydrates and hydrogen chloride salts of corticosteroids can also be used in the compositions and methods of the present disclosure. For example, betamethasone is frequently administered as betamethasone dipropionate (which has the chemical name 9-Fluoro-11β,17,21-trihydroxy-16β-methylpregna-1,4-diene-3,20-dione 17,21-dipropionate, which has an empirical formula of C₂₈H₃₇FO₇, and which has a molecular weight of 504.59 g/mol), and the dosage given for betamethasone in Table 1 below is based on this particular salt. It should be understood that any pharmaceutically acceptable of a steroid can be used in the compositions and methods of the present disclosure.

In any of the foregoing embodiments, the steroid can be present in the composition in an “effective amount.” The amount of steroid in compositions of the present disclosure can vary according to the desired dose to be delivered based on patient status, patient sensitivity, the route of administration the biological half-life of the steroid, the patient's age, systemic factors, and other factors. In addition, the state of the infection or disease and its susceptibility to the steroid can also be considered. One of skill in the art can determine an appropriate dosage, including determining an “effective amount” of the composition to apply.

In any of the foregoing embodiments, the steroid can be present in the composition at about 0.01% (w/w) to about 15% (w/w) based on the total weight of the composition. By way of example, but not limitation, the steroid can be present in the composition at about 0.01% (w/w) to about 15% (w/w), about 0.01% (w/w) to about 10.5% (w/w), about 0.01% (w/w) to about 8.5% (w/w), about 0.01% (w/w) to about 3.5% (w/w), about 0.01% (w/w) to about 2% (w/w), 0.01% (w/w) to about 1.7% (w/w), about 0.01% (w/w) to about 0.03% (w/w), about 1% (w/w) to about 10.5% (w/w), about 0.8% (w/w) to about 8% (w/w), about 1.7% (w/w) to about 17% (w/w), about 0.2% (w/w) to about 2% (w/w), about 0.025% (w/w) to about 0.25% (w/w), about 0.03% (w/w) to about 0.3% (w/w), about 0.01% (w/w), about 0.02% (w/w), about 0.03% (w/w), 0.0322% (w/w), 0.05% (w/w), 0.0644% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2% (w/w), 2.25% (w/w), 2.5% (w/w), 2.75% (w/w), 3% (w/w), 3.25% (w/w), 3.5% (w/w), 3.75% (w/w), 4% (w/w), 4.25% (w/w), 4.5% % (w/w), 4.75% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5% (w/w), 10% (w/w), 10.5% (w/w), 11% (w/w), 12% (w/w), 13% (w/w), 14% (w/w), or 15% (w/w) and any range or value therebetween.

In any of the foregoing embodiments, where the steroid is betamethasone dipropionate, the steroid can be present in the composition, by way of example, but not limitation, at about 0.01% (w/w) to about 1.0% (w/w), more preferably 0.03% (w/w) to about 0.6% (w/w), based on the total weight of the composition. By way of example, but not limitation, the steroid can be present in the composition in an amount of about 0.01% (w/w) to about 1.0% (w/w), about 0.01% (w/w) to about 0.5% (w/w), 0.02% (w/w) to about 0.8% (w/w), 0.03% (w/w) to about 0.7% (w/w), 0.0322% (w/w) to about 0.0644% (w/w), 0.04% (w/w) to about 0.6% (w/w), 0.05% to about 0.5% (w/w), about 0.01% (w/w) to about 0.1% (w/w), about 0.01% (w/w) to about 0.09% (w/w), about 0.01% (w/w) to about 0.08% (w/w), about 0.01% (w/w) to about 0.07% (w/w), about 0.1% (w/w) to about 0.06% (w/w), about 0.01% (wow) to about 0.05% (w/w), about 0.01% (w/w) to about 0.04% (w/w), about 0.01% (w/w) to about 0.03% (w/w), about 0.01% (w/w) to about 0.02% (w/w), about 0.1% (w/w) to about 1.0% (w/w), about 0.1% (w/w) to about 0.2% (w/w), about 0.2% (w/w) to about 1.0% (w/w), about 0.3% (w/w) to about 1.0% (w/w), about 0.4% (w/w) to about 1.0% (w/w), about 0.5% (w/w) to about 1.0% (w/w), about 0.1% (w/w) to about 0.5% (w/w), about 0.5% (w/w) to about 1.0% (w/w), about 0.6% (w/w) to about 1.0% (w/w), about 0.7% (w/w) to about 1.0% (w/w), about 0.8% (w/w) to about 1.0% (w/w), or about 0.9% (w/w) to about 1.0% (w/w) based on the total weight of the composition. By way of further example, but not limitation, the steroid can be present in the composition in an amount of about 0.015% (w/w), 0.02% (w/w), 0.025% (w/w), 0.03% (w/w), 0.0322% (w/w), 0.035% (w/w), 0.04% (w/w), 0.045% (w/w), 0.05% (w/w), 0.055% (w/w), 0.06% (w/w), 0.0644% (w/w), 0.065% (w/w), 0.07% (w/w), 0.075% (w/w), 0.08% (w/w), 0.085% (w/w), 0.09% (w/w), 0.095% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w) or 1.0% (w/w) or any range or value therebetween. By way of even further example, but not limitation, the steroid can be present in the composition at about 0.0322% (w/w) based on the total weight of the composition. It should be understood that the amount of the steroid (or the agent with antimicrobial activity, if present) can refer to an active amount of the compound. By way of example, but not limitation, where 0.0644% (w/w) of betamethasone dipropionate is added, the active amount can be 0.05% (w/w) of betamethasone. Thus, in the examples, where an 0.05% betamethasone dipropionate cream is indicated, where the steroid is betamethasone dipropionate, the amount added to achieve 0.05% active betamethasone si 0.0644% (w/w).

Further exemplary, non-limiting dosage ranges of specific steroids for use in the cream compositions of the present methods are shown below in Table 1.

TABLE 1
Exemplary, non-limiting dosage ranges for corticosteroids (in mg/g of composition)

| | | | | | Biological |
|---|---|---|---|---|---|
| | | Preferred | More Preferred | Most Preferred | Half Life |
| Steroid | Dosage Range | Dosage Range | Dosage Range | Dosage Range | (Hours) |
| cortisone | 10.4-104.2 | 20.85-83.3 | 22.9-72.9 | 26-62.5 | 8-12 |
| hydrocortisone | 8.35-83.3 | 16.67-66.7 | 18.35-58.3 | 20.8-50 | 8-12 |
| Methyl- | 1.67-16.7 | 3.35-13.3 | 3.65-11.7 | 4.15-10 | 18-36 |
| prednisolone | | | | | |
| prednisolone | 2.1-20.8 | 4.15-16.7 | 4.6-14.6 | 5.2-12.5 | 18-36 |
| triamcinolone | 1.67-16.7 | 3.33-13.3 | 3.65-11.7 | 4.15-10 | 18-36 |
| Betamethasone | 0.25-2.5 | 0.5-2 | 0.55-1.75 | 0.625-1.5 | 36-54 |
| (free steroid) | | | | | |
| Betamethasone | 0.322-3.215 | 0.643-2.572 | 0.75-2.0 | 0.8-1.929 | 36-54 |
| dipropionate | | | | | |
| dexamethasone | 0.3-3.1 | 0.65-2.5 | 0.7-2.2 | 0.75-1.9 | 36-54 |

In any of the foregoing embodiments, the composition can comprise a total of about 0.01 mg to about 3 g of the steroid. By way of example, but not limitation, the amounts in Table 1 can be multiplied by 0.17 g, 0.34 g, 0.7 g, 1 g, 1.4 g, 2 g, 2.1 g, 4 g, 4.2 g, 5 g, 6 g, 8 g, 10 g or 20 g. By way of further example, but not limitation, the composition can comprise a total of about 0.01 mg to about 3 g, about 0.1 mg to about 3 g, about 0.5 mg to about 3 g, about 1 mg to about 3 mg, about 1.5 to about 3 mg, 0.01 mg to about 1.5 g, about 0.01 mg to about 1 g, about 0.01 mg to about 500 mg, about 0.01 mg to about 250 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 1.5 g, about 0.02 mg to about 1 g, about 0.02 mg to about 500 mg, about 0.02 mg to about 250 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 1.5 g, about 0.03 mg to about 1 g, about 0.03 mg to about 500 mg, about 0.03 mg to about 250 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 1.5 g, about 1 mg to about 1 g, about 1 mg to about 500 mg, about 1 mg to about 250 mg, about 1 mg to about 100 mg, about 1 mg to about 10 mg, about 1 mg to about 5 mg, about 2 mg to about 1.5 g, about 2 mg to about 1 g, about 2 mg to about 500 mg, about 2 mg to about 250 mg, about 2 mg to about 100 mg, about 2 mg to about 10 mg, about 2 mg to about 5 mg, about 8 mg to about 1.5 g, about 8 mg to about 1 g, about 8 mg to about 500 mg, about 8 mg to about 250 mg, about 8 mg to about 100 mg, about 8 mg to about 10 mg, about 10 mg to about 1.5 g, about 10 mg to about 1 g, about 10 mg to about 500 mg, about 10 mg to about 250 mg, about 10 mg to about 100 mg, about 100 mg to about 1.5 g, about 100 mg to about 1 g, about 100 mg to about 500 mg, about 100 mg to about 250 mg, about 250 mg to about 1.5 g, about 250 mg to about 1 g, about 250 mg to about 500 mg, about 500 mg to about 1.5 g, about 500 mg to about 1 g, about 1 g to about 1.5 g, about 0.01 mg, 0.02 mg, 0.05 mg, 0.1 mg, 0.125 mg, 0.15 mg, 0.2 mg, 0.25 mg, 0.3 mg, 0.4 mg, 0.5 mg, 0.6, mg, 0.7 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 220 mg, 240 mg, 250 mg, 260 mg, 280 mg, 300 mg, 330 mg, 350 mg, 360 mg, 390 mg, 400 mg, 440 mg, 450 mg, 480 mg, 500 mg, 550 mg, 600 mg, 650 mg, 700 mg, 750 mg, 800 mg, 850 mg, 900 mg, 950 mg, 1 g, 1.1 g, 1.2 g, 1.3 g, 1.4 g, or 1.5 g, 1.6 g, 1.7 g, 1.8 g, 1.9 g, 2 g, 2.1 g, 2.2 g, 2.3 g, 2.4 g, 2.5 g, 2.6, g, 2.7 g, 2.8 g, 2.9 g, or 3 g or any range or value therebetween.

In some embodiments, the steroid is betamethasone or betamethasone dipropionate and is present in a composition of the present disclosure at from about 0.322 mg to about 3.215 mg per gram of cream composition or from about 0.322 to about 0.644 mg per gram of cream composition, respectively, or from about 0.322 mg per gram of cream composition to about 0.644 mg per gram of cream composition. In other embodiments, the total dose of betamethasone dipropionate administered in a single application (i.e., bilateral intranasal administrations) is from about 0.322 mg to about 3.215 mg, or more preferably from about 0.80 mg to about 2.6 mg, or even more preferably from about 0.95 mg to about 1.93 mg, and even more preferably from about 1.28 mg to about 1.61 mg.

In any of the foregoing embodiments, the composition can further include an agent with antimicrobial activity. In any of the foregoing embodiments, the agent with antimicrobial activity can be present in the composition in an “effective amount.” The amount of agent with antimicrobial activity in compositions of the present disclosure can vary according to the desired dose to be delivered based on patient status, patient sensitivity, the route of administration, the biological half-life of the steroid, the patient's age, systemic factors and other factors. In addition, the state of the infection or disease and its susceptibility to the steroid can also be considered. One of skill in the art can determine an appropriate dosage, including determining an “effective amount” of the composition to apply.

Various antifungal agents can be used in the compositions and methods of the present disclosure. By way of example, but not limitation, such antifungal agents can include natamycin, ciclopirox, fluconazole, terbinafine, clotrimazole, itraconazole, ketoconazole, econazole, miconazole, nystatin, oxiconazole, terconazole, tolnaftate, efinaconazole, abafungin, terbinafine, butenafine, metronidazole and the like as well as combinations thereof and those known to one of skill in the art. In some embodiments, the antifungal agent is clotrimazole.

The antifungal agent can be present in the compositions of the present disclosure at an effective amount. In certain embodiments, the effective amount or total amount of antifungal agent per single administration (i.e., bilateral intranasal administration) is from about 20 mg to about 50 mg and more preferably from about 25 mg to about 40 mg. In certain embodiments, the antifungal agent is in an amount of from about 2.5 mg per gram of cream composition to about 10 mg per gram of cream composition, and more preferably, about 5 mg per gram cream composition. In some embodiments, the antifungal agent is present at about 0.1 to about 5 weight percent of the composition. By way of example but not limitation, the antifungal agent can be present at 0.1 to 5 weight percent of the composition, 0.5 to 4 weight percent of the composition, 0.5 to 3 weight percent of the composition, 0.5 to 2 weight percent of the composition, 0.5 to 1 weight percent of the composition, 1 to 5 weight percent of the composition, 2 to 5 weight percent of the composition, 3 to 5 weight percent of the composition, 4 to 5 weight percent of the composition or about 0.1, 0.2, 0.3, 0.4, 0.5, 0.6, 0.7, 0.8, 0.9, 1.0, 1.5, 2.0, 2.5, 3.0, 3.5, 4.0, 4.5 or 5.0 weight percent of the compositions. In some embodiments, the antifungal agent is clotrimazole and is present at about 0.5 weight percent of the composition.

In some embodiments, a composition of the present disclosure can further comprise, as an agent with antimicrobial activity, an antibiotic. By way of example, but not limitation, such antibacterial agents can include flucloxacilline, triclosan (2,4,4′-Trichloro-2′-hydroxydiphenyl ether), alcohols (including ethanol and isopropyl alcohol), peroxides (including benzoyl peroxide), iodine, benzethonium chloride, chloroxylenol and aminoglycoside antibiotics such as ciprofloxacin, and salts or derivatives thereof. By way of example but not limitation, other antibiotics can include amikacin, gentamicin, kanamycin, neomycin, netilmicin, streptomycin, tobramycin, paromycin, geldanamycin, herbimycin, loracarbef, ertapenem, doripenem, imipenem, meropenem, cefaclor, cefamandole, cefotoxin, cefprozil, cefuroxime, cefixime, cefdinir, cefditoren, cefpodoxime, ceftazidime, ceftibuten, ceftizoxime, ceftriaxone, cefepime, ceftobiprole, vancomycin, azithromycin, clarithromycin, dirithromycin, erythromycin, roxithromycin, troleandomycin, telithromycin, spectinomycin, aztreonam, amoxicillin, ampicillin, azociling, carbenicillin, cloxacillin, dicloxacillin, flucloxacillin, mezlocillin, meticillin, nafcillin, oxacillin, peperacillin, ticarcillin, bacitracin, colistin, polymyxin B, ciprofloxacin, clavulanic acid, enoxacin, gatifloxacin, levofloxacin, lomefloxacin, moxifloxacin, nonfloxacin, ofloxacin, trovafloxacin, grepafloxacin, sparfloxacin, AL-15469A, AL-38905, OP-145, mafenide, prontosil, sulfacetamide, sulfamethizole, sulfanilimide, sulfasalazine, sulfisoxazole, trimethoprim, cotrimoxazole, demeclocycline, doxycycline, minocycline, oxytetracycline, tetracycline, linezolid, arsogebanubem chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin, dalfopristin, rifampicin, thiamphenicol, tinidazole, amoxicillin/clavulanic acid, Maximin H5, Dermcidin, Cecropins, andropin, moricin, ceratotoxin, melittin, Magainin, dermaseptin, bombinin, brevinin-1, esculentins and buforin II, CAP18, LL37, abaecin, apidaecins, prophenin, indolicidin, brevinins, protegrin, tachyplesins, defensins, drosomycin, alamethicin, pexiganan or MSI-78, MSI-843, MSI-594, polyphemusin, colicin, pyocin, klebicin, subtilin, epidermin, herbicolacin, brevicin, halocin, agrocin, alveicin, carnocin, curvaticin, divercin, enterocin, enterolysin, erwiniocin, glycinecin, lactococin, lacticin, leucoccin, mesentericin, pediocin, plantaricin, sakacin, sulfolobicin, vibriocin, warnerinand, nisin, and the like, as well as salts or derivatives thereof.

In some embodiments, the agent with antimicrobial activity can be EDTA, such as, by way of example, but not limitation disodium EDTA.

In any of the foregoing embodiments, the agent with antimicrobial activity can be present in the composition in an amount of about 0.25% (w/w) to about 2% (w/w) based on the total weight of the composition. By way of example, but not limitation, the amount of the agent with antimicrobial activity in the composition can be about 0.25% (w/w) to about 1% (w/w), 0.5% (w/w) to about 1% (w/w), 0.5% (w/w) to about 2% (w/w), 1% (w/w) to about 2% (w/w), or 1.5% (w/w) to about 2% (w/w) based on the total weight of the composition. By way of further example but not limitation, the amount of the agent with antimicrobial activity in the composition can be about 0.25% (w/w), 0.3% (w/w), 0.4% (w/w), 0.5% (w/w), 0.6% (w/w), 0.7% (w/w), 0.75% (w/w), 0.8% (w/w), 0.9% (w/w), 1% (w/w), 1.25% (w/w), 1.5% (w/w), 1.75% (w/w) or 2% (w/w) or any range or value therebetween based on the total weight of the composition.

In any of the foregoing embodiments, the composition can comprise a total of about 0.01 mg to about 500 mg of the agent with antimicrobial activity. By way of example, but not limitation, the composition can comprise a total of about 0.01 mg to about 500 mg, about 0.1 mg to about 500 mg, about 1 mg to about 500 mg, about 5 mg to about 500 mg, about 10 mg to about 500 mg, about 100 mg to about 500 mg, about 200 mg to about 500 mg, about 300 mg to about 500 mg, about 400 mg to about 500 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 100 mg, about 1 mg to about 10 mg, about 1 mg to about 5 mg, about 2 mg to about 100 mg, about 2 mg to about 10 mg, about 2 mg to about 5 mg, about 8 mg to about 100 mg, about 8 mg to about 10 mg, about 10 mg to about 100 mg, about 50 mg to about 200 mg, about 50 mg to about 100 mg, about 100 mg to about 200 mg, about 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, or 500 mg or any range or value therebetween.

In any of the foregoing embodiments, the cream compositions of the present disclosure can include any suitable therapeutic active agent. The therapeutic active agents (including, but not limited to, steroids and/or antimicrobial agents) contemplated within the scope of the invention should be understood to include hydrophobic, hydrophilic and amphiphilic compounds. They may be in their free acid, free base, or pharmaceutically acceptable salt forms and include derivatives, esters or prodrugs. It should be understood that the cream compositions of the present disclosure can comprise only a steroid, only an antimicrobial agent (antifungal, antibacterial, or a combination thereof), or a combination of a steroid and an antimicrobial agent. The types of therapeutically active ingredients in the cream composition may be determined based on the condition treated and in some instances, may only require a steroid and in others only an antimicrobial agent, in further instances both a steroid and an antimicrobial agent, or in other instances a different therapeutic active agent. Thus, in some embodiments, the cream composition does not include an antimicrobial agent. In other embodiments, the cream composition does not include a steroid. In such instances, by way of example, but not limitation, the cream composition can include only a steroid as a therapeutic active agent, i.e. the cream composition does not include an antimicrobial agent. In other instances, by way of example, but not limitation, the cream composition can include only an antimicrobial agent as a therapeutic agent, i.e. the cream composition does not include a steroid. In some embodiments, the cream composition does not include either a steroid or an agent with antimicrobial activity.

In any of the foregoing embodiments, the composition can further include a stabilizing agent. In any of the foregoing embodiments, the stabilizing agent can be present in an amount sufficient to reduce the amount of degradants from the active pharmaceutical ingredients relative to a composition without the stabilizing agent after autoclaving, especially when autoclaving. By way of example, but not limitation, such autoclaving conditions can include 110° C. for 10 minutes, 110° C. for 30 minutes, 130° C. for 1 minute, 130° C. for 3 minutes, or 130° C. for 5 minutes. In any of the foregoing embodiments, the stabilizing agent can be edetic acid, pharmaceutically acceptable salts of edetic acid, citric acid, sodium citrate, fumaric acid, malic acid, maltose, pentetic acid, or a combination thereof and those known to one of skill in the art. Pharmaceutically acceptable salts of edetic acid can include any suitable salt, for example, disodium edetate. In any of the foregoing embodiments, the stabilizing agent can be present in the composition in an amount from about 0.005% (w/w) to about 0.25% (w/w) based on the total weight of the composition or any amount sufficient to reduce degradation of the pharmaceutically active compounds (or therapeutic active agent) in the composition relative to a composition without the stabilizing agent upon autoclaving. By way of example, but not limitation, the stabilizing agent can be present in the composition at about 0.005% (w/w), 0.01% (w/w), 0.015% (w/w), 0.025% (w/w), 0.05% (w/w), 0.075% (w/w), 0.1% (w/w), or 0.25% (w/w) or any range or value therebetween based on the total weight of the composition.

In any of the foregoing embodiments, the composition can be sterile. Sterility can be determined by, by way of example but not limitation, USP 71 testing.

Compositions of the present disclosure can be sterilized by any suitable means, preferably by autoclaving. By way of example, but not limitation, the composition can be sterilized by gamma irradiation or, in certain instances, by filtering. By way of further example, but not limitation, autoclaving at between 6-12 times the D-value of the composition can be sufficient to render the composition sterile, such as by measuring the survivor curve for Bacillus subtilis 5230 by standard methods for a given autoclave temperature.

In any of the foregoing embodiments, the composition can have a viscosity as measured by a Brookfield RVDVII+ with Spindle 28 at room temperature of (1) from about 200,000 centipoise (cPs) to about 2,000,000 cPs at a shear rate of about 0.3 RPM; (2) from about 100,000 cPs to about 1,500,000 cPs at a shear rate of about 0.5 RPM; (3) from about 100,000 cPs to about 1,000,000 at a shear rate of about 0.6 RPM; (4) from about 50,000 cPs to 800,000 cPs at a shear rate of about 0.8 RPM; (5) from about 50,000 cPs to about 750,000 cPs at a shear rate of about 1 RPM; (6) from about 40,000 cPs to about 500,000 cPs at a shear rate of about 1.5 RPM; (7) from about 30,000 cPs to about 250,000 cPs at a shear rate of about 2.0 RPM; (8) from about 20,000 cPs to about 200,000 cPs at a shear rate of about 2.5 RPM; (9) from about 20,000 cPs to about 200,000 cPs at a shear rate of about 3.0 RPM; (10) from about 15,000 cPs to about 150,000 cPs at a shear rate of about 4.0 RPM; (11) from about 15,000 cPs to about 150,000 cPs at a shear rate of about 5.0 RPM; (12) from about 10,000 cPs to about 100,000 cPs at a shear rate of about 6.0 RPM; (13) about 8,000 cPs to about 70,000 cPs at a shear rate of about 10.0 RPM; (14) from about 10,000 cPs to about 60,000 cPs at a shear rate of about 12.0 RPM; (15) from about 1,000 cPs to about 40,000 cPs at a shear rate of about 20.0 RPM; (16) from about 1,000 cPs to about 20,000 cPs at a shear rate of about 30.0 RPM; (17) from about 500 cPs to about 15,000 cPs at a shear rate of about 50.0 RPM; (18) from about 500 cPs to about 10,000 cPs at a shear rate of about 60.0 RPM; or (19) from about 250 cPs to about 7,000 cPs at a shear rate of about 100.0 RPM. In some embodiments, where the composition is sterile, the composition can have a viscosity as measured by a Brookfield RVDVII+ with Spindle 28 at room temperature of (1) from about 200,000 centipoise (cPs) to about 2,000,000 cPs at a shear rate of about 0.3 RPM; (2) from about 100,000 cPs to about 1,500,000 cPs at a shear rate of about 0.5 RPM; (3) from about 100,000 cPs to about 1,000,000 at a shear rate of about 0.6 RPM; (4) from about 100,000 cPs to 800,000 cPs at a shear rate of about 0.8 RPM; (5) from about 100,000 cPs to about 750,000 cPs at a shear rate of about 1 RPM; (6) from about 50,000 cPs to about 500,000 cPs at a shear rate of about 1.5 RPM; (7) from about 50,000 cPs to about 250,000 cPs at a shear rate of about 2.0 RPM; (8) from about 30,000 cPs to about 200,000 cPs at a shear rate of about 2.5 RPM; (9) from about 30,000 cPs to about 200,000 cPs at a shear rate of about 3.0 RPM; (10) from about 20,000 cPs to about 150,000 cPs at a shear rate of about 4.0 RPM; (11) from about 20,000 cPs to about 150,000 cPs at a shear rate of about 5.0 RPM; (12) from about 15,000 cPs to about 100,000 cPs at a shear rate of about 6.0 RPM; (13) about 10,000 cPs to about 70,000 cPs at a shear rate of about 10.0 RPM; (14) from about 10,000 cPs to about 60,000 cPs at a shear rate of about 12.0 RPM; (15) from about 1,000 cPs to about 40,000 cPs at a shear rate of about 20.0 RPM; (16) from about 1,000 cPs to about 20,000 cPs at a shear rate of about 30.0 RPM; (17) from about 500 cPs to about 15,000 cPs at a shear rate of about 50.0 RPM; (18) from about 500 cPs to about 10,000 cPs at a shear rate of about 60.0 RPM; or (19) from about 250 cPs to about 7,000 cPs at a shear rate of about 100.0 RPM. Alternatively, in any of the foregoing embodiments, the composition can have a viscosity measured by a Brookfield Rheometer DV3T CP Rheometer with spindle CP52 at 25.0+/−0.1° C. of: (1) from about 30,000 cPs to about 500,000 cPs at a shear rate of about 0.3 RPM; (2) from about 30,000 cPs to about 300,000 at a shear rate of about 0.6 RPM; (3) from about 10,000 cPs to about 200,000 cPs at a shear rate of about 1.5 RPM; (4) from about 7,000 cPs to about 70,000 cPs at a shear rate of about 3.0 RPM; (5) from about 3,000 cPs to about 20,000 cPs at a shear rate of about 12.0 RPM; (6) from about 300 cPs to about 7,000 cPs at a shear rate of about 30.0 RPM; or (7) from about 150 cPs to about 3,500 cPs at a shear rate of about 60.0 RPM. In some embodiments, where the composition is sterile, the composition can have a viscosity measured by a Brookfield Rheometer DV3T CP Rheometer with spindle CP52 at 25.0+/−0.1° C. of: (1) from about 70,000 cPs to about 700,000 cPs at a shear rate of about 0.3 RPM; (2) from about 30,000 cPs to about 300,000 at a shear rate of about 0.6 RPM; (3) from about 10,000 cPs to about 200,000 cPs at a shear rate of about 1.5 RPM and a torque of 10-100%; (4) from about 10,000 cPs to about 70,000 cPs at a shear rate of about 3.0 RPM; (5) from about 3,000 cPs to about 20,000 cPs at a shear rate of about 12.0 RPM; (6) from about 300 cPs to about 7,000 cPs at a shear rate of about 30.0 RPM; or (7) from about 150 cPs to about 3,500 cPs at a shear rate of about 60.0 RPM. It should be understood that, as used in the present disclosure, room temperature can be a temperature from 20° C. to 25° C.

In any of the foregoing embodiments, the composition can be a water-in-oil emulsion or an oil-in-water emulsion. In such embodiments, the composition can have a globule size or particle size of less than 50 μm, 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, or 1 μm, whether by either number mean or volume mean. By way of example, but not limitation, the globule size or particle size of the composition can be from about 1 μm to about 50 μm, 1 μm to about 45 μm, 1 μm to about 40 μm, 1 μm to about 35 μm, 1 μm to about 30 μm, 1 μm to about 25 μm, 1 μm to about 20 μm, 1 μm to about 15 μm, 1 μm to about 10 μm, about 1.5 μm to about 50 μm, about 1.5 μm to about 45 μm, about 1.5 μm to about 40 μm, about 1.5 μm to about 35 μm, about 1.5 μm to about 30 μm, about 1.5 μm to about 25 μm, about 1.5 μm to about 20 μm, about 1.5 μm to about 15 μm, about 1.5 μm to about 10 μm, about 2.0 μm to about 50 μm, about 2.0 μm to about 45 μm, about 2.0 μm to about 40 μm, about 2.0 μm to about 35 μm, about 2.0 μm to about 30 μm, about 2.0 μm to about 25 μm, about 2.0 to about 20 μm, about 2.0 μm to about 15 μm, about 2.0 μm to about 10 μm, about 3.0 μm to about 50 μm, about 3.0 μm to about 45 μm, about 3.0 μm to about 40 μm, about 3.0 μm to about 35 μm, about 3.0 μm to about 30 μm, about 3.0 μm to about 25 μm, about 3.0 μm to about 20 μm, about 3.0 μm to about 15 μm, about 3.0 to about 10 μm, about 5.0 μm to about 50 μm, about 5.0 μm to about 45 μm, about 5.0 μm to about 40 μm, about 5.0 μm to about 35 μm, about 5.0 μm to about 30 μm, about 5.0 μm to about 25 μm, about 5.0 μm to about 20 μm, about 5.0 μm to about 15 μm, about 5.0 μm to about 10 μm, about 1 μm to about 5 μm, about 1.5 μm to about 5 μm, about 2 μm to about 5 μm, about 3 μm to about 5 μm, about 5 μm to about 10 μm, about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.25, 3.5, 3.75, 4, 4.25, 4.5, 4.75, 5, 5.5, 6, 6.5, 7, 7.5, 8, 8.5, 9, 9.5, 10, 15, 20, 25, 30, 35, 40, 45 or 50 μm or any range or value therebetween either by number mean or volume mean. In some embodiments, the composition is sterile and has a measurable globule size. It should be understood that for an oil-in-water emulsion, the globule size refers to oil globule size. It should be further understood that globule size or particle size can be as measured by USP 729.

In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% Relative Humidity (RH) for 1 month. In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 3 months. In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 6 months. In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% Relative Humidity (RH) for 12 months. In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% Relative Humidity (RH) for 18 months. In any of the foregoing embodiments, the composition can have a globule size or particle size, as measured by number mean, that does not change by more than 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% Relative Humidity (RH) for 24 months. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% Relative Humidity (RH) for 1 month. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 3 months. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 6 months. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 12 months. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 18 months. In any of the foregoing embodiments, the composition can have a globule size or particle size, as measured by volume mean, that does not change by more than 100%, 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% when stored at 25° C./60% RH for 24 months. By way of example, but not limitation, globule size or particle size can be as measured by USP 729. It should be understood that for an oil-in-water emulsion, the globule size refers to oil globule size.

In any of the foregoing embodiments, the composition does not agglomerate, cream, sediment, flocculate, phase invert, or coalesce after storage at 25° C./60% Relative Humidity for 1 month, 3 months, 6 months, 12 months, 18 months, or 24 months. In any of the foregoing embodiments, the composition does not agglomerate, cream, sediment, flocculate, phase invert, or coalesce after storage at 40° C./70% Relative Humidity for 1 month, 3 months, 12 months, 18 months, or 24 months. Agglomeration can be understood as the combination of globules to form larger globules. Creaming and sedimentation can be understood as the combination of globules to form larger globules which are no longer dispersed either at the top or the bottom of the composition, respectively. Flocculation can be understood as aggregation of droplets without an increase in primary droplet size into larger units. Phase inversion can be understood as where there is an exchange between the dispersed phase and the medium, such as an o/w emulsion becoming a w/o emulsion or vice versa. Coalescence can be understood as the fusion of droplets to form larger droplets due to thinning and disruption of the liquid film between droplets which can result in phase separation.

In any of the foregoing embodiments, the composition can not permeate cadaver skin or nasal mucosa after 0.5, 1, 2, 4, 6, 8, 12, 24 or 48 hours. In any of the foregoing embodiments, the composition can not permeate cadaver skin or nasal mucosa at >45 ng/mL after 0.5, 1, 2, 4, 6, 8, 12, 24 or 48 hours.

In any of the foregoing embodiments, the composition can include less than 10% total degradants from the steroid or agent with antimicrobial activity, if present. By way of example, but not limitation, the composition can comprise less than 10%, 9%, 8%, 7%, 6%, 5%, 4%, 3%, 2%, 1%, 0.5% or 0.1% total degradants from the steroid or agent with antimicrobial activity. By way of further example, but not limitation, the total degradants can be as measured by HPLC. By way of still further example, but not limitation, the total degradants can include betamethasone (EP Impurity A, CAS No. 378-44-9), betamethasone 17-propionate (EP Impurity B, CAS No. 5534-13-4), betamethasone 21-propionate (EP Impurity C, CAS No. 75883-70-7), betamethasone 21-acetate 17-propionate (EP Impurity D, CAS No. 5514-81-8), beclomethasone dipropionate (EP Impurity E, CAS No. 5534-09-8), betamethasone 9,11-epoxide 17,21-dipropionate (EP Impurity F, CAS No. 66917-44-0), beclometasone tripionate (EP Impurity G, CAS No. 1186048-33-8), 6-bromo-betamethasone-17,21,dipropionate (EP Impurity H, CAS No. 1186048-34-9), (8S,9R,10S,11S,13S,14S,16S,17R)-9-fluoro-1-hydroxy-10,13,16-trimethyl-3-oxo-17-(2-(propionyloxy)acetyl)-2,3,6,7,8,9,10,11,12,13,14,15,16,17-tetradecahydro-1H-cyclopenta[a]phenanthren-17-yl propionate (EP Impurity I, CAS No. 80163-83-3), and combinations thereof. In certain aspects the total degradants can include betamethasone EP Impurities A, B, C, D, E, F, G, H and I.

In any of the foregoing embodiments, the composition can include less than 10% total degradants from the steroid or agent with antimicrobial activity, if present, after storage at 25° C./60% RH for 1 month or 3 months. By way of example, but not limitation, the compositions can include less than 10%, 9%, 8%, 7%, 6%, 5%, 4%, 3%, 2%, 1%, 0.5%, or 0.1% total degradants from the steroid or agent with antimicrobial activity after storage at 25° C./60% RH for 1 month or 3 months. By way of further example, but not limitation, the total degradants can be as measured by HPLC.

In any of the foregoing embodiments, the composition can have a steroid or agent with antimicrobial activity content, if present, that is within 10% of a starting content, as measured after storage at 25° C./60% RH for 1 month or 3 months. By way of example, but not limitation, the composition can have a steroid or agent with antimicrobial activity content, if present, that is within 10%, 9%, 8%, 7%, 6%, 5%, 4%, 3%, 2%, or 1% of the starting content, as measured after storage at 25° C./60% RH for 1 month or 3 months. By way of further example, but not limitation, the steroid or agent with antimicrobial activity content can be as measured by liquid chromatography (LC), such as HPLC. By way of example, but not limitation, the steroid can be betamethasone dipropionate or betamethasone.

In any of the foregoing embodiments, the composition can have a pH of about 3.5 and about 8, preferably about 4 and about 7, more preferably about 5 and about 6. By way of example, but not limitation, the pH-modifying agent can be present in amount sufficient to adjust the pH of the composition to about 3.5 to about 8, about 4 to about 7, about 5 to about 7, about 5 to about 6, about 6 to about 7, about 4 to about 6, about 4 to about 5, or about 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 and any range or value therebetween.

In any of the foregoing embodiments, the composition can have a pH that is within 0.5 of a starting pH of the composition, as measured after storage at 25° C./60% RH for 1 month or 3 months. By way of example, but not limitation, the composition can have a pH that is within 0.5, 0.4, 0.3, 0.2 or 0.1 of the starting pH, as measured after storage at 25° C./60% RH for 1 month or 3 months. By way of further example, but not limitation, pH can be measured by standard methods.

In any of the foregoing embodiments, the composition can have an osmolality that is within 10 mOsm/kg of a starting osmolality of the composition, as measured after storage at 25° C./60% RH for 1 month or 3 months. By way of example, but not limitation, the composition can have an osmolality that is within 10, 9, 8, 7, 6, 5, 4, 3, 2, or 1 mOsm/kg of the starting osmolality, as measured after storage at 25° C./60% RH for 1 month or 3 months. By way of further example, but not limitation, the osmolality can be as measured by USP 785.

In any of the foregoing embodiments, the composition can have a viscosity that is within 10% of a starting viscosity of the composition, as measured after storage at 25° C./60% for 1 month or 3 months. By way of example, but not limitation, the composition can have a viscosity that is within 10%, 9%, 8%, 7%, 6%, 5%, 4%, 3%, 2% or 1% of the starting viscosity, as measured after storage at 25° C./60% RH for 1 month or 3 months. By way of example, but not limitation, the viscosity can be as measured by the methods for viscosity measurement described in the present disclosure.

It should be understood that for any of the starting properties, e.g. starting viscosity, that these properties can be as measured at the time storage is commenced or from the time of formulation which may coincide.

In any of the foregoing embodiments, the composition can not include a pro-inflammatory cytokine inhibitor.

In any of the foregoing embodiments, the only pharmaceutically active compounds in the composition can be the steroid or the agent with antimicrobial activity. In any of the foregoing embodiments, the composition can not contain any pharmaceutically active compound that is not the steroid or the agent with antimicrobial activity.

In any of the foregoing embodiments, the composition can be packaged in a syringe or other vessel.

Various topical analgesics can also be included in the compositions described herein. These include, but are not limited to, nonsteroidal anti-inflammatory drugs, lidocaine, capsaicin, amitriptyline, glyceryl trinitrate, opioids, menthol, pimecrolimus, phenytoin and the like.

In any of the foregoing embodiments, the composition can be an cream, in certain aspects an isotonic cream, that can withstand autoclaving without separating into its component phases. By way of example, but not limitation, such autoclaving conditions can include 110° C. for 10 minutes, 110° C. for 19 minutes, 110° C. for 30 minutes, 130° C. for 1 minute, 130° C. for 3 minutes, or 130° C. for 5 minutes. In some embodiments, the lack of separation can be assessed by measuring globule size. In some embodiments, the composition does not agglomerate, cream, sediment, flocculate, phase invert, coalesce, or a combination thereof after autoclaving, such as, by way of example, but not limitation, under the conditions recited. To the extent that a composition retains a globule size, it has not separated and is still an emulsion. By way of example, but not limitation, the lack of separation can also be assessed by measuring the change in globule size before and after sterilization. By way of example, the globule size, either by number mean or volume mean, can be within about 1%, 2%, 3%, 4%, 5%, 10%, 15%, 20%, 25%, 30%, 35%, 40%, 50%, 60%, 75%, 100% or 200% after sterilization relative to before sterilization. Such isotonic creams can be sterile and can be used as compositions for delivery of therapeutic agents to tissues, including tissues of the nose and ear, as well as any mucosal tissue such as, by way of example, but not limitation, the ophthalmic, vaginal, rectal or urethral as well as nasal, sinonasal, nasopharyngeal and otic tissues or any other mucosal tissue.

It should be understood that in any of the foregoing embodiments, the tonicity modifiers and emulsifiers can be used as tonicity agents to the extent that they alter tonicity, and that the amounts and types of tonicity agents, tonicity modifiers and emulsifiers used can be sufficient to produce a composition with the desired tonicity. Thus, it should be understood that the tonicity modifiers and emulsifiers can be substituted for tonicity agents. For example, in certain compositions, a tonicity agent is not required if the tonicity modifiers and/or emulsifiers are sufficient to yield the desired tonicity.

It should be understood that one of skill in art can design and/or produce the compositions of the present disclosure to have the desired tonicity based on any suitable method. By way of example, but not limitation, the sodium chloride equivalency method can be used to calculate the expected osmolality of a composition based on its ingredients.

It should be understood that the features and aspects of the compositions of the present disclosure can be combined in various combinations by one of skill in the art without deviating from the scope of the present disclosure. By way of example, but not limitation, an autoclavable cream composition or a sterile cream composition can include the properties in the foregoing embodiments and can have the osmolality recited or a different osmolality. By way of example, but not limitation, the osmolality can be isotonic to the mucosal tissue to which the composition is to be applied.

An exemplary composition of the present disclosure is provided in Table 2 below.

TABLE 2
Exemplary Cream Composition of the Present Disclosure

| Ingredient | % w/w | Function/Genus | Critical Role |
|---|---|---|---|
| Clotrimazole | 1.00 | Active antifungal with | Antimicrobial |
| | | antibacterial properties | |
| Betamethasone | 0.0322 | Active steroid | Anti-inflammatory |
| dipropionate | | | |
| Polysorbate 80 | 5.00 | Emulsifying Agent; | Emulsifier, Tonicity, |
| | | Nonionic Surfactant; | Solubilizer, |
| | | Solubilizing Agent; | Emulsion Stabilizer |
| | | Tonicity Agent | |
| Glycerin | 1.75 | Humectant; Solvent; | Tonicity, Solvent |
| | | Tonicity Agent | |
| EDTA Disodium | 0.05 | Chelating Agent; | Chemical Stability, |
| “Disodium Edetate” | | Sequestrant Antimicrobial | Chelating Agent, |
| | | | Antimicrobial |
| Carbopol 980 | 0.60 | Bioadhesive; Emulsifying | Viscosity, Emulsion |
| | | Agent; Emulsion Stabilizer; | Stabilizer, pH, |
| | | Rheology Modifier | Bioadhesion |
| Polyoxyl 40 Stearate | 1.00 | Emulsifying Agent; | Emulsifier |
| | | Solubilizing Agent; | |
| | | Wetting Agent | |
| Cetyl Alcohol | 1.00 | Emulsifying Agent; | Emulsifier |
| | | Stiffening Agent | |
| Glyceryl Monostearate | 0.50 | Emollient; Emulsifying | Emulsifier |
| | | Agent; Solubilizing Agent | |
| Petrolatum | 8.00 | Emollient | Emollient |
| Oleth-2 or Span 20 | 3.00 | Emulsifying Agent; | Emulsifier |
| | | Penetration Enhancer; | |
| | | Solubilizing Agent; | |
| | | Wetting Agent | |
| Benzyl Alcohol | 0.90 | Preservative; | Preservative, |
| | | Tonicity Agent | Tonicity |
| 1% NaOH | Adjust | Alkalizing Agent | pH |
| | pH | | |
| | to 5-6 | | |
| Purified Water | QS | Vehicle; Solvent | Vehicle |

Exemplary ranges for each of the components in Table 2 above are provided in Table 3 below. It should be understood that the exemplary cream compositions of Tables 2 and 3 can also be prepared without clotrimazole.

TABLE 3
Exemplary Ranges for Components in Exemplary
Cream Composition of Table 2

| Ingredient | % w/w | Range |
|---|---|---|
| Clotrimazole | 1.00 | 0.25-2% |
| Betamethasone | 0.0322 | 0.015-0.322% |
| dipropionate | | |
| Polysorbate 80 | 5.00 | 0.1-15% |
| Glycerin | 1.75 | 0.1-15% |
| EDTA Disodium | 0.05 | 0.005-0.25% |
| “Disodium Edetate” | | |
| Carbopol 980 | 0.60 | 0.1-1% |
| Polyoxyl 40 Stearate | 1.00 | 0.25-10% |
| Cetyl Alcohol | 1.00 | 0.25-10% |
| Glyceryl Monostearate | 0.50 | 0.1-5% |
| Petrolatum | 8.00 | 4-30% |
| Oleth-2 or Span 20 | 3.00 | 0.5-10% |
| Benzyl Alcohol | 0.90 | 0.5-15% |
| 1% NaOH | Adjust | |
| | pH | |
| | to 5-7 | |
| Purified Water | QS | 1-99% |

### Methods of Manufacturing

The present disclosure also provides methods for manufacturing the compositions of the present disclosure. It should be understood that the methods described herein are not meant to exclude other methods for producing the compositions of the present disclosure.

In some embodiments, a method for manufacturing a cream includes the steps of preparing an aqueous phase dispersion, preparing an oil phase dispersion, and combining the aqueous phase dispersion and the oil phase dispersion to form an emulsion mixture. In some embodiments, the pH is adjusted during the step of forming the aqueous phase dispersion. In other embodiments, the pH is adjusted after combining the aqueous phase dispersion and the oil phase dispersion to form an emulsion mixture. By way of example, but not limitation, the pH of the aqueous dispersion or emulsion mixture can be adjusted to about 3.5 and about 8, preferably about 4 and about 7, more preferably about 5 and about 6. By way of example, but not limitation, the pH of the aqueous dispersion can be adjusted to about 3.5 to about 8, about 4 to about 7, about 5 to about 7, about 5 to about 6, about 6 to about 7, about 4 to about 6, about 4 to about 5, or about 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 and any range or value therebetween. In some embodiments the pH of the aqueous dispersion is adjusted by adding a pH-modifying agent as described herein.

In some embodiments, the aqueous phase dispersion is prepared by forming the aqueous phase dispersion, adjusting the pH of the dispersion, adding at least one emulsifier to the aqueous phase dispersion, heating the aqueous dispersion containing the at least one emulsifier, and adding a first portion of at least one pharmaceutically active compound to the aqueous phase dispersion. By way of example, but not limitation, heating can be to about 25-80° C. for a sufficient period of time to form the dispersion. In other embodiments, the aqueous phase dispersion is prepared by forming the aqueous phase dispersion and adding a first portion of at least one pharmaceutically active compound to the aqueous phase dispersion, where the pH is not adjusted prior to adding the first portion of at least one pharmaceutically active compound. In some embodiments, the step of forming the aqueous phase dispersion can further include adding a tonicity agent to the aqueous phase dispersion. In some embodiments, the step of forming the aqueous phase dispersion can further include adding a vehicle as described herein. In some embodiments, the step of forming the aqueous dispersion can further include adding a stabilizing agent as described herein. In some embodiments, the step of forming the aqueous dispersion can further include adding a viscosity modifying agent as described herein.

In parallel, to the preparation of the aqueous phase dispersion, an oil phase dispersion can be prepared. In some embodiments, the oil phase is prepared by heating the oil phase and adding a second portion of the at least one pharmaceutically active compound to the oil phase. By way of example, but not limitation, heating can be to about 25-80° C. for a sufficient period of time to form the dispersion. In some embodiments, the oil phase comprises an emulsifier as described herein which can be the same or different from an emulsifier in the aqueous phase dispersion, if one is added. In some embodiments, at least one emulsifier can be added during the preparation of the oil phase dispersion. In some embodiments, the preparation of the oil phase dispersion can further include adding an emollient to the oil phase dispersion.

After the aqueous phase dispersion and the oil phase are produced, the aqueous phase dispersion and the oil phase are combined to produce an emulsion mixture. Preferably, the oil phase is still hot, e.g. above 30° C., at the time of combining the two phases. After combining the two phases, the emulsion mixture can be cooled. In some embodiments, once the emulsion mixture is cooled, e.g. below 30° C., a tonicity modifier and/or a preservative, which can be the tonicity modifier, as described herein can be added to the emulsion mixture.

In some embodiments, if the pH was not adjusted in the aqueous phase dispersion, the pH of the emulsion mixture can be adjusted. In such embodiments, after the tonicity modifier and/or preservative is added, an emulsifier as described herein can be added to emulsion mixture and the emulsion mixture heated. By way of example, but not limitation, heating can be to about 25-80° C. for a sufficient period of time to form the dispersion.

After the emulsion mixture has been prepared, the composition can be filled into a vessel and subjected to sterilization such as, by way of example, but not limitation, by autoclaving. By way of example, but not limitation, sterilization can be performed by autoclaving at 110° C. for 10-30 minutes or 130° C. for 1-5 minutes. By way of example, but not limitation, the vessel can be a syringe. By way of further example, but not limitation, the sterilization can be by gamma irradiation, such as 15-25 mGy, or by filtration that does not separate the phases of the cream.

It should be understood that in the foregoing manufacturing embodiments, the amounts of the components can be added to arrive at a composition of the present disclosure and that certain components or steps can be omitted or rearranged based on the composition and the knowledge of one of ordinary skill in the art.

It should also be understood that the components of the aqueous phase dispersion and the oil phase dispersion and of the final emulsion mixture can be as described throughout the present disclosure. By way of example, but not limitation, the emulsifier, emollient, tonicity agent, tonicity modifier, viscosity modifier, stabilizer, vehicle and pH-modifying agent can be as described in the other portions of the present disclosure.

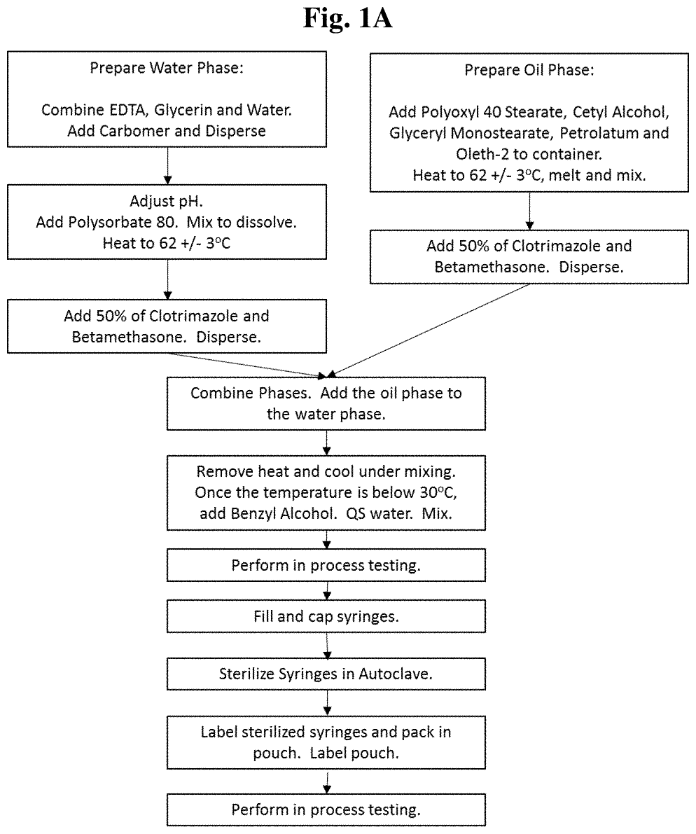

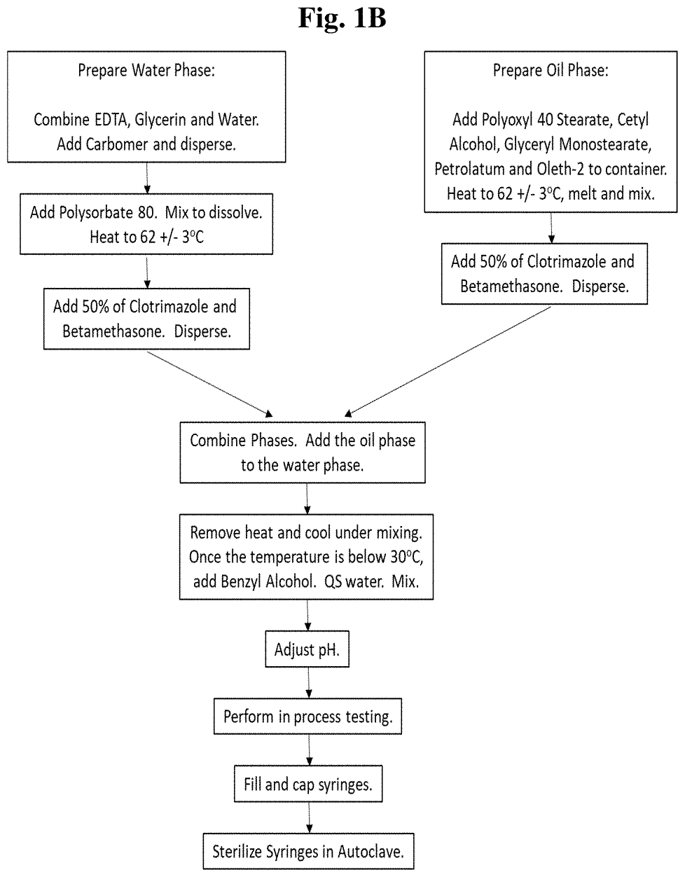

Exemplary methods for producing the compositions of the present disclosure are provided in FIGS. 1A and 1B. It should be understood that these methods can be modified to substitute Span 20 for Oleth-2.

It should be understood that in any of the foregoing embodiments for methods of manufacturing, the heating steps can be to heat the dispersion or mixture to about 25-80° C. By way of example, but not limitation, the heating can be to about 25 to about 80° C., about 30 to about 80° C., about 35 to about 80° C., about 40 to about 80° C., about 50 to about 80° C., about 60 to about 80° C., about 70 to about 80° C., about 30 to about 70° C., about 40 to about 70° C. about 50 to about 70° C., about 60 to about 70° C., about 30 to about 60° C., about 40 to about 60° C., about 50 to about 60° C., about 30 to about 50° C., about 40 to about 50° C., about 25, 30, 35, 40, 45, 50, 55, 60, 65, 70, 75, or 80° C. or any range or value therebetween.

### Devices

The present disclosure provides for devices for the application of compositions to the nasal and otic tissues. Such devices can be used to apply compositions, such as the cream compositions of the present disclosure, to any tissue of the nose or ear. By way of example, but not limitation, the devices can be used to deliver the composition to the sinus or nasopharyngeal tissues, such as frontal, ethmoid, maxillary, and sphenoid tissues. By way of further example, but not limitation, the devices can be used to deliver the composition to tissues of the ear such as the auricle, cochlea, ear canal, Eustachian tube, external auditory canal, inner ear, middle ear, outer ear, round window, semicircular canals, tympanic membrane, tympanic cavity, meatal tissue or hair cells.

In some embodiments, a device of the present disclosure can include a length of tubing having a first end and a second end disposed at opposite ends of the length of tubing, where the length of tubing has an outer diameter at the first end, a structural support element passing through the tubing from the first end toward the second end for at least a portion of the length of the length of tubing, a tip disposed at the second end which has an arcuate shape that tapers at the end away from the second end of the length of tubing and has a largest outer diameter at an end proximate to the second end of the length of tubing, the largest outer diameter being larger than the outer diameter at the first end of the length of tubing and the structural support element having sufficient rigidity to hold the shape of the tubing, but sufficient flexibility for the shape of the tubing to be altered. The arcuate shaped tip, which can be called a “mushroom” tip, can be useful to spread the cream compositions of the present disclosure onto mucosal tissue and to navigate tissues to deliver the cream composition to the appropriate tissue. In some embodiments, a device of the present disclosure can include a length of tubing having a first end and a second end disposed at opposite ends of the length of tubing, where the length of tubing has an outer diameter at the first end and a tip disposed at the second end which has an arcuate shape that tapers at the end away from the second end of the length of tubing and has a largest outer diameter at an end proximate to the second end of the length of tubing, the largest outer diameter being larger than the outer diameter at the first end of the length of tubing. In such latter embodiments, the tubing can be rigid and/or include a bend.

In some embodiments, the length of the length of tubing can be from about 0.5 to about 10 inches. By way of example, but not limitation, the length of the length of tubing can be about 0.5, 0.75, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 inches long or any range or value therebetween. In some embodiments, the structural support element can be a wire or other bendable structure. By way of example, but not limitation, the structural support element can be a plastic or metal wire. In some embodiments, the structural support element runs the entire length of the length of tubing. In some embodiments, the structural support element runs about half the length of the length of tubing. In some embodiments, the device does not include the structural support element and the length of tubing is rigid. In some embodiments, the device does not include the arcuate tip or structural support element and the length of tubing is rigid. In some embodiments, the rigid design can include a bend in the length of tubing. In some embodiments, the rigid design can include a second length of tubing attached to the first which has a smaller diameter than the length of the tubing. In such instances, the diameters can be as described herein.

In some embodiments, the device can further include a connector at the first end configured to be attached to a syringe. In some embodiments, the device further includes a syringe connected to the device at the first end by a connector. By way of example, but not limitation, the connector and be a leur lock connector. In some embodiments, the tubing is made of a flexible material such as, by way of example, but not limitation, a polyether block amide such as Pebax 45D, Pebax 55D or Pebax 63D. In some embodiments, the polyether block amide, or other tubing material, can be modified with an additive to reduce the coefficient of friction of the polyether block amide. By way of example, but not limitation, the dry static coefficient of friction against stainless steel of the tubing material as measured by ASTM D1894 testing can be less than 0.2. By way of example, the dry static coefficient of friction against stainless steel of the tubing material as measured by ASTM D1894 can be less than 0.2, 0.15, 0.1, 0.05, or 0.025 and any range of value therebetween. By way of further example, but not limitation, the dry static coefficient of friction against stainless steel of the tubing material as measured by ASTM D1894 can be between about 0.025 and about 0.2, about 0.025 and about 0.15, about 0.025, and about 0.1, about 0.025 and about 0.05 and any range of value therebetween. In some embodiments, the material can have a flexural modulus of between about 100 and about 400 MPa as measured by ASTM D790 testing. By way of example, but not limitation, the flexural modulus of the material can be between about 100 and about 400 MPa, about 150 and about 300 MPa, about 150 and about 200 MPa, about 200 and 300 MPa, about 100, 125, 150, 164, 175, 200, 225, 250, 275, 278 or 300 MPa and any range or value therebetween as measured by ASTM D790 testing. In some embodiments, the material can have a shore hardness (Shore D) as measured by ASTM D2240 testing of about 35 to about 80 under either instantaneous or after 15 second conditions. By way of example, but not limitation, the material can have a shore hardness (Shore D) as measured by ASTM D2240 of about 35 to about 80, about 40 to about 80, about 45 to about 75, about 50 to about 70, about 50 to about 60, about 40, 41, 45, 46, 50, 54, 55, 58, 60, 64, 65, 70, 75 or 80 and any range or value therebetween under either instantaneous or after 15 second conditions. In some embodiments, the tubing can be made from a rigid material such as, by way of example, but not limitation, high density polyethylene (HDPE). In some embodiments, the tip and the tubing can be made from the same material. In some embodiments, the largest outer diameter of the tip can be about 4 mm. By way of example, but not limitation, the largest outer diameter of the tip can be about 1.5 mm to about 6 mm, about 2 mm to about 6 mm, about 2 mm to about 4 mm, about 4 mm to about 6 mm, about 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6 mm or any value or range therebetween. In some embodiments, the tip has a length of about 1 mm. By way of example but not limitation, the tip can have a length of about 0.5 mm to about 10 mm, about 0.5 mm to about 2 mm, about 0.5 mm to about 1 mm, about 1 mm to about 2 mm, about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, or 10 mm and any range or value therebetween. In some embodiments, the outer diameter of the first end of the length of tubing can be about 2 mm. By way of example, but not limitation, the outer diameter of the first end can be about 1 mm to about 4 mm, about 1 mm to about 2 mm, about 1 mm to about 3 mm, about 2 mm to about 4 mm, about 3 to about 4 mm, about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 mm or any value or range therebetween. In some embodiments, the tubing has an inner diameter of about 1.4 mm. By way of example, but not limitation, the inner diameter of the tubing can be about 1 mm to about 2 mm, about 1 mm to about 1.5 mm, about 1.5 mm to about 2 mm, about 1, 1.1, 1.2, 1.3, 1.4, 1.5, 1.6, 1.7, 1.8, 1.9, or 2 mm or any value or range therebetween. In some embodiments, the structural support element has a diameter of about 0.5 mm. By way of example, but not limitation, the structural support element can have a diameter of about 0.1 mm to about 1 mm, about 0.5 mm to about 1 mm, about 0.1 mm to about 0.5 mm, about 0.1, 0.2, 0.3, 0.4, 0.5, 0.6, 0.7, 0.8, 0.9, or 1 mm or any value or range therebetween. In some embodiments, the device is sterile.

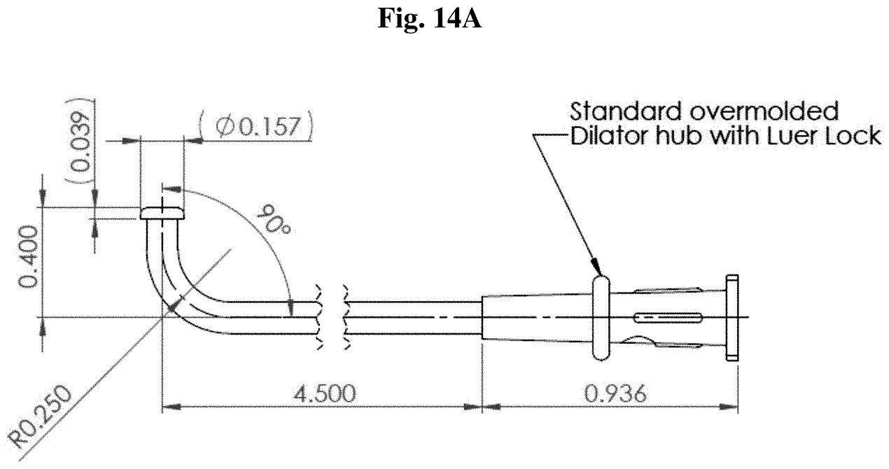

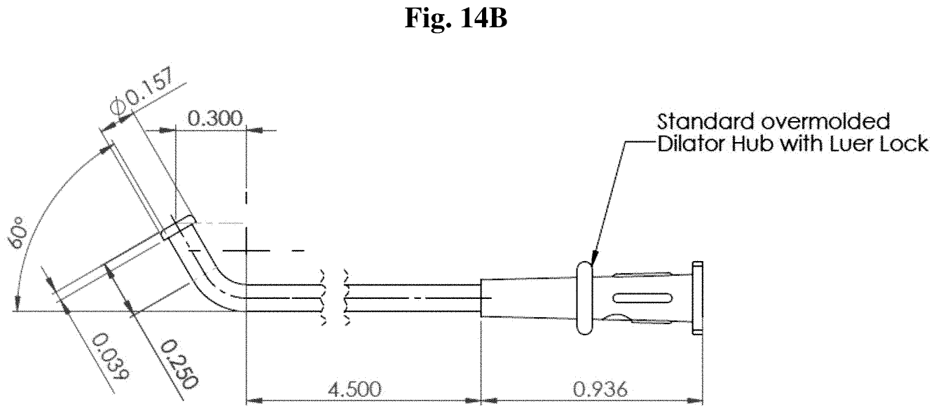

It should be understood that in the foregoing embodiments of the device, the length of tubing can have any suitable shape and that the diameter can refer to the length or width of the tubing if it is non-circular. By way of example, but not limitation, the device can have a bend between the first end and the second end, wherein the bend has a degree of curvature of about 60° relative to an axis passing from the first end to the bend. By way of further example, but not limitation, the bend can be from about 0° to about 90°, from about 0° to about 80°, from about 0° to about 70°, from about 0° to about 60°, from about 0° to about 50°, from about 0° to about 45°, from about 0° to about 40°, from about 0° to about 30°, from about 0° to about 20°, from about 0° to about 10°, from about 10° to about 90°, from about 10° to about 80°, from about 10° to about 70°, from about 10° to about 60°, from about 10° to about 50°, from about 10° to about 45°, from about 10° to about 40°, from about 10° to about 30°, from about 10° to about 20°, from about 20° to about 90°, from about 20° to about 80°, from about 20° to about 70°, from about 20° to about 60°, from about 20° to about 50°, from about 20° to about 45°, from about 20° to about 40°, from about 20° to about 30°, from about 30° to about 90°, from about 30° to about 80°, from about 30° to about 70°, from about 30° to about 60°, from about 30° to about 50°, from about 30° to about 45°, from about 30° to about 40°, from about 40° to about 90°, from about 40° to about 80°, from about 40° to about 70°, from about 40° to about 60°, from about 40° to about 50°, from about 40° to about 45°, from about 45° to about 90°, from about 45° to about 80°, from about 45° to about 70°, from about 45° to about 60°, from about 45° to about 50°, from about 50° to about 90°, from about 50° to about 80°, from about 50° to about 70°, from about 50° to about 60°, from about 60° to about 90°, from about 60° to about 80°, from about 60° to about 70°, from about 70° to about 90°, from about 70° to about 80°, from about 80° to about 90°, about 0, 5, 10, 15, 20, 25, 30, 35, 40, 45, 50, 60, 70, 80, or 90° and any range or value therebetween. Exemplary bent applicators are shown in FIGS. 14A-14B.

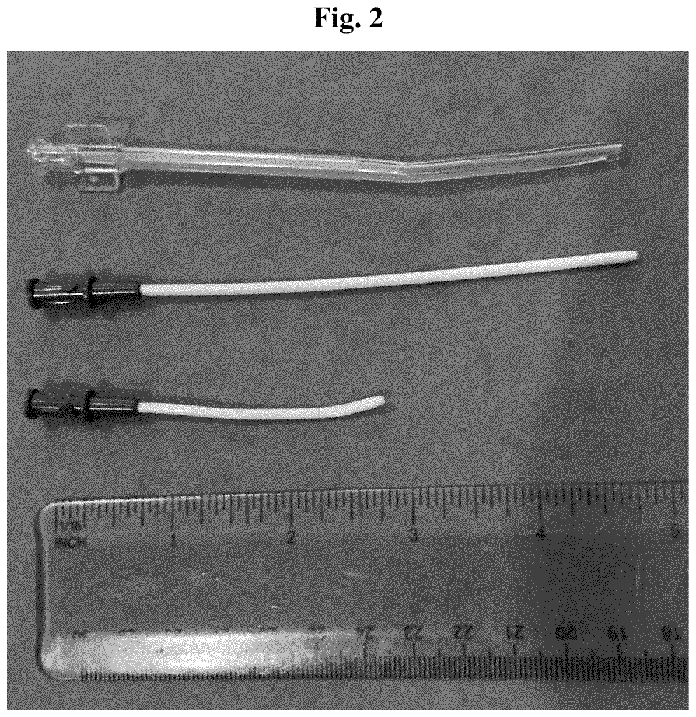

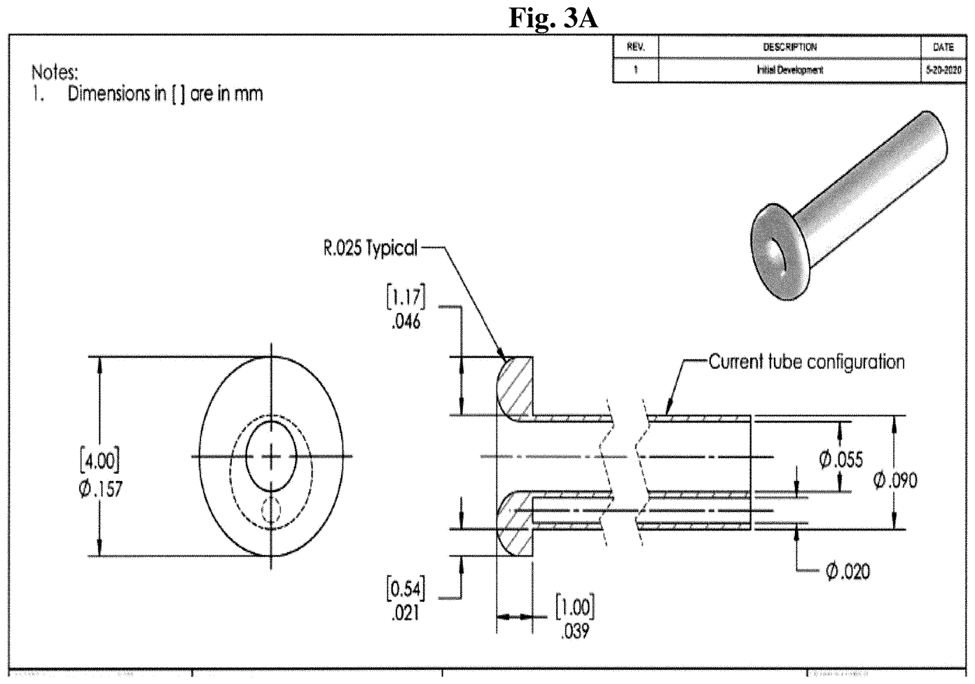

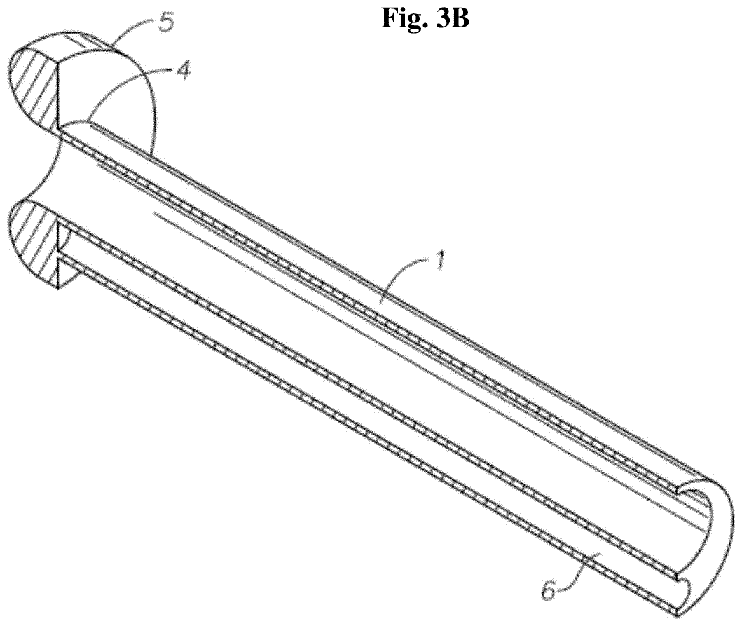

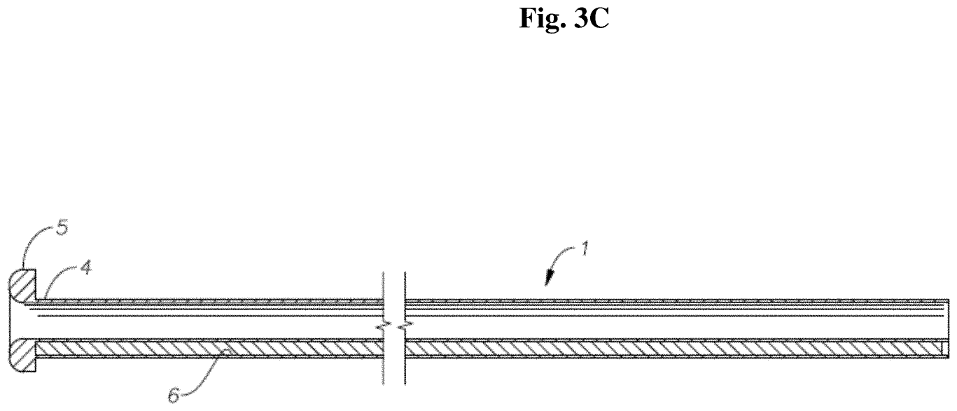

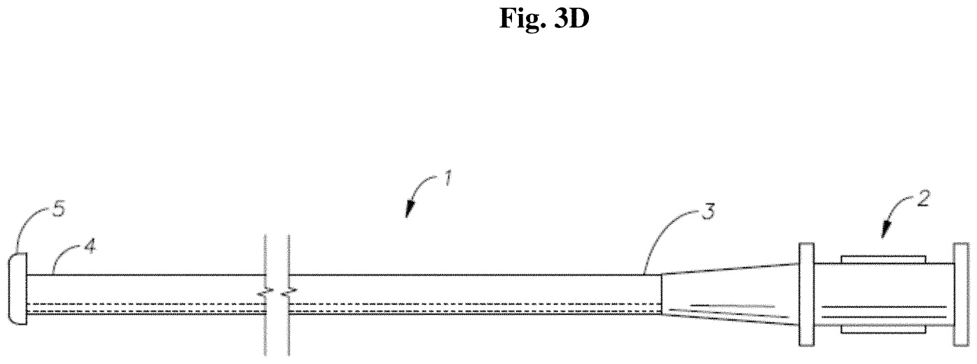

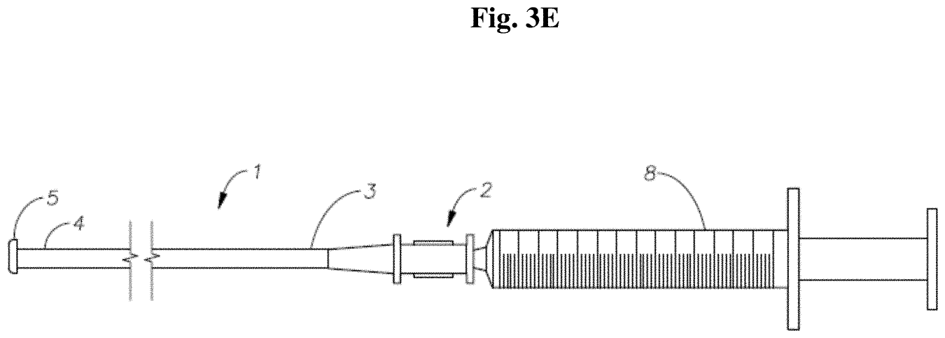

Exemplary devices and portions thereof of the present disclosure are shown in FIGS. 2-3E. As shown in FIG. 2, various embodiments of the device are shown which include a length of tubing 1 and a connector 2 for attachment of a syringe to the length of tubing at the first end of the tubing 3, the tip with the arcuate shape is not shown in FIG. 2. As shown in FIGS. 3A-3C, the length of tubing 1 can include the tip 5 disposed at the second end of the length of tubing 4 which has an arcuate shape. The structural support element 6 can provide bendability to the device by supporting the bent shape of the flexible device. It should be understood that the structural support element can be in the form of a wire or other shape or can be throughout the circumference of the tubing or in any configuration sufficient to impart the requisite rigidity, yet bendability of the device. As shown in FIG. 3D, the device can also have a connector 2 attached to the first end of the device 3. The connector can permit the connection of a syringe 8 or other vessel to the device as shown in FIG. 3E.

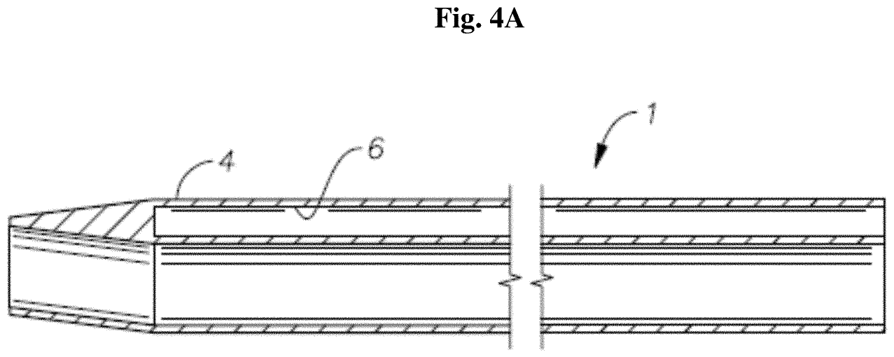

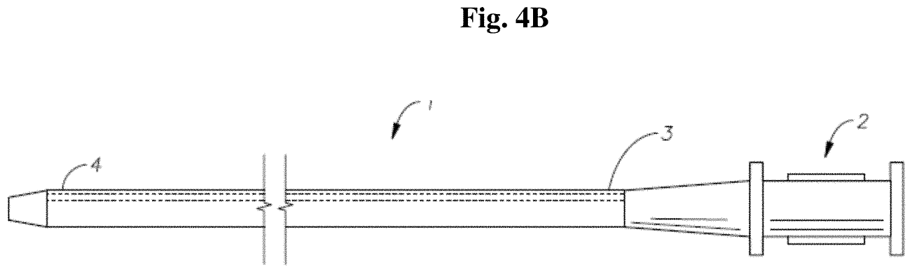

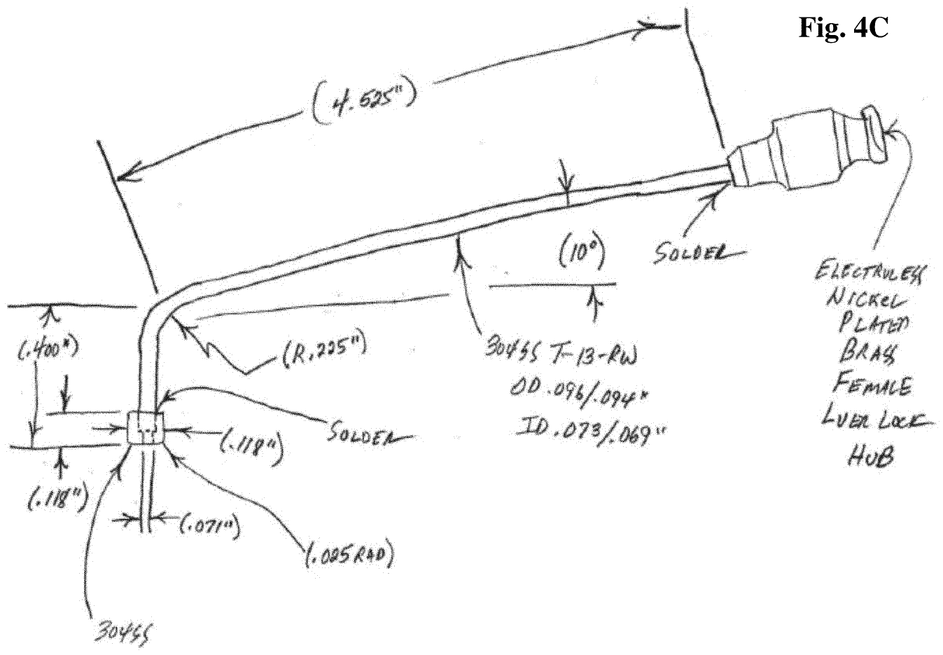

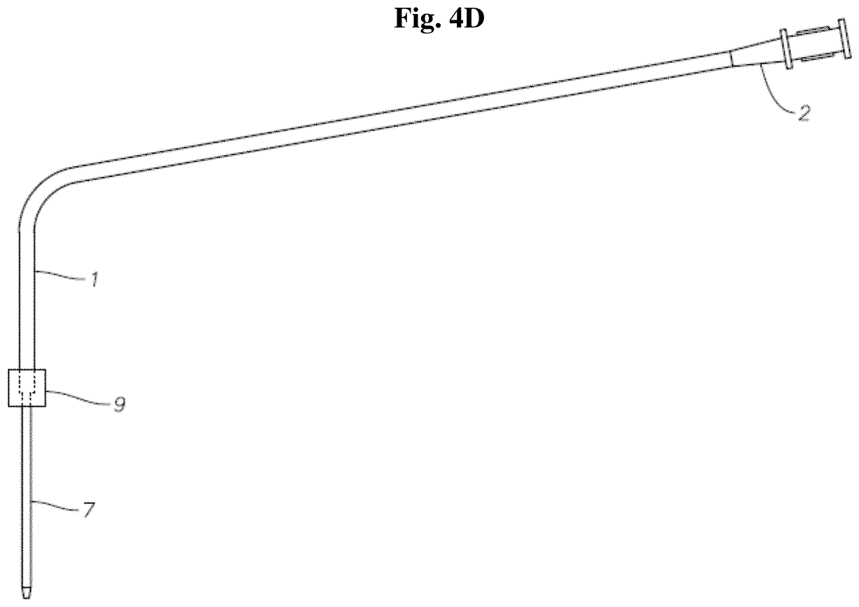

As shown in FIGS. 4A-4D, devices can also be formed of a rigid construction. FIGS. 4A-4B show a rigid applicator device which includes the length of tubing 1 and can, optionally include the structural support element 6 to provide rigidity if the tubing is not sufficiently rigid on its own, and can include a tapered tip, however, any opening to the tip can be used. The device can also include the first end 3 and second end 4 as well as the connector 2 for connection to a syringe or other vessel to the device at the connector (not shown). FIG. 4C shows a hand drawing of a bent, rigid applicator which includes, as shown in FIG. 4D, the connector 2 which is attached to the length of tubing 1 which include a bend and is attached by a fitting or solder 9 to a second length of tubing 7 which has a smaller diameter than the length of tubing 1.

### Kits

The present disclosure provides for kits which include a device for applying a composition of the present disclosure and a cream composition of the present disclosure. In some embodiments, the device is a device of any of the foregoing embodiments. In some embodiments, the cream composition is a composition of any of the foregoing embodiments. Where the device does not already include a syringe or other vessel holding the composition, the kit can include a syringe containing the composition in addition to the device.

In any of the foregoing kit embodiments, the syringe or other vessel holding the composition can include about 0.1 g to about 20 g of the composition. By way of example, but not limitation, the syringe or other vessel holding the composition can include about 0.1 g to about 20 g, about 0.5 g to about 20 g, about 1 g to about 20 g, about 2 g to about 20 g, about 5 g to about 20 g, about 10 g to about 20 g, about 0.1 to about 5 g, about 0.1 g to about 2.5 g, about 0.1 g to about 1 g, about 0.1 to about 0.5 g, about 0.5 g to about 12 g, 0.5 g to about 10 g, about 0.5 g to about 5 g, about 0.5 to about 2.5 g, about 0.5 g to about 1 g, about 1 to about 12 g, about 1 g to about 10 g, about 1 g to about 5 g, about 1 g to about 2 g, about 2 g to about 12 g, about 2 g to about 10 g, about 2 g to about 5 g, about 2 g to about 4 g, about 4 g to about 12 g, about 4 g to about 10 g, about 4 g to about 8 g, about 4 g to about 5 g, about 5 g to about 12 g, about 5 g to about 10 g, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.25, 4.5, 4.75, 5, 5.25, 5.5, 5.75, 6, 6.5, 7, 7.5, 8, 8.5, 9, 9.5, 10, 10.5, 11, 11.5, 12, 13, 14, 15, 16, 17, 18, 19, or 20 g or any range or value therebetween. By way of further example, but not limitation, the syringe or other vessel can contain total of about 0.01 mg to about 3 g of the steroid, such as the amounts in Table 1 by 0.17 g, 0.34 g, 0.7 g, 1 g, 1.4 g, 2 g, 2.1 g, 3 g, 4 g, 4.2 g, 5 g, 6 g, 8 g, 10 g or 20 g. By way of further example, but not limitation, the syringe or other vessel can contain a total amount of steroid of about 0.01 mg to about 3 g, about 0.1 mg to about 3 g, about 0.5 mg to about 3 g, about 1 mg to about 3 mg, about 1.5 to about 3 mg, 0.01 mg to about 1.5 g, about 0.01 mg to about 1 g, about 0.01 mg to about 500 mg, about 0.01 mg to about 250 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 1.5 g, about 0.02 mg to about 1 g, about 0.02 mg to about 500 mg, about 0.02 mg to about 250 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 1.5 g, about 0.03 mg to about 1 g, about 0.03 mg to about 500 mg, about 0.03 mg to about 250 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 1.5 g, about 1 mg to about 1 g, about 1 mg to about 500 mg, about 1 mg to about 250 mg, about 1 mg to about 100 mg, about 1 mg to about 10 mg, about 1 mg to about 5 mg, about 2 mg to about 1.5 g, about 2 mg to about 1 g, about 2 mg to about 500 mg, about 2 mg to about 250 mg, about 2 mg to about 100 mg, about 2 mg to about 10 mg, about 2 mg to about 5 mg, about 8 mg to about 1.5 g, about 8 mg to about 1 g, about 8 mg to about 500 mg, about 8 mg to about 250 mg, about 8 mg to about 100 mg, about 8 mg to about 10 mg, about 10 mg to about 1.5 g, about 10 mg to about 1 g, about 10 mg to about 500 mg, about 10 mg to about 250 mg, about 10 mg to about 100 mg, about 100 mg to about 1.5 g, about 100 mg to about 1 g, about 100 mg to about 500 mg, about 100 mg to about 250 mg, about 250 mg to about 1.5 g, about 250 mg to about 1 g, about 250 mg to about 500 mg, about 500 mg to about 1.5 g, about 500 mg to about 1 g, about 1 g to about 1.5 g, about 0.01 mg, 0.02 mg, 0.05 mg, 0.1 mg, 0.125 mg, 0.15 mg, 0.2 mg, 0.25 mg, 0.3 mg, 0.4 mg, 0.5 mg, 0.6 mg, 0.7 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 220 mg, 240 mg, 250 mg, 260 mg, 280 mg, 300 mg, 330 mg, 350 mg, 360 mg, 390 mg, 400 mg, 440 mg, 450 mg, 480 mg, 500 mg, 550 mg, 600 mg, 650 mg, 700 mg, 750 mg, 800 mg, 850 mg, 900 mg, 950 mg, 1 g, 1.1 g, 1.2 g, 1.3 g, 1.4 g, or 1.5 g, 1.6 g, 1.7 g, 1.8 g, 1.9 g, 2 g, 2.1 g, 2.2 g, 2.3 g, 2.4 g, 2.5 g, 2.6, g, 2.7 g, 2.8 g, 2.9 g, or 3 g or any range or value therebetween. By way of still further example, but not limitation, the syringe or vessel can include between about 0.01 mg to about 500 mg of the agent with antimicrobial activity. By way of example, but not limitation, the syringe or other vessel can contain a total of about 0.01 mg to about 500 mg, about 0.1 mg to about 500 mg, about 1 mg to about 500 mg, about 5 mg to about 500 mg, about 10 mg to about 500 mg, about 100 mg to about 500 mg, about 200 mg to about 500 mg, about 300 mg to about 500 mg, about 400 mg to about 500 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 100 mg, about 1 mg to about 10 mg, about 1 mg to about 5 mg, about 2 mg to about 100 mg, about 2 mg to about 10 mg, about 2 mg to about 5 mg, about 8 mg to about 100 mg, about 8 mg to about 10 mg, about 10 mg to about 100 mg, about 50 mg to about 200 mg, about 50 mg to about 100 mg, about 100 mg to about 200 mg, about 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, or 500 mg or any range or value therebetween.

### Methods of Treatment or Using Devices of the Present Disclosure

In some embodiments, a method for treating a disease or condition of the nasal, sinonasal or nasopharyngeal tissues can include the step of administering a composition of the present disclosure topically to the nasal, sinonasal or nasopharyngeal tissue of the subject. In other embodiments, a method for treating a disease or condition of the otic tissues can include the step of administering a composition of the present disclosure topically to the otic tissue of the subject. In still other embodiments, a method for treating a disease or condition of a mucosal tissue can include the step of administering a composition of the present disclosure topically to the mucosal tissue.

In any of the foregoing embodiments for methods of treatment, the step of applying the composition can be performed as a single administration, which, in some instances, is sufficient to provide an effective treatment of a disease or condition of the nasal, sinonasal or nasopharyngeal tissues. In certain other embodiments, the step of applying the cream composition is performed only once per, by way of example but not limitation, every 10-21 days, every 21-30 days, every 30 to 60 days, every 60 to 90 days, every 90 days to 180 days, or every 180 days to 365 days. It should be understood that a “single administration” in most instances refers to sequential bilateral administration via intranasal administration, external ear canal, middle ear, the eye or other tissues. In some embodiments, the step of applying the cream composition is performed no more than twice per, by way of example but not limitation, 21, 30, 60, 90, 180, or 365 days. In some embodiments, the composition is administered daily or weekly.

In any of the foregoing embodiments for method of treatment, the amount of the composition administered can be an effective amount. In methods for the treatment of a mucosal tissue generally, the composition can contain a therapeutically active ingredient that is suitable for treating the disease or condition of the mucosal tissue. By way of example, but not limitation, a glaucoma drug can be administered to the eye by a composition of the present disclosure. In some embodiments, the mucosal tissue can be nasal, sinonasal, nasopharyngeal, otic, ophthalmic, vaginal, rectal or urethral. It should be understood that, by way of example, but not limitation, inflammation can be treated in these tissues by the compositions of the present disclosure.

Where the tissue to be treated is the eye, the tissues of the eye can, by way of example but not limitation, include the anterior border of the eyelid, bulbar conjunctiva, caruncula lacrimals, ciliar body and muscle, conjunctiva, cornea, eyebrow, eyelids, iris, later angle of the eye, lateral palpebral commissure, lens, lower eyelid, macula, medial angle of the eye, optic nerve, posterior border of the eyelid, pupil, retina, sclera, superior palpebral sulcus, retinal blood vessels, upper eyelid, or vitreous body. Where the disease or condition is of the eye, it can include, by way of example but not limitation, glaucoma, diabetic retinopathy, macular degeneration, uveitis, retinopathy, retinoblastoma, dry eye, disorders of the eye, lacrimal system, orbit, conjunctiva, sclera, cornea, iris, ciliary body, lens, choroid, retina, chorioretinal inflammation, retinal detachments and breaks, retinal vascular occlusions and retinal disorders generally.

In any of the foregoing embodiments for methods of treatment, the amount of cream composition applied will vary based on the extent of the size of the area of the diseased tissue and the size of the patient. In some embodiments, the composition can be administered in an amount of from about 0.5 cubic centimeters (cc) to about 15 cc per intranasal application or a total application amount to the diseased sinus tissue of from about 1 cc to about 10 cc, but more commonly from about 2 cc to about 4 cc per intranasal application or a total application amount to the diseased tissue of the sinus mucosa from about 4 cc to about 8 cc. By way of example, but not limitation, the amount of the composition administered per intranasal or otic application can be about 0.5 cc, 0.75 cc, 1 cc, 1.25 cc, 1.5 cc, 1.75 cc, 2 cc, 2.25 cc, 2.5 cc, 2.75 cc, 3 cc, 3.25 cc, 3.5 cc, 3.75 cc, 4 cc, 4.5 cc, 5 cc, 6 cc, 7 cc, 8 cc, 9 cc, 10 cc, 11 cc, 12 cc, 13 cc, 14 cc, or 15c. It should be understood that for total bilateral application to the disease sinus mucosa, these recited amounts are doubled unless otherwise stated.

In other embodiments, where the composition is administered to a nasal, sinonasal or nasopharyngeal tissue, the composition can be administered in an amount from about 0.5 grams (g) to about 10 g per intranasal administration or a total application amount to the diseased tissue (bilaterally) of from about 1 g to about 20 g, but more commonly from about 2 g to about 4 g per intranasal administration or a total application amount to the diseased tissue of from about 4 g to about 8 g. By way of example, but not limitation, the amount of the composition applied can be about 0.5 g, 0.75 g, 1 g, 1.25 g, 1.5 g, 1.75 g, 2 g, 2.25 g, 2.5 g, 2.75 g, 3 g, 3.25 g, 3.5 g, 3.75 g, 4 g, 4.5 g, 5 g, 6 g, 7 g, 8 g, 9 g, or 10 g per intranasal administration. It should be understood that for total bilateral application to the diseased sinus mucosa, these recited amounts are doubled unless otherwise stated.

Thus, in some embodiments, the amount of steroid administered per nasal, sinonasal or nasopharyngeal tissue, can be a total of about 0.01 mg to about 1.5 g of the steroid. By way of example, but not limitation, the amounts in Table 1 can be multiplied by 0.5 g, 1 g, 2 g, 3 g, 4 g, 5 g, 6 g, 7 g, 8 g, 9 g, or 10 g. By way of further example, but not limitation, the amount of steroid administered per tissue can be a total of 0.01 mg to about 1.5 g, about 0.01 mg to about 750 mg, 0.01 mg to about 500 mg, about 0.01 mg to about 250 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 1.5 g, about 0.02 mg to about 750 mg, about 0.02 mg to about 1 g, about 0.02 mg to about 500 mg, about 0.02 mg to about 250 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 1.5 g, about 0.03 mg to about 750 mg, about 0.03 mg to about 500 mg, about 0.03 mg to about 250 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 1.5 g, about 1 mg to about 750 mg, about 1 mg to about 500 mg, about 1 mg to about 250 mg, about 1 mg to about 100 mg, about 1 mg to about 10 mg, about 1 mg to about 5 mg, about 2 mg to about 1.5 g, about 2 mg to about 750 mg, about 2 mg to about 500 mg, about 2 mg to about 250 mg, about 2 mg to about 100 mg, about 2 mg to about 10 mg, about 2 mg to about 5 mg, about 8 mg to about 1.5 g, about 8 mg to about 750 mg, about 8 mg to about 500 mg, about 8 mg to about 250 mg, about 8 mg to about 100 mg, about 8 mg to about 10 mg, about 10 mg to about 1.5 g, about 10 mg to about 750 mg, about 10 mg to about 500 mg, about 10 mg to about 250 mg, about 10 mg to about 100 mg, about 100 mg to about 1.5 g, about 100 mg to about 750 mg, about 100 mg to about 500 mg, about 100 mg to about 250 mg, about 250 mg to about 1.5 g, about 250 mg to about 750 mg, about 250 mg to about 500 mg, about 500 mg to about 750 mg, about 500 mg to about 1.5 g, about 1 g to about 1.5 g, about 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 220 mg, 240 mg, 250 mg, 260 mg, 280 mg, 300 mg, 330 mg, 350 mg, 360 mg, 390 mg, 400 mg, 440 mg, 450 mg, 480 mg, 500 mg, 550 mg, 600 mg, 650 mg, 700 mg, 750 mg, 800 mg, 850 mg, 900 mg, 950 mg, 1 g, 1.1 g, 1.2 g, 1.3 g, 1.4 g, or 1.5 g or any range or value therebetween. It should be understood that in such embodiments, where bilateral application is applied, these amounts would be doubled.

Thus, in some embodiments, the total amount of the agent with antimicrobial activity administered per nasal, sinonasal or nasopharyngeal tissue can be between about 0.01 mg to about 200 mg of the agent with antimicrobial activity. By way of example, but not limitation, the amount of agent with antimicrobial activity delivered can be a total of about 0.01 mg to about 200 mg, about 0.01 mg to about 100 mg, about 0.01 mg to about 10 mg, about 0.01 mg to about 5 mg, about 0.01 mg to about 1 mg, about 0.01 mg to about 0.1 mg, about 0.02 mg to about 200 mg, about 0.02 mg to about 100 mg, about 0.02 mg to about 10 mg, about 0.02 mg to about 5 mg, about 0.02 mg to about 1 mg, 0.02 mg to about 0.2 mg, about 0.03 mg to about 200 mg, about 0.03 mg to about 100 mg, about 0.03 mg to about 10 mg, about 0.03 mg to about 5 mg, about 0.03 mg to about 1 mg, about 0.03 mg to about 0.3 mg, about 1 mg to about 200 mg, about 1 mg to about 100 mg, about 1 mg to about 10 mg, about 1 mg to about 5 mg, about 2 mg to about 200 mg, about 2 mg to about 100 mg, about 2 mg to about 10 mg, about 2 mg to about 5 mg, about 8 mg to about 200 mg, about 8 mg to about 100 mg, about 8 mg to about 10 mg, about 10 mg to about 200 mg, about 10 mg to about 100 mg, about 50 mg to about 200 mg, about 50 mg to about 100 mg, about 100 mg to about 200 mg, about 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, or 200 mg or any range or value therebetween. It should be understood that in such embodiments, bilateral application is used, these amounts would be doubled.

In other embodiments, where the composition is administered to an otic tissue, the composition can be administered in an amount from about 0.1 g to about 3 g per ear. By way of example, but not limitation, the composition can be administered in an amount from about 0.1 g to about 2.1 g, about 0.17 g to about 2.1 g, about 0.1 g to about 1 g, about 1 g to about 2.5 g, about 1 g to about 2 g, about 0.1 g, 0.17 g, 0.2 g, 0.3 g, 0.4 g, 0.5 g, 0.6 g, 0.7 g, 0.8 g, 0.9 g, 1 g, 1.1 g, 1.2 g, 1.3 g, 1.4 g, 1.5 g, 1.6 g, 1.7 g, 1.8 g, 1.9 g, 2 g, 2.1 g, 2.2 g, 2.3 g, 2.4 g, 2.5 g, 2.6 g, 2.7 g, 2.8 g, 2.9 g, or 3 g or any range or value therebetween, preferably about 0.7 g. It should be understood that for total bilateral application to the diseased otic tissue, these recited amounts are doubled unless otherwise stated. In some embodiments, the total amount of steroid delivered to the ear tissue is between about 0.01 mg and about 500 mg. By way of example, but not limitation, the total amount of steroid delivered to the ear tissue can be between about 0.01 mg to about 500 mg, about 0.01 mg to about 250 mg, about 0.01 mg to about 100 mg, about 0.1 mg to about 50 mg, about 0.01 mg to about 10 mg, about 0.01 mg to about 5 mg, about 0.01 mg to about 1 mg, about 0.1 mg to about 500 mg, about 0.1 mg to about 250 mg, about 0.1 mg to about 100 mg, about 0.1 mg to about 50 mg, about 0.1 mg to about 10 mg, about 0.1 mg to about 5 mg, about 0.1 mg to about 1 mg, about 0.5 mg to about 500 mg, about 0.5 mg to about 250 mg, about 0.5 mg to about 100 mg, about 0.5 mg to about 50 mg, about 0.5 mg to about 10 mg, about 0.5 mg to about 5 mg, about 0.5 mg to 1 mg, about 1 mg to about 500 mg, about 1 mg to 250 mg, about 1 mg to 100 mg, about 1 mg to about 50 mg, about 1 mg to about 10 mg, about 1 mg to about 5 mg, about 5 mg to about 500 mg, about 5 mg to about 250 mg, about 5 mg to about 100 mg, about 5 mg to about 50 mg, about 5 mg to about 10 mg, about 10 mg to about 500 mg, about 10 mg to about 250 mg, about 10 mg to about 100 mg, about 10 mg to about 50 mg, about 50 mg to about 500 mg, about 50 mg to about 250 mg, about 50 mg to about 100 mg, about 100 mg to about 500 mg, about 100 mg to about 250 mg, about 250 mg to about 500 mg, about 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 75 mg, 100 mg, 150 mg, 200 mg, 250 mg, 275 mg, 300 mg, 325 mg, 350 mg, 375 mg, 400 mg, 425 mg, 450 mg, 475 mg, or 500 mg or any value or range therebetween. It should be understood that these amounts are per ear and would be doubled for bilateral administration.

In some embodiments, the total amount of the agent with antimicrobial activity delivered to the ear tissue is between about 0.01 mg and about 100 mg. By way of example, but not limitation, the total amount of the agent with antimicrobial activity delivered to the ear tissue can be between about 0.01 mg to about 100 mg, 0.01 mg to about 50 mg, about 0.01 mg to about 10 mg, about 0.01 mg to about 5 mg, about 0.01 mg to about 1 mg, about 0.1 mg to about 100 mg, about 0.1 mg to about 50 mg, about 0.1 mg to about 10 mg, about 0.1 mg to about 5 mg, about 0.1 mg to about 1 mg, about 0.5 mg to about 100 mg, about 0.5 mg to about 50 mg, about 0.5 mg to about 10 mg, about 0.5 mg to about 5 mg, about 0.5 mg to 1 mg, about 1 mg to about 100 mg, about 1 mg to about 50 mg, about 1 mg to about 10 mg, about 1 mg to about 5 mg, about 5 mg to about 100 mg, about 5 mg to about 50 mg, about 5 mg to about 10 mg, about 10 mg to about 100 mg, about 10 mg to about 50 mg, about 50 mg to about 100 mg, about 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 60 mg, 70 mg, 80 mg, 90 mg, or 100 mg or any value or range therebetween. It should be understood that these amounts are per ear and would be doubled for bilateral administration.

It should likewise be understood that these amounts can be adjusted depending on the mucosal tissue to be treated. By way of example, but not limitation, where tissues are to be treated, the total amount of composition administered can be about 0.01 g to about 10 g. By way of further example, but not limitation, the amount administered to the tissue can be about 0.01 g to about 0.1 g, about 0.02 g to about 0.1 g, about 0.03 g to about 0.1 g, about 0.04 g to about 0.1 g, about 0.05 g to about 0.1 g, about 0.1 g to about 1 g, about 0.1 g to about 2 g, about 1 g to about 5 g, about 1 g to about 10 g, about 2 g to about 5 g, about 2 g to about 10 g, about 5 g to about 10 g, about 0.01, 0.02, 0.03, 0.04, 0.05, 0.06, 0.07, 0.08, 0.09, 0.1, 0.015, 0.2, 0.25, 0.3, 0.35, 0.4, 0.45, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 g or any range or value therebetween.

In any of the foregoing embodiments for methods of treatment, the disease or condition can be the result of a gram negative bacteria, a gram positive bacteria, a fungus, a yeast or can be polymicrobial including a combination of bacteria, fungi and/or yeast. In other embodiments, the disease or condition can be the result of inflammation with no identified microbial infection.

In any of the foregoing embodiments for methods of treatment, where the disease or condition is of the nasal, sinonasal or nasopharyngeal tissues, the compositions of the present disclosure can be used to treat various conditions of the nasal, sinonasal and nasopharyngeal tissues. In any of the foregoing embodiments, the disease or conditions can be inflammation of the nasal, sinonasal or nasopharyngeal tissues. By way of example, but not limitation, such conditions of the nasal, sinonasal and nasopharyngeal tissues can include disease, infections, symptoms and combinations thereof. By way of example but not limitation, such diseases or infections can include sinus, edema, chronic sinusitis, acute sinusitis, mucormycosis, polymicrobial sinusitis, nasal polyps, bacterial sinusitis, allergic fungal sinusitis, chronic bacterial sinusitis, chronic allergic fungal sinusitis, rhinosinusitis and the like. By way of further example, but not limitation, such diseases and infections can include sinus edema, acute sinusitis, acute sinusitis infection, acute sinusitis bacterial infection, acute sinusitis viral infection, acute rhinosinusitis, ageusia, allergic fungal sinusitis, anosmia, bacterial sinusitis, barosinusitis, barotrauma, chronic polyposis, chronic bacterial sinusitis, chronic allergic fungal sinusitis, chronic sinusitis, chronic recurrent sinusitis, chronic recurrent sinusitis infection, chronic recurrent sinusitis bacterial infection, chronic recurrent sinusitis viral infection, chronic rhinosinusitis, chronic rhinosinusitis with polyps, chronic rhinosinusitis without polyps, chronic recurrent rhinosinusitis, central compartment atopic disease, cystic fibrosis, diffuse sinusitis, diffuse type 2 sinusitis, eosinophilic rhinosinusitis, fungal sinusitis, granulomatosis with polyangiitis, maxillary sinus infection, mucormycosis, nasal polyps, non-eosinophilic rhinosinusitis, non-eosinophilic chronic rhinosinusitis, paranasal sinus retention cysts, polymicrobial sinusitis, recurrent rhinosinusitis, recurrent acute rhinosinusitis, rhinosinusitis, sinusitis, sinonasal polyps, and sphenoid sinus infection. By way of still further example, but not limitation, methods of the present disclosure can be used for treating the following sinus symptoms: the need to blow the nose, nasal blockage, sneezing, runny nose, cough, post-nasal discharge, thick nasal discharge, ear fullness, dizziness, ear pain, facial pain or pressure, decreased sense of smell or taste, difficulty falling asleep, waking up at night, lack of a good night's sleep, waking up tired, fatigue, reduced productivity, reduced concentration, frustration, restlessness or irritability, sadness, embarrassment, and combinations thereof. In some embodiments, the condition further includes the need to blow the nose, nasal blockage, sneezing, runny nose, cough, post-nasal discharge, thick nasal discharge, ear fullness, dizziness, ear pain, facial pain or pressure, decreased sense of smell or taste, difficulty falling asleep, waking up at night, lack of a good night's sleep, waking up tired, fatigue, reduced productivity, reduced concentration, frustration, restlessness or irritability, sadness, embarrassment, or a combination thereof. Thus, these sinus symptoms can occur in conjunction with a disease, infection or other condition or can be conditions to be treated themselves. In some embodiments, a subject has previously undergone functional endoscopic sinus surgery (FESS). In some embodiments, a subject has previously undergone sinonasal surgery. In some embodiments, the subject after having undergone FESS has thereafter developed a chronic inflammatory response. In some embodiments, the subject has undergone FESS and developed chronic allergic fungal sinusitis. In some embodiments, a subject for which the present compositions and methods is useful is suffering from chronic allergic fungal sinusitis after FESS. In some embodiments, the patient is experiencing an exacerbation of symptoms after a period of mild or no symptoms after FESS with or without the use of nasal steroid sprays, oral antibiotics and/or nasal irrigations. In some embodiments, a subject has had FESS resulting in abnormal nasal tissue, described as hypertrophic, inflammatory, and granulation type tissue. In a further aspect of these embodiments, the subject's post-FESS sinusitis was treated with nasal steroid sprays, oral antibiotics and/or nasal irrigations for a period of a year with minimal to no change in disease state prior to performance of the present methods. In some embodiments, the subject is suffering from chronic sinus inflammation as a result of a bacterial infection. In some embodiments, the methods of the present disclosure can be performed at the time of FESS. In some embodiments, the patient has not previously undergone FESS. In some embodiments, the methods of the present disclosure can be performed during balloon sinus dilation. In some embodiments, the compositions of the present disclosure can be administered at the time of FESS. In some embodiments, the compositions of the present disclosure can be administered during balloon sinus dilation. Even in the instance that the chronic inflammation is the result of a bacterial infection, cream compositions comprising clotrimazole may be useful as this active agent has been shown to have antibacterial activity in addition to its antimycotic activity against both gram-positive and gram-negative microorganisms. Specifically, clotrimazole has been shown to result in a reduction in Pseudomonas aeruginosa and to have antibacterial activity against Streptococci, Staphylococci, Gardnerella vaginalis, and Corynebacteria. However, as discussed in further detail below, other antibiotic active agents can be substituted in the cream composition of the present disclosure. In some embodiments, the patient has no detectable microbial infection. In other embodiments, the patient has a detectable microbial infection, such as bacterial or fungal infection. Thus, the compositions and methods of the present disclosure can be useful in the absence or presence of detectable microbial infection. In some embodiments, the condition can include a bacterial infection. In some embodiments, the condition is at least partially the result of a bacterial infection and a biofilm has formed on the surface of the sinonasal or nasopharyngeal tissue. In some embodiments, the condition can include a fungal infection. In some embodiments, the condition can include a yeast infection. In some embodiments, the condition can include a polymicrobial infection. In any of the foregoing embodiments for methods of treatment, where the disease or condition is of the nasal, sinonasal or nasopharyngeal tissues, the composition can be administered to the maxillary sinus, frontal sinus, ethmoid sinus, sphenoid sinus, maxillary mucosa, frontal mucosa, ethmoid mucosa, sphenoid mucosa, turbinates, nasal passage, nasolacrimal duct, nasal cavity and nasal tissue.

In any of the foregoing embodiments for methods of treatment, where the disease or condition is of the otic tissues, the compositions of the present disclosure can be used to treat various conditions of the otic tissues. By way of example, but not limitation, such conditions of the otic tissues can include disease, infections, symptoms and combinations thereof. By way of example but not limitation, such diseases or infections can include otitis externa such as, by way of example, but not limitation, acute diffuse bacterial external otitis (swimmer's ear), acute localized external otitis (furunculosis), impetigo of the external ear, erysipelas, perichondritis, chronic external otitis, otomycosis, malignant otitis externa, herpes, tube otorrhea, choleastome, and otitis media with perforation. By way of further example, but not limitation, such diseases or infections can include acute otitis media, acute localized external otitis (furunculosis), acute mastoiditis, acoustic neuroma, auditory processing disorder, autoimmune inner ear disease, benign paroxysmal positional vertigo, barotrauma, choleasteatoma, chronic external otitis, chronic otitis media, chronic otitis media with effusion, dizziness, erysipelas, herpes zoster otitis, hearing loss, infectious myringitis, inner ear infection, inner ear related vertigo, labyrinthitis, malignant otitis externa, Meniere's disease, middle ear infection, otitis media, otitis media with effusion, otitis media with perforation, otitis externa, otomycosis, outer ear infection, perforated eardrum, perichondritis, recurrent vestibulopathy, serous otitis media, superior semicircular canal dehiscence syndrome, tinnitus, tube otorrhea, vertigo, vestibulopathy, vestibular neuritis, and viral labyrinthitis. It should be understood that other otic conditions can be treated with the compositions of the present invention. In any of the foregoing embodiments, the otic tissue can be the auricle, cochlea, ear canal, Eustachian tube, external auditory canal, inner ear, middle ear, outer ear, round window, semicircular canals, tympanic membrane, tympanic cavity, metal tissue or hair cells.

In any of the foregoing embodiments for methods of treatment, the composition can be administered in an effective amount. In any of the foregoing embodiments for methods of treatment, the composition of the present disclosure can be applied to the tissue using a device of the present disclosure. By way of example, but not limitation, a syringe containing a composition of the present disclosure can be attached to a device of the present disclosure via a connector and the tip of the device can be inserted into the nose or ear to apply the composition to the target tissue. It should be understood that the step of administering the cream composition can be performed using other suitable devices that allow for application of the composition to the target tissue. In some embodiments, the device can be guided by an endoscope.

In some embodiments, a method of treatment of a disease or condition of a nasal, sinonasal or nasopharyngeal tissue or an otic tissue, can include using a device of the present disclosure to administer a composition to the tissue, where the composition is suitable for treating the disease or condition. In such embodiments, the composition need not be limited to a composition of the present disclosure. In such embodiments, the composition can be administered in an effective amount. It should be understood that, to the extent such compositions are suitable for treating the diseases and conditions of these tissues, they can be applied using a device of the present disclosure.

The pharmaceutical compositions and the methodologies for their application, will now be described with reference to the following non-limiting examples.

### EXAMPLES

The following examples are provided for exemplary and illustrative purposes and are not intended to otherwise limit the scope of the present disclosure.

### Example 1: Manufacturing & Physical Stability Testing of Cream Formulations

Cream formulations provided in Table 4 below, were prepared according to the method outlined in FIG. 1A.

TABLE 4
Cream Formulations (amounts provided as % by weight)

| | Lot Number | | | |
|---|---|---|---|---|
| Ingredient | 2019-10-8 | 2019-10-3 | 2019-10-4 | 2020-01-C |
| Purified Water | QS | QS | QS | QS |
| | (~18.5) | (~78.9) | (~81.6) | (~62.9) |
| Polysorbate 80 | 5 | 5 | 2.5 | |
| Propylene Glycol | | | | 15 |
| Glycerin | 2.5 | | | |
| Betamethasone | 0.0322 | 0.0322 | 0.0322 | 0.0322 |
| Diproprionate | | | | |
| Clotrimazole | 1 | 1 | 1 | 0.5 |
| Polyoxyl 40 Stearate | 1 | 1 | 1 | |
| Cetyl Alcohol | 1 | 1 | 1 | |
| Glyceryl Monostearate | 0.5 | 0.5 | 0.5 | |
| Petrolatum | 46.2997 | 8 | 8 | |
| Mineral Oil | 23.14985 | | | |
| Benzyl Alcohol | 0.75 | 0.9 | 0.75 | 0.5 |
| Oleth-2 | | 3 | 3 | |
| Isopropyl Myristate | | | | |
| Carbopol 980 | 0.25 | 0.6 | 0.6 | 0.5 |
| 1% NaOH | pH to 5 | pH to 5 | pH to 5 | pH to 5 |
| Cetostearyl Alcohol | | | | 6 |
| Cetyl Esters Wax | | | | 1.5 |
| Sorbitan Monostearate | | | | 2.5 |
| 2-Octyldodecanol | | | | 6.75 |
| Polysorbate 60 | | | | 3.75 |

Briefly, for a 250 g batch size, to generate the aqueous (water) phase, approximately 125-150 g of water was placed in a 400 mL beaker with a 4-blade propeller at approximately ½ the height of the liquid. In the case of 2019-10-8, glycerin was added and mixing was performed at 200-300 rpm to dissolve the glycerin. The mixing propeller was lower and the speed increased to approximately 800 rpm for 1 minute. The mixer was then turned off and Carbopol was added by sprinkling a layer across the layer of the solution followed by pulsing 2-5 times to wet and disperse the Carbopol, with the process being repeated until all of the Carbopol was added. The mixture was then mixed for 30 minutes at 800-1000 rpm with rotating the beaker every 5-10 minutes. If necessary to adjust the pH, a dilute sodium hydroxide solution (about 1%) was added (pH 4, ˜0 grams; pH 5, ˜20-25 grams; pH 6, ˜35-40 grams, pH 7, ˜45-55 grams) while mixing at 1000 rpm. The mixture was then Q.S. to volume with water and mixed for about 30 minutes. At 200-300 rpm, polysorbate 80 was then added and the mixture mixed for approximately 45 minutes with raising and lowering the beaker every 5-10 minutes and foaming avoided.

To prepare the oil phase, all remaining ingredients not used to prepare the aqueous phase except for the clotrimazole, betamethasone dipropionate and benzyl alcohol were added in order from liquid to most solid in a 250 mL beaker with a stir bar. The mixture was heated on a hot plate to 65+/−5° C. with mixing for approximately 15 minutes until most of the solids were melted (Settings: 80° C.; 100-350 rpm). Stirring was slowed to 50-100 rpm at a setting of 75° C. for approximately 10 minutes until the mixture was homogenous.

A disk impeller blade was added to the aqueous phase vessel and mixing was performed at the lowest speed for approximately 5 minutes. The aqueous phase was then heated to 62+/−3° C. with the highest rate of mixing that did not cause foaming (approximately 1200⁺ rpm) with waiting for about 30 minutes during heating. The agent with antimicrobial activity—clotrimazole—and steroid—betamethasone dipropionate—were added to each of the aqueous phase preparation and the oil phase preparation with approximately half of the clotrimazole and betamethasone dipropionate added to each of the aqueous phase (with mixing turned off) and the oil phase, respectively. Each phase was then mixed for an additional 10-15 minutes.

The blade in the 400 mL beaker was adjusted to higher than ½ of the liquid height and the speed of mixing was increased to approximately 1800 rpm to apply high shear. The oil phase was added to the aqueous phase while the oil phase was still hot. Stirring was continued for approximately 45 minutes with raising and lowering the mixing blade every 5-10 minutes. Benzyl alcohol was added under high shear at approximately 1800 rpm. The mixture was mixed at approximately 1200 rpm for about 30 minutes with raising and lowering the mixing blade every 5-10 minutes. Water was added to account for evaporation and the mixture was mixed for approximately 10 minutes.

The resulting creams were packaged into syringes that were then capped.

The resulting creams, as packaged, were autoclaved at 110° C. for 10 minutes or at 130° C. for 3 minutes.

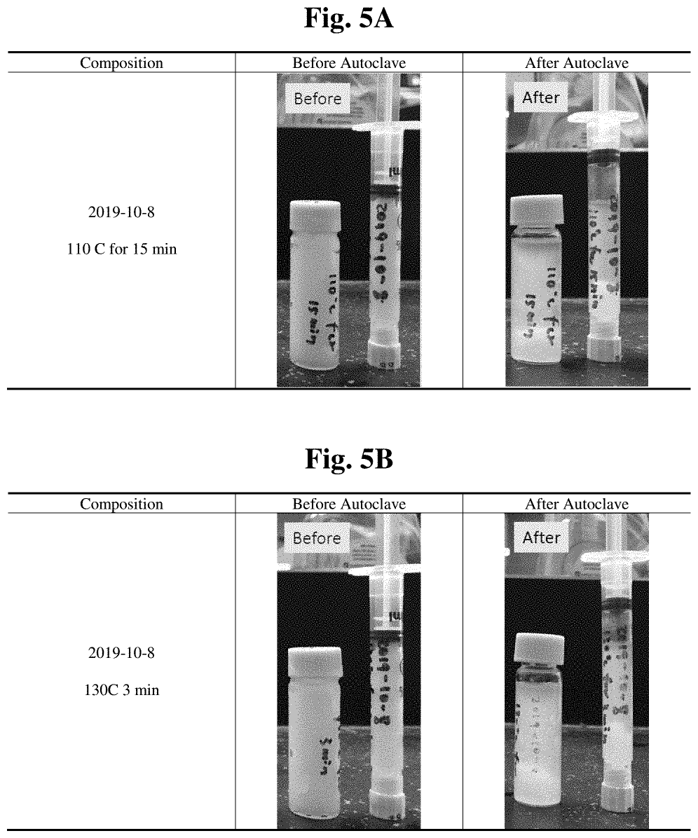

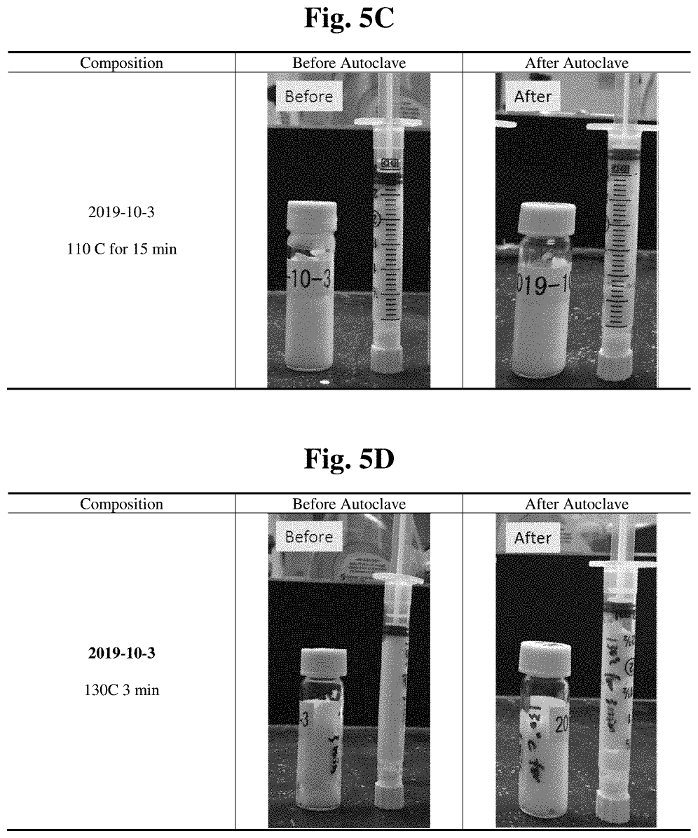

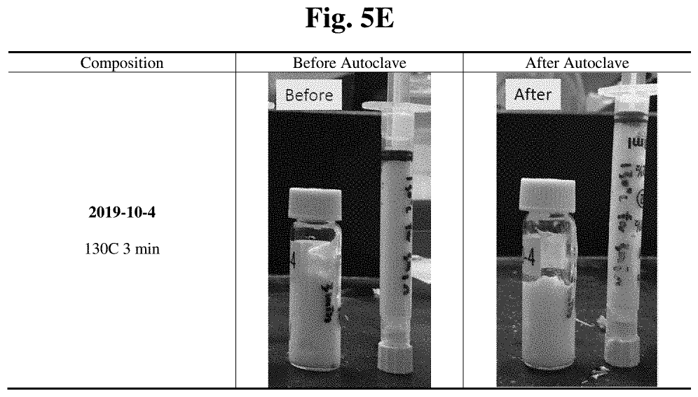

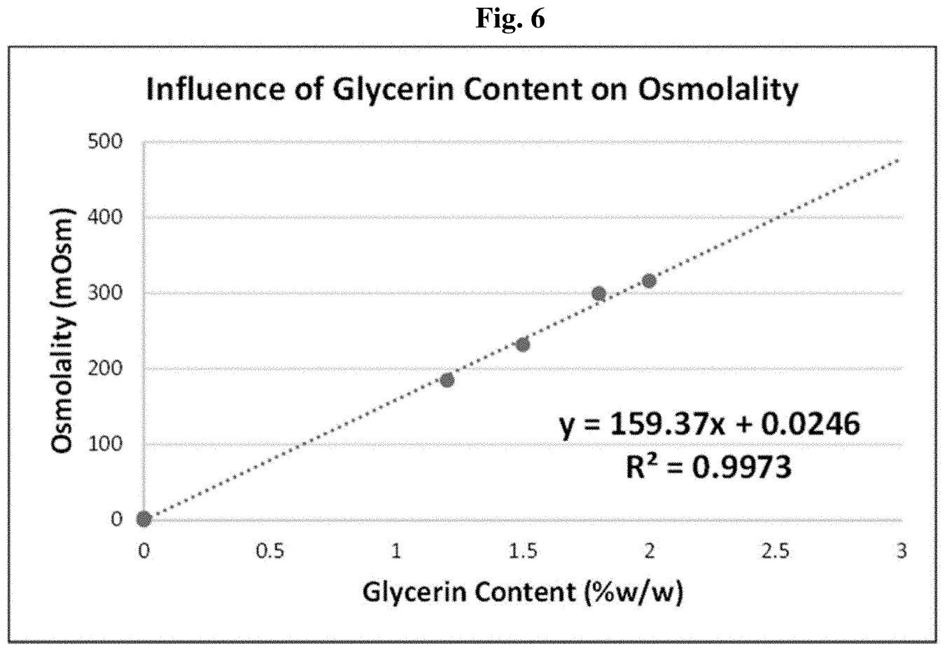

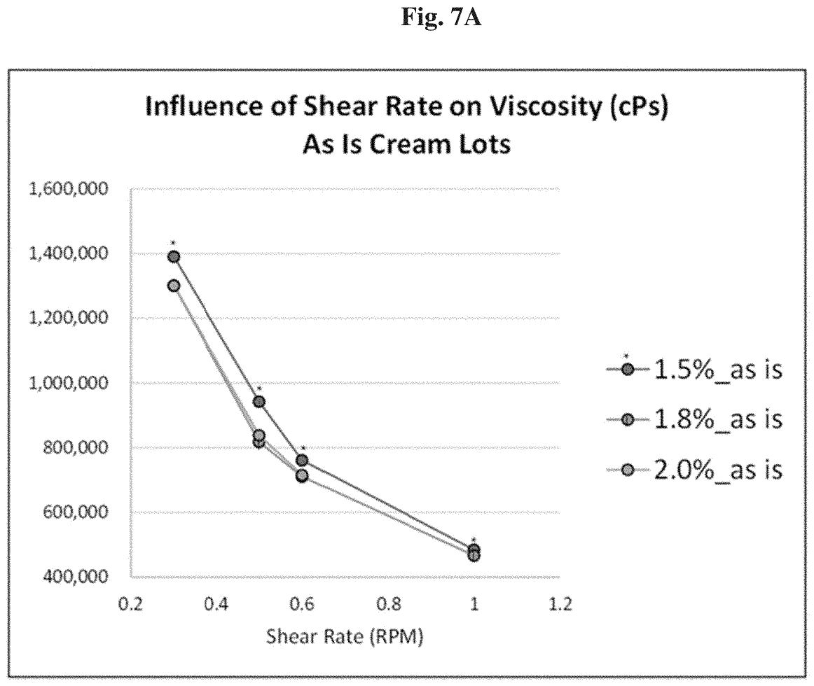

The creams were assessed for physical stability after autoclaving by visual inspection. Photographs of the autoclaved creams are shown in FIGS. 5A-5E. As shown, Composition 2019-10-8 did not retain physical stability and separated into 2 phases under both autoclave conditions. Composition 2019-10-3 did retain physical stability and did not separate into 2 phases under either autoclave condition. The absence of two distinct phases was confirmed by globule size measurement as described in Example 3. Composition 2019-10-4 also retained physical stability and did not separate into 2 phases under either autoclave condition. The absence of two distinct phases was likewise confirmed by globule size measurement as described in Example 3. 2020-01-C partially separated (not shown in FIGS. 5A-5E).

### Example 2: Assessment of Tonicity in Compositions Containing Glycerin

Compositions 2019-11-1, 2019-11-2, 2019-11-3 and 2019-11-4 were prepared as described above. The formulations of these compositions are shown in Table 5 below.

TABLE 5
Cream Formulations (amounts provided as % by weight)

| | Lot Number | | | |
|---|---|---|---|---|
| Ingredient | 2019-11-1 | 2019-11-2 | 2019-11-3 | 2019-11-4 |
| Clotrimazole | 1.0 | 1.0 | 1.0 | 1.0 |
| Betamethasone | 0.0322 | 0.0322 | 0.0322 | 0.0322 |
| dipropionate | | | | |
| Polysorbate 80 | 5.0 | 5.0 | 5.0 | 5.0 |
| Glycerin | 1.5 | 1.2 | 1.8 | 2.0 |
| Carbopol 980 | 0.6 | 0.6 | 0.6 | 0.6 |
| Polyoxy1 40 Stearate | 1.0 | 1.0 | 1.0 | 1.0 |
| Cetyl Alcohol | 1.0 | 1.0 | 1.0 | 1.0 |
| Glyceryl Monostearate | 0.5 | 0.5 | 0.5 | 0.5 |
| Petrolatum | 8.0 | 8.0 | 8.0 | 8.0 |
| Oleth-2 | 3.0 | 3.0 | 3.0 | 3.0 |
| Benzyl Alcohol | 0.9 | 0.9 | 0.9 | 0.9 |
| 1% NaOH | pH to 5 | pH to 5 | pH to 5 | pH to 5 |
| Purified Water | QS | QS | QS | QS |
| | (~77.4) | (~77.7) | (~77.1) | (~76.9) |
| Osmolality | 231 | 185 | 299 | 316 |
| (mOsm/kg) | | | | |

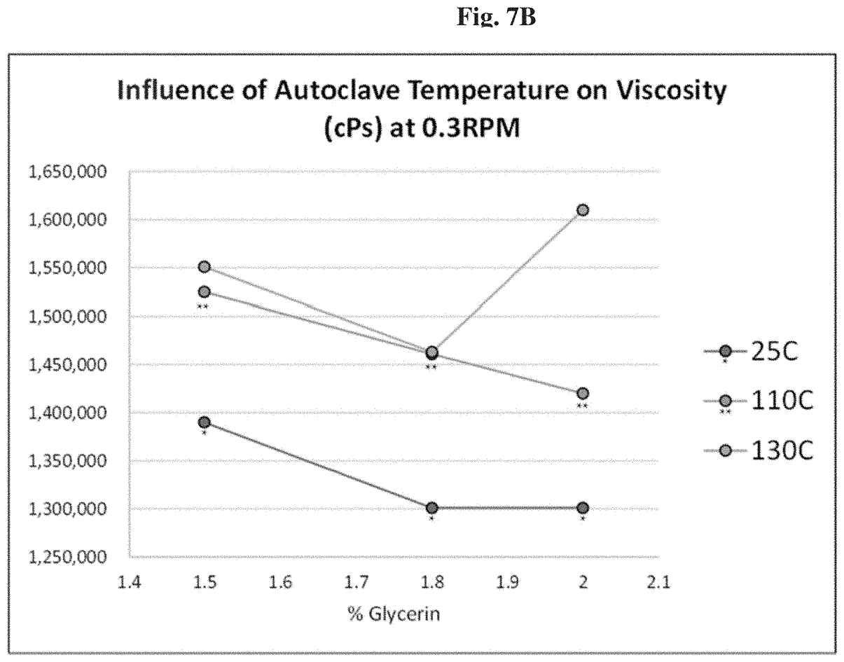

The osmolality of each composition was measured using a Precision Systems Microosmette Model 5004 or equivalent. The microosmette was calibrated per the manufacturer instructions. Cream composition samples were prepared by weighing about 1 g of cream into each of 3, 15 mL conical tubes followed by 3 g, 5 g, or 10 g of Milli-Q water into each tube, respectively. The samples were vortexed at 2000 rpm for at least 30 seconds and centrifuged at 1800G for 45 minutes. Sample osmolality was measured according to manufacturer instructions. To calculate osmolality, the mean osmolality measurements were plotted (y-axis) against the weight fractions of cream (amount of cream in each sample per the total weight of the sample) (x-axis) and the slope obtained, if linear, was determined to be the osmolality of the undiluted cream. As shown in FIG. 6, the osmolality of the compositions varied linearly with glycerin content. Thus, glycerin content can be used to modulate the tonicity of the formulation.

### Example 3: Globule Size and Particle Size Determination for Compositions

In cream formulations, such as where the cream is an oil-in-water emulsion, the active ingredients—clotrimazole and betamethasone dipropionate—may not completely dissolve and some particles of these ingredients are “suspended” within the cream matrix. In addition, the oil droplets dispersed in the aqueous phase are referred to as “globules.”

The size and distribution of the suspended particles and the globules can be measured using a static microscopic image analyzer (Malvern Morphologi G3S). The size distribution is determined and the “Dn10, Dn50 and Dn90” indicate the size at which 10%, 50% and 90% of the particles within the distribution are smaller than on a number basis. Thus, Dn50=2 μm means that 50% of the particles are smaller than 2 μm on a number basis.

Similarly, the “Dv10, Dv50 and Dv90” indicate the size at which 10%, 50% and 90% of the particles within the distribution are smaller than on a volume basis. Thus, Dv50=2 μm means that 50% of the particles are smaller than 2 μm on a volume basis.

A Number Mean and Volume Mean size are also reported. The particle shape is determined, and the aspect ratio and circularity are reported.

Creams are thermodynamically unstable, due to the large increase in surface energy that results from the combination of interfacial tension, the large surface area of the dispersed phase and the density differences of the two phases. Droplets of the internal phase can coalesce with a considerable reduction in surface free energy. Thus, creams tend to separate—the less dense phase rises and the denser phase falls. When exposed to heat, the homogenously distributed droplets begin to aggregate and ultimately coalesce into large globules and the cream becomes unstable, with phase separation typically occurring. Accordingly, measurement of globule size is stability indicating. Maintenance of globule size after a cream has been exposed to heat and other stress conditions, such as autoclaving, demonstrates the cream is stable.

Table 6 provides compositions that were assessed for particle globule size distribution and particle size distribution which were prepared by the methods as described in Example 1 (2020-01 C refers to the “Control” formulation).

TABLE 6
Cream Formulations (amounts provided as % by weight)

| | Lot Number | | |
|---|---|---|---|
| | | 2020-01-1 | |
| | | “pH 5 | |
| | 2019-11-3 | with | 2020-01-C |
| Ingredient | “pH 5” | EDTA” | “Control” |
| Clotrimazole | 1.00 | 1.00 | See composition in |
| Betamethasone | 0.0322 | 0.0322 | Table above |
| dipropionate | | | |
| Polysorbate 80 | 5.00 | 5.00 | |
| Glycerin | 1.80 | 1.75 | |
| EDTA Disodium | | 0.05 | |
| Carbopol 980 | 0.60 | 0.60 | |
| Polyoxyl 40 Stearate | 1.00 | 1.00 | |
| Cetyl Alcohol | 1.00 | 1.00 | |
| Glyceryl | 0.50 | 0.50 | |
| Monostearate | | | |
| Petrolatum | 8.00 | 8.00 | |
| Oleth-2 | 3.00 | 3.00 | |
| Benzyl Alcohol | 0.90 | 0.90 | |
| 1% NaOH | pH to 5 | pH to 5 | |
| Purified Water | QS | QS | |
| | (~77.1) | (~77.1) | |

Table 7 below provides the globule size distribution (as reported in microns, μm) for the formulations in Table 6 before (“As Is”) and after Autoclaving. Surprisingly, the compositions of the present inventions did not separate and the globule size was maintained.

TABLE 7
Globule Size Distribution (as reported in microns, um)

| | | 2020-01-1 | |
|---|---|---|---|
| | 2019-11-3 | “pH 5 with | 2020-01-C |
| | “pH 5” | EDTA” | “Control” |
| As Is | | | |
| Dn10 | 1.51 | 1.70 | 1.13 |
| Dn50 | 2.84 | 2.67 | 2.02 |
| Dn90 | 4.95 | 4.35 | 3.04 |
| Number Mean | 3.12 | 2.91 | 2.11 |
| Autoclave | | | |
| 110° C. for 10 minutes | | | |
| Dn10 | 0.96 | 1.23 | Separated |
| Dn50 | 1.46 | 1.79 | |
| Dn90 | 2.14 | 2.66 | |
| Number Mean | 1.54 | 1.91 | |
| Autoclave | | | |
| 130° C. for 1 minutes | | | |
| Dn10 | | 1.26 | Separated |
| Dn50 | | 1.71 | |
| Dn90 | | 2.43 | |
| Number Mean | | 1.83 | |
| Autoclave | | | |
| 130° C. for 3 minutes | | | |
| Dn10 | | 1.33 | Separated |
| Dn50 | | 1.87 | |
| Dn90 | | 2.54 | |
| Number Mean | | 1.93 | |
| Autoclave | | | |
| 130° C. for 5 minutes | | | |
| Dn10 | | 0.97 | Separated |
| Dn50 | | 1.48 | |
| Dn90 | | 2.24 | |
| Number Mean | | 1.58 | |

Particle growth or “Ostwald ripening” of suspended particles is also a destabilizing process, resulting from temperature fluctuations during storage. Temperature fluctuations may change particle size distribution if the solubility of the drug is temperature dependent. For example, if the temperature is raised, undissolved drug crystals may dissolve and form a supersaturated solution, which favor crystal growth on cooling. As the dissolved drug crystallizes out of solution, it will preferentially occur on the surface of a crystal in the suspension.

Table 8 provides the particle size distribution (as reported in microns, μm) for the formulations in Table 6 before (“As Is”) and after Autoclaving. Surprisingly, compositions of the present invention maintained their particle size and particle size growth was not observed.

TABLE 8
Particle Size Distribution (as reported in microns, um)

| | | 2020-01-1 |
|---|---|---|
| | 2019-11-3 | “pH 5 with |
| | “pH 5” | EDTA” |
| As Is | | |
| Dn10 | 1.17 | 1.15 |
| Dn50 | 2.54 | 2.13 |
| Dn90 | 4.85 | 4.13 |
| Number Mean | 2.91 | 2.46 |
| Autoclave | | |
| 110° C. for 10 minutes | | |
| Dn10 | 0.87 | 1.18 |
| Dn50 | 1.54 | 1.80 |
| Dn90 | 2.71 | 2.96 |
| Number Mean | 1.72 | 1.99 |
| Autoclave | | |
| 130° C. for 1 minutes | | |
| Dn10 | | 1.25 |
| Dn50 | | 1.71 |
| Dn90 | | 2.55 |
| Number Mean | | 1.87 |
| Autoclave | | |
| 130° C. for 3 minutes | | |
| Dn10 | | 1.12 |
| Dn50 | | 1.94 |
| Dn90 | | 3.03 |
| Number Mean | | 2.05 |
| Autoclave | | |
| 130° C. for 5 minutes | | |
| Dn10 | | 0.94 |
| Dn50 | | 1.65 |
| Dn90 | | 2.83 |
| Number Mean | | 1.82 |

For comparison purposes, the globule size distribution and particle size distribution (each in microns, μm) for two commercial products that have not been autoclaved are provided in Tables 9 and 10.

TABLE 9
Globule Size Distribution (as reported in microns, um)

| | Clotrimazole and | |
|---|---|---|
| | Betamethasone | |
| | Dipropionate Cream, | Clotrimazole Vaginal |
| Name | USP, 1%/0.05% | Cream, USP, 1% |
| Manufacturer | NorthStar | Sunmark |
| NDC | 16714-496-01 | 49348-793-76 |
| Lot # | 323547 | E14JA |
| As Is | | |
| Dn10 | 0.33 | 0.39 |
| Dn50 | 0.78 | 1.14 |
| Dn90 | 1.52 | 2.43 |
| Number Mean | 0.88 | 1.34 |

TABLE 10
Particle Size Distribution (as reported in microns, um)

| | Clotrimazole and | |
|---|---|---|
| | Betamethasone | |
| | Dipropionate Cream, | Clotrimazole Vaginal |
| Name | USP, 1%/0.05% | Cream, USP, 1% |
| Manufacturer | NorthStar | Sunmark |
| NDC | 16714-496-01 | 49348-793-76 |
| Lot # | 323547 | E14JA |
| As Is | | |
| Dn10 | 7.71 | 0.44 |
| Dn50 | 11.74 | 0.91 |
| Dn90 | 18.97 | 1.81 |
| Number Mean | 12.72 | 1.07 |

### Example 4: Chemical Stability During Sterilization

Representative lots prepared as described in the previous examples were packaged into 4 different configurations and sterilized by autoclave or gamma irradiation (15 kGy dose). Sample descriptions and autoclave conditions are described in the tables below. Cream was packaged into either Becton Dickenson syringes (rubber plunger), NormJect syringes (polyethylene plunger) or Scintillation vials (glass) for the study. One series of samples was packaged into Scintillation vials and 5 rubber stoppers to allow the cream to come into intimate contact with the rubber. These different packaging configurations were selected to investigate the influence of rubber plungers and syringe materials on the chemical stability of the creams.

Samples will be analyzed for chemical degradants of Clotrimazole and Betamethasone before and after the sterilization processes using HPLC. A prednisone internal standard (IS) stock will be prepared by adding about 25 mg of prednisone to a 25 mL volumetric flask filled about ⅔ full with ethanol followed by sonication and fill the flask fully with ethanol to yield a 1000 μg/mL stock solution. The stock solution will be diluted by transferring 4 mL of the stock solution to a 100 mL volumetric flask and diluting to volume with ethanol to yield an internal standard solution. A betamethasone dipropionate stock solution will be prepared similarly, using about 33.4 mg of betamethasone dipropionate in a 25 mL volumetric flask.

A working standard solution will be prepared by combining 1 mL of the internal standard solution and 4 mL of the betamethasone dipropionate stock solution in a 50 mL volumetric flask to which about 167 mg of clotrimazole and about 150 mg of benzyl alcohol were added with the flask having been filled about ⅔ with ethanol. The flask will then be filled to volume with ethanol and mixed well.

A check standard will be prepared by adding about 33.4 mg of clotrimazole to a10 mL volumetric flask filled about ⅔ with methanol which was mixed and then a sufficient volume of methanol to fill the flask was added followed by mixing well.

A clotrimazole related compound A (RCA) stock solution will be prepared by weighing about 21 g RCA into a 25 mL volumetric flask filled about ⅔ with methanol followed by mixing and filling of the flask to volume. Curve solutions will be prepared by adding 8 mL, 5 mL, 4 mL, 5 mL and 1 mL of the RCA stock solution to a 25 mL, 25 mL, 25 mL, 50 mL and 25 mL volumetric flask, respectively, with addition of methanol to volume. Before addition of methanol 1 mL of the IS stock solution will be added to the 5 mL stock into a 50 mL flask. The dilution scheme is shown in Table 11 below:

TABLE 11
RCA Solution Preparation

| | Theoretical | | Dilution | Vol. of |
|---|---|---|---|---|
| RCA Curve | RCA Conc. | Level of RCA | Flask | RCA Stock |
| Solution | (ug/mL) | (vs Clo) | (mL) | (mL) |
| 1 | 268.8 | 8% | 25 mL | 8 mL |
| 2 | 168.0 | 5% | 25 mL | 5 mL |
| 3 | 134.4 | 4% | 25 mL | 4 mL |
| 4* | 84.0 | 2.5% | 50 mL | 5 mL |
| 5 | 33.6 | 1% | 25 mL | 1 mL |

A standard curve for RCA and for the other standard will be created using the HPLC procedure and used to correlate peak area with concentration.

Cream compositions will be prepared for HPLC by weighing 2 g (+/−0.2 g) of cream into 50 mL centrifuge tubes. 3 mL of ethanol will be added to each tube as well as 3 mL of internal standard solution. The tubes will then be vortexed for about 30 seconds to disperse the contents. Samples will then be placed in a 70° C. oven for 15 minutes to dissolve the cream. The samples will then be immediately vortexed for at least 30 seconds. Tubes will then be placed on a room temperature shaker at 400 rpm for 20 minutes. After shaking, the tubes will be centrifuged at 3000G and 4° C. for 30 minutes. The supernatant will then be collected and transferred to 3 mL syringes and filtered, if necessary, for HPLC analysis. The same will be done for corresponding lots of cream without the active ingredients to rule out degradant peaks from inactive ingredient.

HPLC will be performed with a run time of 45 minutes using a 0.5 mL/minute flow rate and as mobile phases: A. Ammonium phosphate buffer, pH 7.0+/−0.1; B. Methanol; and C. Acetonitrile, using the following gradient as shown in Table 12:

TABLE 12
HPLC Gradients

| Time (min) | % A | % B | % C |
|---|---|---|---|
| 0.0 | 63 | 25 | 12 |
| 1.8 | 43 | 45 | 12 |
| 10.8 | 28 | 60 | 12 |
| 22 | 28 | 60 | 12 |
| 23.3 | 30 | 5 | 65 |
| 38.5 | 30 | 5 | 65 |
| 38.6 | 63 | 25 | 12 |
| 45.0 | 63 | 25 | 12 |

The injection volume will be 3 μL, sample temperature will be ambient, detector wavelength will be 254 nm (data collected for information only at 270 nm), column temperature will be 35° C., the column will be a Thermo Hypersil ODS column (150×3 mm, 3 μm), and the guard column was a Thermo ODS guard cartridge (30×3 mm, 3 μm) or equivalent.

The percentage area will be calculated by subtracting the area of the analyte peak from the chromatogram from the total area of the analyte and related degradant peaks from the chromatogram.

### Example 5: pH, Viscosity and Osmometry Testing

The pH of several commercial formulations was measured using standard methods and the results are provided in Table 13 below.

TABLE 13
pH of Commercial Formulations

| Brand | Manufacturer | NDC | Lot | pH |
|---|---|---|---|---|
| Betamethasone | Taro | 51672-1309-3 | E870131687 | 3.8 |
| Dipropionate Gel, | | | | |
| Augmented, 0.05% | | | | |
| Betamethasone | Taro | 51672-1310-3 | L870534014 | 4.6 |
| Dipropionate | | | | |
| Cream, USP, | | | | |
| Augmented, 0.05% | | | | |
| Clotrimazole and | NorthStar | 16714-496-01 | 323547 | 6.2 |
| Betamethasone | | | | |
| Dipropionate | | | | |
| Cream, USP, | | | | |
| 1%/0.05% | | | | |
| Clotrimazole | Sunmark | 49348-793-76 | E14JA | 5.1 |
| Vaginal | | | | |
| Cream, USP, 1% | | | | |
| Betamethasone | Perrigo | 45802-376-32 | 8LT0424 | 3.3 |
| Dipropionate | | | | |
| Cream, USP, | | | | |
| Augmented, 0.05% | | | | |

pH of the cream compositions of the present disclosure was performed on an as-is cream sample after centrifugation at 1000G for 2 minutes (about 2 grams of cream) or on a 1:5 dilution of the cream prepared from about 1 gram of cream in 5 grams of water in a 15 mL conical tube which was then vortexed at 2000 rpm at least 30 seconds until no separation of cream and water was observed.

The viscosity of 3 lots of cream prepared as described in the foregoing Examples was measured using a Brookfield RVDVII+ at 0.3-1 rpm (shear rate) using Spindle 28 and sample chamber and water jacket 13R using the small sample adapter. Viscosity was measured by setting the rotational speed and a torque between 10% and 100%. The viscosity was then read at the different rotational speeds. Viscosity is reported in cPs (centipoise) in Table 14.

TABLE 14
Viscosity Measurements (in cPs)

| | Lot Number & Description | | |
|---|---|---|---|
| | 2019-11-01 | | |
| | “1.5% Glycerin” | | |
| | | 110° C. | 130° C. |
| RPM | As Is | 30 min | 3 min |
| 0.3 | 1,390,000 | 1,525,000 | 1,551,000 |
| 0.5 | 942,000 | 988,000 | |
| 0.6 | 760,800 | | |
| 1 | 485,000 | | |
| | Lot Number & Description | | |
| | 2019-11-03 | | |
| | “1.8% Glycerin” | | |
| | | 110° C. | 130° C. |
| RPM | As Is | 30 min | 3 min |
| 0.3 | 1,301,000 | 1,461,000 | 1,463,000 |
| 0.5 | 818,000 | 977,000 | 939,000 |
| 0.6 | 710,800 | | 805,800 |
| 1 | 467,000 | | |
| | Lot Number & Description | | |
| | 2019-11-04 | | |
| | “2% Glycerin” | | |
| | | 110° C. | 130° C. |
| RPM | As Is | 30 min | 3 min |
| 0.3 | 1,301,000 | 1,420,000 | 1,610,000 |
| 0.5 | 838,000 | 938,000 | |
| 0.6 | 715,800 | 822,500 | |
| 1 | | | |

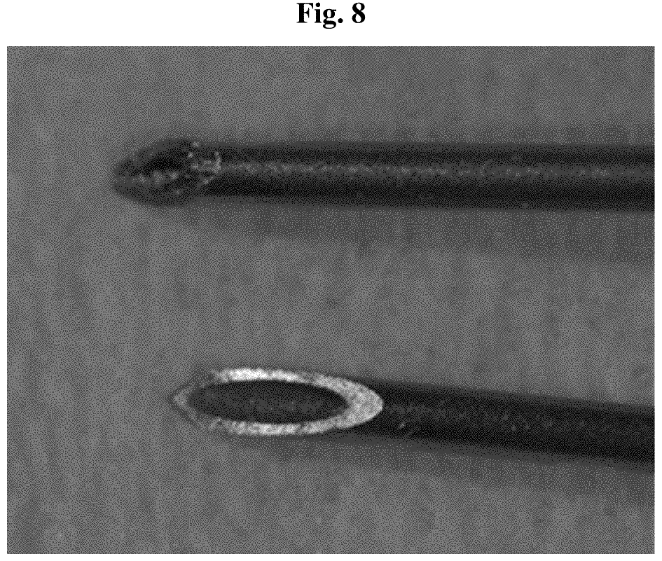

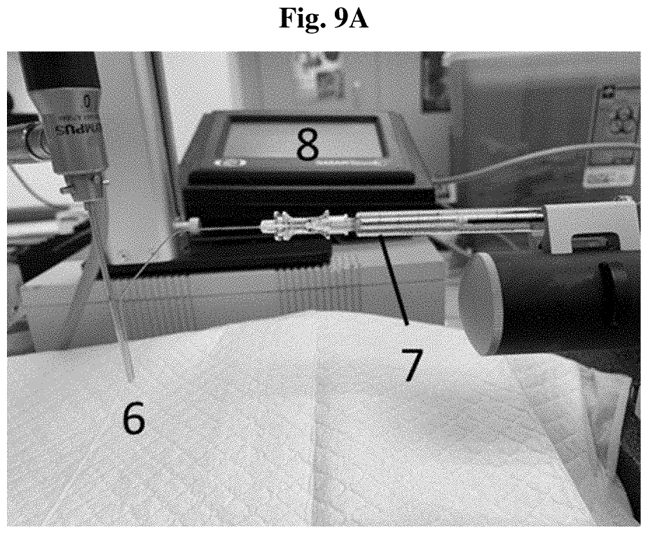

FIGS. 7A and 7B show the effect of shear rate and autoclave temperature, respectively, on the viscosity of the formulations tested. The data in FIG. 7B were obtained at 0.3 rpm.

The viscosity of several commercial formulations was measured using standard methods and the results are provided in Table 15 below.

TABLE 15
Viscosity Measurements of Commercial Products (in cPs)

| | Betamethasone Dipropionate Gel, |
|---|---|
| | Augmented, 0.05% |
| | 2020-07-16 |
| | Taro |
| RPM | As Is |
| 0.3 | 1,158,000 |
| 0.5 | 745,000 |
| 0.6 | 633,300 |
| 1 | 403,500 |

| | Betamethasone Dipropionate Cream, |
|---|---|
| | USP, Augmented, 0.05% |
| | 2020-07-14 |
| | Taro |
| RPM | As Is |
| 0.3 | Out of Range |
| 0.5 | Out of Range |
| 0.6 | 92,500 |
| 1 | 91,000 |
| | Clotrimazole and Betamethasone |
| | Dipropionate Cream, USP, 1%/0.05% |
| | 2020-07-17 |
| | Northstar |
| RPM | As Is |
| 0.3 | 275,000 |
| 0.5 | 149,000 |
| 0.6 | 124,100 |
| 1 | 93,500 |

| | Clotrimazole Vaginal Cream, USP, 1% |
|---|---|
| | 2020-07-18 |
| | Sunmark |
| RPM | As Is |
| 0.3 | 275,000 |
| 0.5 | 165,000 |
| 0.6 | 148,300 |
| 1 | 127,500 |

| | Betamethasone Dipropionate Cream, |
|---|---|
| | USP, Augmented, 0.05% |
| | 2020-07-15 |
| | Perrigo |
| RPM | As Is |
| 0.3 | 788,300 |
| 0.5 | 494,000 |
| 0.6 | 411,600 |
| 1 | 264,000 |

The osmolality of several commercial formulations was measured using standard methods and the results are provided in Table 16 below.

TABLE 16
Osmolality of Commercial Products (in mOsmol/kg)

| Brand | Manufacturer | NDC | Lot | Osmolality |
|---|---|---|---|---|
| Betamethasone Dipropionate Gel, | Taro | 51672-1309-3 | E870131687 | 9,735 |
| Augmented, 0.05% | | | | |
| Betamethasone Dipropionate | Taro | 51672-1310-3 | L870534014 | 1,456 |
| Cream, USP, Augmented, 0.05% | | | | |
| Clotrimazole and Betamethasone | NorthStar | 16714-496-01 | 323547 | 1,582 |
| Dipropionate Cream, USP, | | | | |
| 1%/0.05% | | | | |
| Clotrimazole Vaginal Cream, USP, | Sunmark | 49348-793-76 | E14JA | 153 |
| 1% | | | | |
| Betamethasone Dipropionate | Perrigo | 45802-376-32 | 8LT0424 | 1,308 |
| Cream, USP, Augmented, 0.05% | | | | |

Additional compositions were prepared as described in the Examples based on the following formulations:

TABLE 17
Additional Cream Formulations

| | Lot Number | | | |
|---|---|---|---|---|
| Ingredient | 2020-07-04 | 2020-07-05 | 2020-07-06 | 2020-07-07 |
| Clotrimazole | 1.0 | 1.0 | 1.0 | 1.0 |
| Betamethasone | 0.0322 | 0.0322 | 0.0322 | 0.0322 |
| dipropionate | | | | |
| Polysorbate 80 | 5.0 | 5.0 | 5.0 | 5.0 |
| Glycerin | 1.75 | 1.75 | 1.75 | 1.75 |
| Carbopol 980 | 0.6 | 0.6 | 0.6 | 0.6 |
| Polyoxyl 40 Stearate | 1.0 | 1.0 | 1.0 | 1.0 |
| Cetyl Alcohol | 1.0 | 1.0 | 1.0 | 1.0 |
| Glyceryl Monostearate | 0.5 | 0.5 | 0.5 | 0.5 |
| Petrolatum | 8.0 | 8.0 | 8.0 | 8.0 |
| Oleth-2 | 3.0 | 3.0 | 3.0 | 3.0 |
| Benzyl Alcohol | 0.9 | 0.9 | 0.9 | 0.9 |
| Disodium EDTA | 0.0 | 0.0 | 0.0 | 0.0 |
| 1% NaOH | pH to 4 | pH to 5 | pH to 5 | pH to 6 |
| | | | After | |
| | | | emulsification | |
| Purified Water | QS | QS | QS | QS |

| | Lot Number | | | |
|---|---|---|---|---|
| Ingredient | 2020-07-08 | 2020-07-09 | 2020-07-10 | 2020-07-11 |
| Clotrimazole | 1.0 | 1.0 | 1.0 | 1.0 |
| Betamethasone | 0.0322 | 0.0322 | 0.0322 | 0.0322 |
| dipropionate | | | | |
| Polysorbate 80 | 5.0 | 5.0 | 5.0 | 5.0 |
| Glycerin | 1.75 | 1.75 | 1.75 | 1.75 |
| Carbopol 980 | 0.6 | 0.6 | 0.6 | 0.6 |
| Polyoxyl 40 Stearate | 1.0 | 1.0 | 1.0 | 1.0 |
| Cetyl Alcohol | 1.0 | 1.0 | 1.0 | 1.0 |
| Glyceryl Monostearate | 0.5 | 0.5 | 0.5 | 0.5 |
| Petrolatum | 8.0 | 8.0 | 8.0 | 8.0 |
| Oleth-2 | 3.0 | 3.0 | 3.0 | 3.0 |
| Benzyl Alcohol | 0.9 | 0.9 | 0.9 | 0.9 |
| Disodium EDTA | 0.05 | 0.05 | 0.05 | 0.05 |
| 1% NaOH | pH to 4 | pH to 5 | pH to 5.5 | pH to 6 |
| Purified Water | QS | QS | QS | QS |

| | Lot Number | |
|---|---|---|
| Ingredient | 2020-07-12 | 2020-07-13 |
| Clotrimazole | 1.0 | 1.0 |
| Betamethasone | 0.0322 | 0.0322 |
| dipropionate | | |
| Polysorbate 80 | 5.0 | 5.0 |
| Glycerin | 1.75 | 1.75 |
| Carbopol 980 | 0.4 | 0.4 |
| Polyoxyl 40 Stearate | 1.0 | 1.0 |
| Cetyl Alcohol | 1.0 | 1.0 |
| Glyceryl Monostearate | 0.5 | 0.5 |
| Petrolatum | 8.0 | 8.0 |
| Oleth-2 | 3.0 | 3.0 |
| Benzyl Alcohol | 0.9 | 0.9 |
| Disodium EDTA | 0.05 | 0.05 |
| 1% NaOH | pH to 5 | pH to 6 |
| Purified Water | QS | QS |

For lot 2020-07-06, the pH was adjusted at the end of manufacturing as described in the present disclosure. The viscosity of each formulation was measured using the Brookfield RVDVII+ as described previously. The viscosity of lot 2020-07-05 was also measured using the cone-and-plate method using a Brookfield Rheometer DV3T CP Rheometer with Spindle CP52 at a torque of 10-100% at 25.0+/−0.1° C. Briefly, 0.5 mL of the formulation was added to the sample cup and the program was run at 0.3 RPM, 0.6 RPM, 1.5 RPM, 3 RPM, 6 RPM, 12 RPM, 30 RPM or 60 RPM. Samples of the formulations were also autoclaved at 110° C. for 10 minutes and the viscosity of the sterile formulations measured using the Brookfield RVDVII+ as described previously.

The results of the viscosity measurements are provided in Table 18 and 19 below.

TABLE 18
Viscosities for Cream Formulations (in cPs)

| | Lot Number | | | |
|---|---|---|---|---|
| RPM | 2020-07-04 | 2020-07-04AC | 2020-07-05 | 2020-07-05AC |
| 0.3 | Δ | 1,321,000 | 938,300 | 1,576,000 |
| 0.5 | | 900,000 | 581,000 | 971,000 |
| 0.6 | | 755,800 | 498,300 | δ |
| 0.8 | | 602,500 | 399,300 | |
| 1 | | δ | 326,500 | |
| 1.5 | | | 232,000 | |
| 2 | | | 180,000 | |
| 2.5 | | | 144,000 | |
| 3 | | | 117,800 | |
| 4 | | | 89,250 | |
| 5 | | | 73,200 | |
| 6 | | | 60,000 | |
| 10 | | | 39,000 | |
| 12 | | | 34,000 | |
| 20 | 2,625 | | 22,970 | |
| 30 | 2,150 | | Δ | |
| 50 | 1,500 | | | |
| 60 | 1,316 | | | |
| 100 | 930 | | | |
| | Lot Number | | | |
| RPM | 2020-07-06 | 2020-07-06AC | 2020-07-07 | 2020-07-07AC |
| 0.3 | 830,000 | δ | 973,300 | 1,205,000 |
| 0.5 | 517,000 | | 584,000 | 789,000 |
| 0.6 | 434,100 | | 498,300 | 683,300 |
| 0.8 | 346,200 | | 400,600 | 543,700 |
| 1 | 286,000 | | 334,500 | 448,000 |
| 1.5 | 209,000 | | 241,300 | 326,600 |
| 2 | 160,700 | | 185,000 | δ |
| 2.5 | 128,600 | | 147,400 | |
| 3 | 105,000 | | 124,300 | |
| 4 | 80,870 | | 95,000 | |
| 5 | 68,600 | | 77,000 | |
| 6 | 57,160 | | 64,750 | |
| 10 | 37,750 | | 44,800 | |
| 12 | 32,200 | | 37,410 | |
| 20 | 22,050 | | 24,400 | |
| 30 | δ | | Δ | |
| 50 | | | | |
| 60 | | | | |
| 100 | | | | |
| | Lot Number | | | |
| RPM | 2020-07-08 | 2020-07-08AC | 2020-07-09 | 2020-07-09AC |
| 0.3 | Δ | 421,600 | 638,300 | 1,041,000 |
| 0.5 | | 253,000 | 421,000 | 663,000 |
| 0.6 | | 210,800 | 365,800 | 560,000 |
| 0.8 | | 158,100 | 290,600 | 438,700 |
| 1 | | 130,000 | 245,000 | 358,500 |
| 1.5 | | 98,000 | 178,600 | 254,300 |
| 2 | | 73,500 | 140,000 | 198,500 |
| 2.5 | | 59,000 | 115,000 | 164,200 |
| 3 | | 56,000 | 103,600 | 142,500 |
| 4 | | 45,500 | 80,250 | 113,500 |
| 5 | | 39,700 | 64,700 | 94,100 |
| 6 | | 33,250 | 53,830 | 76,410 |
| 10 | | 22,300 | 35,300 | δ |
| 12 | | 20,250 | 30,410 | |
| 20 | | 13,900 | 21,900 | |
| 30 | | 10,860 | 16,060 | |
| 50 | 1,110 | 7,680 | δ | |
| 60 | 975 | 6,516 | | |
| 100 | 700 | 4,650 | | |
| | Lot Number | | | |
| RPM | 2020-07-10 | 2020-07-10AC | 2020-07-11 | 2020-07-11AC |
| 0.3 | 603,300 | 793,300 | 763,300 | 715,000 |
| 0.5 | 398,000 | 494,000 | 458,000 | 448,000 |
| 0.6 | 329,100 | 411,600 | 381,600 | 389,100 |
| 0.8 | 274,300 | 328,100 | 293,100 | 309,300 |
| 1 | 227,500 | 273,500 | 235,000 | 256,500 |
| 1.5 | 151,000 | 195,600 | 168,600 | 187,300 |
| 2 | 118,000 | 152,200 | 126,700 | 144,000 |
| 2.5 | 98,400 | 125,200 | 101,200 | 119,400 |
| 3 | 89,830 | 109,100 | 84,500 | 106,600 |
| 4 | 73,620 | 88,370 | 68,250 | 86,250 |
| 5 | 59,800 | 72,400 | 54,700 | 70,500 |
| 6 | 51,830 | 62,500 | 46,000 | 62,750 |
| 10 | 35,300 | 41,350 | 28,900 | 41,400 |
| 12 | 31,290 | 37,040 | 25,200 | 36,080 |
| 20 | 21,050 | 24,300 | 16,970 | 24,520 |
| 30 | 16,210 | δ | 12,760 | δ |
| 50 | δ | | 8,850 | |
| 60 | | | 7,875 | |
| 100 | | | δ | |
| | Lot Number | | | |
| RPM | 2020-07-12 | 2020-07-12AC | 2020-07-13 | 2020-07-13AC |
| 0.3 | 251,600 | 560,000 | 283,300 | 401,600 |
| 0.5 | 171,000 | 340,000 | 183,000 | 248,000 |
| 0.6 | 142,500 | 285,000 | 156,600 | 206,600 |
| 0.8 | 107,500 | 223,100 | 126,800 | 163,700 |
| 1 | 89,000 | 178,500 | 101,500 | 131,000 |
| 1.5 | 68,660 | 131,600 | 79,330 | 99,000 |
| 2 | 51,500 | 98,750 | 59,500 | 74,250 |
| 2.5 | 41,600 | 85,400 | 50,200 | 59,400 |
| 3 | 42,000 | 79,000 | 47,160 | 60,330 |
| 4 | 33,750 | 61,870 | 39,120 | 48,500 |
| 5 | 27,000 | 79,500 | 32,100 | 38,800 |
| 6 | 24,830 | 44,160 | 28,500 | 35,250 |
| 10 | 16,950 | 29,550 | 18,700 | 23,850 |
| 12 | 15,250 | 25,750 | 17,410 | 21,540 |
| 20 | 9,850 | 17,100 | 11,750 | 14,270 |
| 30 | 8,183 | 12,760 | 9,066 | 10,510 |
| 50 | 5,840 | 8,600 | 6,530 | 7,400 |
| 60 | 5,183 | 7,516 | 5,850 | 6,366 |
| 100 | 3,795 | Δ | 4,325 | 4,390 |

*AC = autoclaved;
Δ = torque under range;
δ = torque over range

TABLE 19
Viscosities for Cream Formulation by Two Methods (in cPs)

Lot 2020-07-05
pH 5 0.6/Carbopol/No EDTA

| RPM | Brookfield | Cone & Plate |
|---|---|---|
| 0.3 | 938,300 | 288,300 |
| 0.5 | 581,000 | |
| 0.6 | 498,300 | 160,000 |
| 0.8 | 399,300 | |
| 1 | 326,500 | |
| 1.5 | 232,000 | 75,000 |
| 2 | 180,000 | |
| 2.5 | 144,000 | |
| 3 | 117,800 | 43,170 |
| 4 | 89,250 | |
| 5 | 73,200 | |
| 6 | 60,000 | 25,250 |
| 10 | 39,000 | |
| 12 | 34,000 | 15,130 |
| 20 | 22,970 | |
| 30 | | 7,933 |
| 60 | | 5,125 |

A summary of the properties of the tested compositions is shown in the table below:

TABLE 20
Summary of Composition Properties

| | | | Clotrimazole | | | | |
|---|---|---|---|---|---|---|---|
| | | | and | | Betamethasone | Betamethasone | Betamethasone |
| | | | Betamethasone | Clotrimazole | Dipropionate | Dipropionate | Dipropionate |
| | | | Dipropionate | Vaginal | Gel, | Cream, USP, | Cream, USP, |
| | | “pH 5 with | Cream, USP, | Cream, USP, | Augmented, | Augmented, | Augmented, |
| | “pH 5” | EDTA” | 1%/0.05% | 1% | 0.05% | 0.05% | 0.05% |
| Manufacturer | | | NorthStar | Sunmark | Taro | Taro | Perrigo |
| NDC # | | | 16714-496-01 | 49348-793-76 | 51672-1309-3 | 51672-1310-3 | 45802-376-32 |
| Lot # | 2019-11-3 | 2020-01-1 | 323547 | E14JA | E870131687 | L870534014 | 8LT0424 |
| pH | 5.0 | 5.0 | 6.2 | 5.1 | 3.8 | 4.6 | 3.3 |
| Tonicity | 301 | 306 | 1,582 | 153 | 9,735 | 1,456 | 1,308 |
| (mOsm/kg) | | | | | | | |
| Globule | | | | | | | |
| Size (μm) | | | | | | | |
| As Is | | | | | | | |
| Dn10 | 1.51 | 1.70 | 0.33 | 0.39 | Gel does | Could | Could |
| Dn50 | 2.84 | 2.67 | 0.78 | 1.14 | not have | not be | not be |
| Dn90 | 4.95 | 4.35 | 1.52 | 2.43 | globules | determined | determined |
| Number | 3.12 | 2.91 | 0.88 | 1.34 | | | |
| Mean | | | | | | | |
| Autoclave | | | | | | | |
| 110° C. | | | | | | | |
| 10 minutes | | | | | | | |
| Dn10 | 0.96 | 1.23 | | | | | |
| Dn50 | 1.46 | 1.79 | | | | | |
| Dn90 | 2.14 | 2.66 | | | | | |
| Number | 1.54 | 1.91 | | | | | |
| Mean | | | | | | | |
| Autoclave | | | | | | | |
| 130° C. | | | | | | | |
| 1 minutes | | | | | | | |
| Dn10 | | 1.26 | | | | | |
| Dn50 | | 1.71 | | | | | |
| Dn90 | | 2.43 | | | | | |
| Number | | 1.83 | | | | | |
| Mean | | | | | | | |
| Particle | | | | | | | |
| Size (μm) | | | | | | | |
| As Is | | | | | | | |
| Dn10 | 1.17 | 1.15 | 7.71 | 0.44 | None | None | None |
| Dn50 | 2.54 | 2.13 | 11.74 | 0.91 | Observed | Observed | Observed |
| Dn90 | 4.85 | 4.13 | 18.97 | 1.81 | | | |
| Number | 2.91 | 2.46 | 12.72 | 1.07 | | | |
| Mean | | | | | | | |
| Autoclave | | | | | | | |
| 110° C. | | | | | | | |
| 10 minutes | | | | | | | |
| Dn10 | 0.87 | 1.18 | | | | | |
| Dn50 | 1.54 | 1.80 | | | | | |
| Dn90 | 2.71 | 2.96 | | | | | |
| Number | 1.72 | 1.99 | | | | | |
| Mean | | | | | | | |
| Autoclave | | | | | | | |
| 130° C. | | | | | | | |
| 1 minutes | | | | | | | |
| Dn10 | | 1.25 | | | | | |
| Dn50 | | 1.71 | | | | | |
| Dn90 | | 2.55 | | | | | |
| Number | | 1.87 | | | | | |
| Mean | | | | | | | |
| Viscosity | | | | | | | |
| (cPs) | | | | | | | |
| 0.3 RPM | 938,300 | 638,300 | 275,000 | 275,000 | 1,158,000 | Out of | 788,300 |
| | | | | | | Range | |
| 0.5 RPM | 581,000 | 421,000 | 149,000 | 165,000 | 745,000 | Out of | 494,000 |
| | | | | | | Range | |
| 0.6 RPM | 498,300 | 365,800 | 124,100 | 148,300 | 633,300 | 92,500 | 411,600 |
| 1 RPM | 326,500 | 245,000 | 93,500 | 127,500 | 403,500 | 91,000 | 264,000 |

### Example 6: Ototoxicity Study in Guinea Pigs

Otoscopy-guided intratympanic (IT) injection was performed in guinea pigs followed by analysis of clearance of the test article (2020-01-01, as prepared in the foregoing examples, pH 5, including EDTA) from the middle ears.

Hearing was assessed at baseline in the left ear using auditory brainstem response (ABR) thresholds (4, 10, 20 kHz). 16 animals (8 male and 8 female) were administered 50 μL bilateral injections of the test article and 8 animals (4 male and 4 female) were administered 50 μL bilateral injections of saline. The 16 animals receiving the test article were randomized into 8 post-injection survival timepoints (days 1, 3, 5, 7, 10, 14, 21 and 28) at which time hearing was again assessed in the left ear using ABR thresholds (4, 10, 20 kHz). The control animals were allowed to survive for 1 or 28 day timepoints and likewise assessed using ABR thresholds. After each timepoint, the relevant animal(s) were sacrificed and a bilateral bullostomy was conducted to allow for examination of each middle ear and to document the presence of any cream and any edema or erythema.

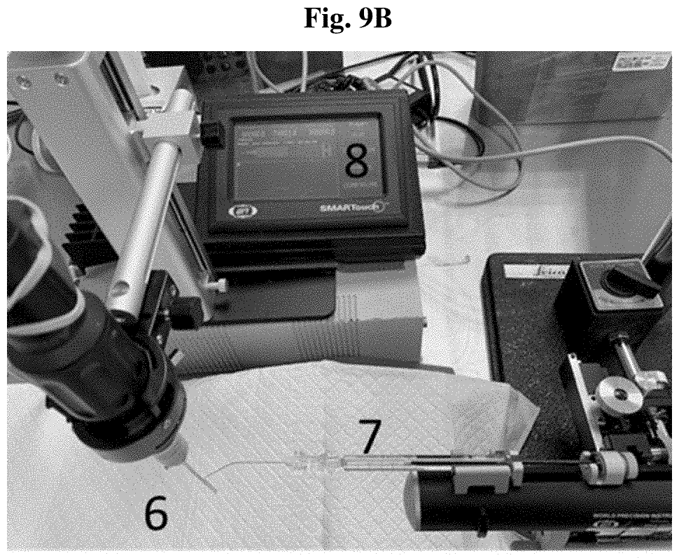

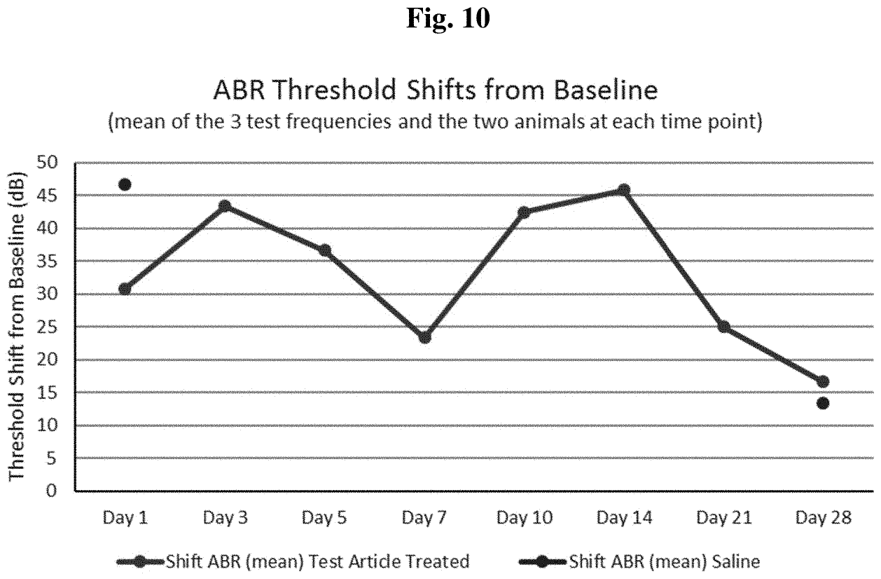

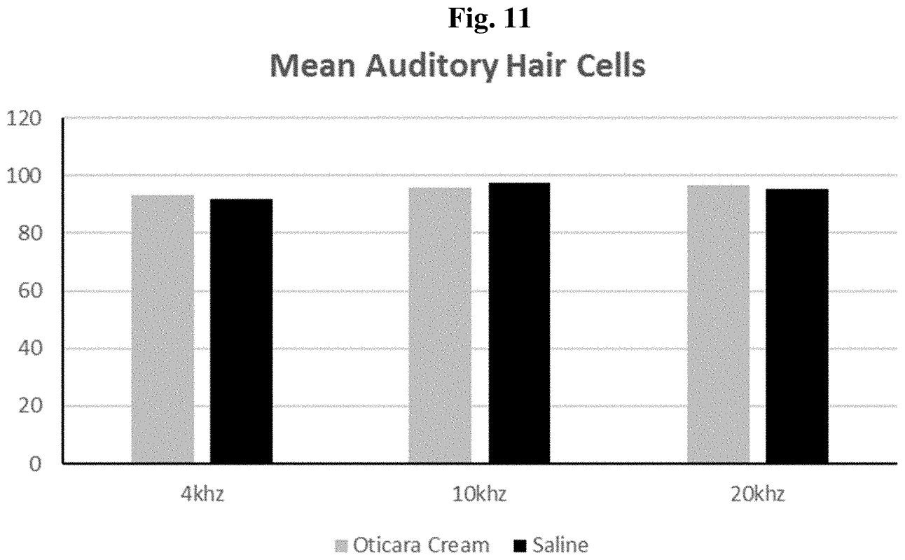

IT injections were performed with the aid of a 1.9 mm endoscope placed in the ear canal near the tympanic membrane (TM), allowing visualization and image capture of the TM before, during, and after the injections. Injections were performed using Becton Dickinson Exespine 0.5 mm×90 mm spinal needles, beveled to allow penetration of the TM but with a shortened shank to reduce the likelihood of damage to underlying structures. The non-beveled versus beveled tips are shown in FIG. 8 while the injection set-up is shown in FIGS. 9A-9B (6=stereotaxially placed microinjection syringe; 7=microinjection syringe; and 8=microinjection control system).

Injections delivered a precise volume of 50 μL of either the test article cream or saline over 10 seconds with the use of a World Precision Instruments Micro Injection System.

Bilateral otoscopy examinations were performed immediately before and after the injections and for up to 7 days, and again at necropsy.

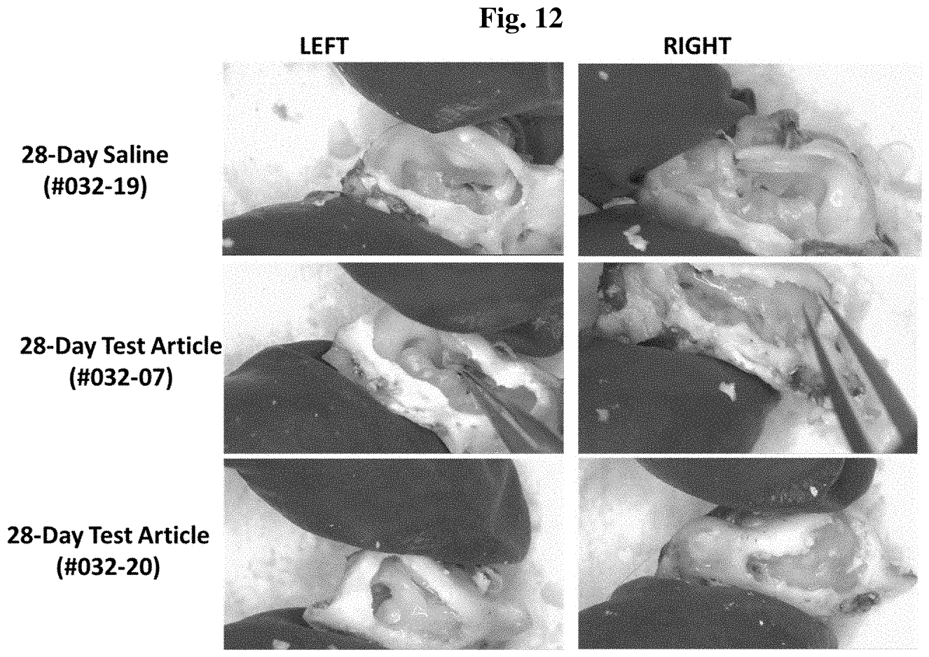

The ABR test results are provided in the table below, including the shift in ABR as thresholds. FIG. 10 shows the mean threshold shifts from baseline as a function of survival time and demonstrates that the thresholds returned to near-normal levels, similar to saline-treated ears.

TABLE 21
ABR Test Results

| Time | | | ABR Shift | | | |
|---|---|---|---|---|---|---|
| point | Animal # | Rx | 4 kHz | 10 kHz | 20 kHz | Mean |
| Day 1 | 10M | TA | 45 | 50 | 0 | 32 |
| Day 1 | 11F | TA | 10 | 45 | 35 | 30 |
| Day 1 | 8M | saline | 35 | −5 | 5 | 12 |
| Day 3 | 9M | TA | 65 | 25 | 20 | 37 |
| Day 3 | 16F | TA | 40 | 50 | 40 | 43 |
| Day 5 | 4M | TA | 0 | 35 | 5 | 13 |
| Day 5 | 14F | TA | 30 | 45 | 25 | 33 |
| Day 7 | 3M | TA | 30 | 35 | 35 | 33 |
| Day 7 | 18F | TA | 0 | 0 | 30 | 10 |
| Day 7 | 22M | Saline | 30 | 55 | 55 | 47 |
| Day 7 | 25F | Saline | 45 | 45 | 45 | 45 |
| Day 10 | 2M | TA | 35 | 35 | 20 | 30 |
| Day 10 | 17F | TA | 65 | 80 | 40 | 62 |
| Day 14 | 5M | TA | 25 | 35 | 45 | 35 |
| Day 14 | 15F | TA | 55 | 70 | 35 | 53 |
| Day 14 | 26F | Saline | 40 | 35 | 55 | 43 |
| Day 21 | 6M | TA | 30 | 5 | 35 | 23 |
| Day 21 | 13F | TA | 15 | 20 | 10 | 15 |
| Day 21 | 21M | Saline | −10 | −5 | −5 | −7 |
| Day 21 | 24F | Saline | 20 | 10 | 10 | 13 |
| Day 28 | 7M | TA | 15 | 15 | 40 | 23 |
| Day 28 | 20F | TA | 15 | 0 | 5 | 7 |
| Day 28 | 23M | Saline | −10 | −5 | 0 | −5 |
| Day 28 | 19F | Saline | 5 | 10 | 5 | 7 |

Cytocochleograms were conducted on 8 cochleae, a TA and saline treated cochlea at the time points of Days 1, 7, 21, and 28. The cochleae were qualitatively assessed for damage in the most extreme basal part of the cochlea and quantitatively assessed for the number of inner (IHC) and outer hair cells (OHC). Table 4 provides the raw IHC and OHC counts for each subject. IHC counts ranged from a total of 61-65 across all three frequency regions for saline treated animals and 61-62 for TA treated. Total OHC counts across all three frequency regions ranged from 211-224 in saline-treated controls and 220-227 in TA-treated samples. No evidence of frequency-specific loss of hair cells was found in the quantitative hair cell count assessments. In addition to the quantitative assessment at specific sound frequency locations, a qualitative assessment of the most extreme basal end of the cochlea was also conducted using lower magnification images. No qualitative evidence of hair cell loss in this extreme base region was apparent for any sample except #18F, which was treated with TA. However, this sample's most extreme base section also had major dissection damage that cut off all of the outer hair cells, leaving only inner hair cells to assess. Of the remaining inner hair cells, there appeared to be moderate hair cell loss. However, given the small sample size and the fact that this portion of cochlea was damaged from dissection, this qualitative observation might be artifactual.

Table 22 below provides the hair cell count results of the cytochleograms. These results demonstrate that the test article did not cause hair cell loss.

TABLE 22
Hair Cell Count Results

| | | | Inner Hair Cell Count | | | | Outer Hair Cell Count | | | |
|---|---|---|---|---|---|---|---|---|---|---|
| | | | 4 | 10 | 20 | | 4 | 10 | 20 | |
| Time point | ID # | Rx | kHz | kHz | kHz | Total | kHz | kHz | kHz | Total |
| Day 1 | 10M | TA | 20 | 21 | 21 | 62 | 74 | 75 | 78 | 227 |
| Day 1 | 8M | saline | 19 | 21 | 21 | 61 | 68 | 73 | 70 | 211 |
| Day 7 | 18F | TA | 21 | 19 | 22 | 62 | 70 | 78 | 79 | 227 |
| Day 7 | 22M | Saline | 18 | 24 | 22 | 64 | 72 | 82 | 76 | 230 |
| Day 21 | 13F | TA | 20 | 21 | 20 | 61 | 77 | 71 | 73 | 221 |
| Day 21 | 24F | Saline | 21 | 20 | 22 | 63 | 76 | 74 | 70 | 220 |
| Day 28 | 7M | TA | 19 | 22 | 20 | 61 | 71 | 76 | 73 | 220 |
| Day 28 | 23M | Saline | 20 | 22 | 23 | 65 | 73 | 74 | 77 | 224 |

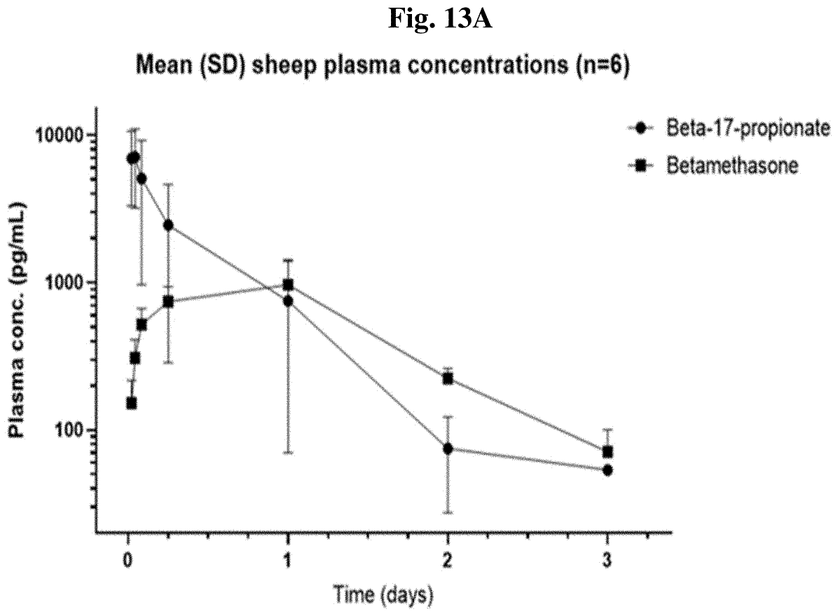

FIG. 11 depicts the mean auditory hair cell counts (per 200 μm) for each frequency range across all animals for test article (gray bars) and saline (black bars) groups.

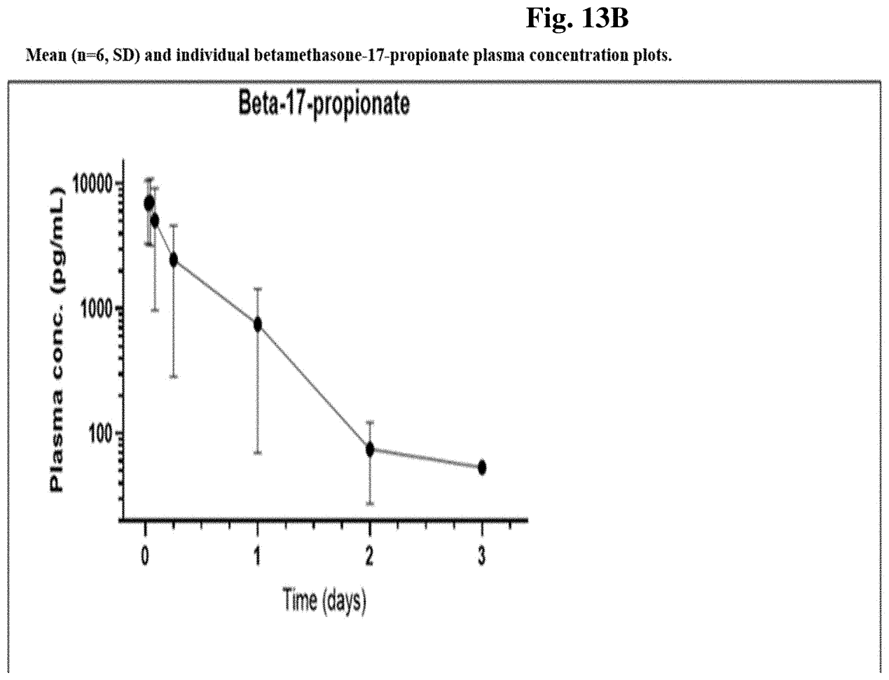

FIG. 12 depicts still images of the middle ear for the 28-day survival animals—1 with saline (top row); and 2 with the test article (middle and bottoms rows). As shown, both ears of the saline-treated animal appeared normal with no fluid visible. At day 28, one ear appeared normal (middle row, left column) with just a small amount of cream on the ossicles. The other 3 ears had a gel-like mass filling the majority of the middle-ear space (˜20-30% air space). This was similar to the findings for the 21-day animals where the gel-like mass included adhesions (to the TM, ossicles, cochlea and surrounding walls) that made it difficult to dissect away from surrounding tissues, although it appeared there was more air space in the gel-like mass at 28-days versus 21-days. The consistency of the gel-like mass was similar between the two timepoints. Minor erythema and inflammation of the canals after the treatment, which typically subsided by about 21-days post-treatment, was observed with the exception of the right ear for animal #032-07 which was swollen and did not allow visualization of the TM.

### Example 7: Microbial Testing of Cream with and without Clotrimazole/Betamethasone

Cream composition 2020-01-01 and placebo composition 2020-01-04 described in the foregoing examples were assessed by USP 51 for antimicrobial effectiveness. The results for each composition against the 5 microorganisms of the USP 51 test at full strength are shown in Tables 23-28 below.

TABLE 23
Effect of Compositions on Microbial Growth

| | Cream Composition | Placebo Composition |
|---|---|---|
| E. coli (8739) | 2020-01-01 | 2020-01-04 |
| Initial Count CFU/g | 1.4 × 10⁵ | 1.4 × 10⁵ |
| 6 Hour Count CFU/g | 1.5 × 10⁴ | 1.7 × 10⁴ |
| Log Reduction | 1.0 | 0.9 |
| 24 Hour Count CFU/g | 200 | 800 |
| Log Reduction | 2.8 | 2.2 |
| 7 Day Count CFU/g | <100 | <100 |
| Log Reduction | >3.1 | >3.1 |

TABLE 24
Effect of Compositions on Microbial Growth

| | Cream Composition | Placebo Composition |
|---|---|---|
| S. aureus (6538) | 2020-01-01 | 2020-01-04 |
| Initial Count CFU/g | 1.1 × 10⁵ | 1.1 × 10⁵ |
| 6 Hour Count CFU/g | 1.9 × 10⁴ | 2.4 × 10⁴ |
| Log Reduction | 0.8 | 0.7 |
| 24 Hour Count CFU/g | 4.2 × 10³ | 5.1 × 10³ |
| Log Reduction | 1.4 | 1.3 |
| 7 Day Count CFU/g | <100 | <100 |
| Log Reduction | >3.0 | >3.0 |

TABLE 25
Effect of Compositions on Microbial Growth

| | Cream Composition | Placebo Composition |
|---|---|---|
| P. aeruginosa (9027) | 2020-01-01 | 2020-01-04 |
| Initial Count CFU/g | 1.5 × 10⁵ | 1.5 × 10⁵ |
| 6 Hour Count CFU/g | <100 | <100 |
| Log Reduction | >3.2 | >3.2 |
| 24 Hour Count CFU/g | <100 | <100 |
| Log Reduction | >3.2 | >3.2 |
| 7 Day Count CFU/g | <100 | <100 |
| Log Reduction | >3.2 | >3.2 |

TABLE 26
Effect of Compositions on Microbial Growth

| | Cream Composition | Placebo Composition |
|---|---|---|
| C. albicans (10231) | 2020-01-01 | 2020-01-04 |
| Initial Count CFU/g | 2.0 × 10⁵ | 2.0 × 10⁵ |
| 6 Hour Count CFU/g | 7.9 × 10⁴ | 8.4 × 10⁴ |
| Log Reduction | 0.4 | 0.4 |
| 24 Hour Count CFU/g | 1.6 × 10⁴ | 1.4 × 10⁴ |
| Log Reduction | 1.1 | 1.2 |
| 7 Day Count CFU/g | <100 | <100 |
| Log Reduction | >3.3 | >3.3 |

TABLE 27
Effect of Compositions on Microbial Growth

| | Cream Composition | Placebo Composition |
|---|---|---|
| A. Brasiliensis (16404) | 2020-01-01 | 2020-01-04 |
| Initial Count CFU/g | 2.0 × 10⁵ | 2.0 × 10⁵ |
| 6 Hour Count CFU/g | 3.8 × 10⁵ | 4.5 × 10⁵ |
| Log Reduction | −0.3 | −0.4 |
| 24 Hour Count CFU/g | 3.0 × 10⁵ | 2.3 × 10⁵ |
| Log Reduction | −0.2 | −0.1 |
| 7 Day Count CFU/g | 4.1 × 10⁴ | 4.6 × 10⁴ |
| Log Reduction | 0.7 | 0.6 |

### Example 8: Human Clinical Study in Sinusitis Patients

A human clinical trial using a cream of the present disclosure to assess the safety and efficacy profile of the cream to treat sinusitis will be performed. The investigational drug product will be a betamethasone dipropionate cream (0.05%, 0.5 mg/g) that contains 0.9% benzyl alcohol, polysorbate 80, glycerin, EDTA disodium, Carbopol 980, polyoxyl 40 stearate, cetyl alcohol, glyceryl monostearate, petrolatum, Span 20, sodium hydroxide, and water (the same formulation as Table 28 with the exception that glycerin was used at 1.65% (w/w)). The cream will be applied using a 4-inch flexible tip applicator attached to a syringe that has been prefilled with the cream which is applied with the aid of an endoscope.

The cream will be stored at controlled room temperature.

A number, planned to be 50, patient post-FESS, aged 18 to 80 years and diagnosed with sinusitis and uncontrolled symptoms ongoing at least 30 days will be enrolled. All patients will have had previous bilateral ethmoidectomy and maxillary antrostomy. A single dose of 5 cc of cream will be placed onto the left and right inflamed sinus mucosa (total of 10 cc). This corresponds to 1.288 mg of betamethasone dipropionate per side or a total of 2.576 mg of betamethasone dipropionate. Follow-up will be at days 5 and 21 post-application for evaluation and safety assessment. Patients will undergo a run-in period of 7 days between screening and the application of the cream to measure disease state prior to treatment.

Inclusion Criteria Include:
1. Male or non-pregnant, non-lactating female, 18 to 80 years of age.
2. Bilateral ethmoidectomy and maxillary anostromy within the past 20 years, but no less than 6 months prior.
3. Clinical diagnosis of exacerbation of sinusitis with the current episode of uncontrolled symptoms lasting at least 30 days. Both ethmoids and maxillaries will be treated.
4. A score ≥2 on at least two of the “Cardinal” symptoms on a scale of 0 to 3.
5. A score ≥2 on the “Cardinal” symptom Obstruction and Congestion scale of 0 to 3.
6. Must present with mucosal edema.
7. No more than a mild polyp burden that will not interfere with placement of cream per physician assessment.
8. At least 1 trial of topical corticoid sprays or irrigations for a minimum of 1 month prior to screening.
9. Able to understand and to provide signed informed consent.
10. Females of childbearing potential must have a negative urine pregnancy test at screening and agree to use an acceptable birth control methods.
11. Agree to refrain from water immersion of the sinuses during the conduct of the study.
12. Agree to refrain from chronic use of ocular steroidal or non-steroidal anti-inflammatory drug, and biologics for asthma or sinusitis from screening to the exit visit. Antiallergic medications are only allowed if patients would continue their antiallergy medication at a consistent dose from screening to the exit visit.
13. Patients who take analgesics or other non-steroid-containing maintenance medications (e.g. for arthritis) will be allowed in the study provided that the dose have been stable for at least 8 weeks prior to enrollment and must remain stable during the course of the study.
14. Normally active and otherwise judged (in the opinion of the Investigator) to be in generally good health on the basis of medical history and physical examination.
15. Patients and/or caregiver(s) who are able to adhere to the visit schedule and protocol requirements and available to complete the entire study.

Exclusion Criteria Include:
1. Females who are pregnant, breastfeeding, or who wish to become pregnant during the study period.
2. Signs and symptoms of current episode of sinusitis of less than 30 days.
3. Asthma that is uncontrolled.
4. History of diabetes mellitus, immune deficiency, allergy or intolerance to corticosteroids, oral steroid-dependent condition, clinical evidence of acute bacterial sinusitis, or clinical evidence of invasive fungal sinusitis.
5. History or diagnosis of glaucoma or ocular hypertension, presence of cataracts grade+3 or higher, or presence of posterior subscapular cataract.
6. Clinically diagnosed sino-nasal disease other than exacerbation of sinusitis (e.g., congenital abnormalities of the sino-nasal area, obstructive bony exostosis or tumors, upper respiratory infections, including varicella and herpes simplex infection, cellulitis).
7. Known or suspected hypersensitivity to betamethasone dipropionate or topical anesthesia.
8. Local sinus abnormalities such as abscess, nasal septal perforation, or severe nasal blockage by nasal polyps that prevented access to or visualization of the affected sinus.
9. Unwilling to discontinue use of nasal medications, rinse or spray for 5 days after treatment.
10. Prior sinus surgery within 3 months of study entry in the affected sinus.
11. Patient with any type of device (e.g., PROPEL) in the nose or sinus.
12. Surgical procedures in the nose or sinus after application of the cream for the duration of the study, unless prescribed by the Investigator after exiting the patient from the study.
13. Previous enrollment in this study.
14. Systemic or topical immunosuppressive drugs or immunomodulators (e.g., azathioprine, infliximab, calcineurin inhibitors).
15. Any significant mental or mental/psychiatric condition(s) which, in the investigator's judgment, would interfere with the ability to provide an informed consent or comply with study instructions, or that might confound the interpretation of the study results or put the patient at undue risk. This should include: recent history of (within the past 12 months) or strong potential for, alcohol or substance abuse.
16. Non-study medications such as acetaminophen or ibuprofen may be used for pain. All such use should be reported to the study staff.

Safety assessments will include documenting AEs which will be collected for the duration of the study (through exit visit for each patient). This reporting group will include all participants who received study drug. AEs will be obtained as solicited comments from the study patients and/or caregivers or observations by the study investigator. This will also include reporting serious adverse events (SAEs).

At screening and each clinic visit an ENT (head and neck) inspection will be conducted.

Six patients will be enrolled into a PK study. Plasma drug concentration will be measured at pretreatment, then 24 hours following dosing, or at times to be determined.

Patients will need to start the study between 8 and 9 am to test morning serum cortisol and/or ACTH levels. Cortisol level will be measured: before dosing at the treatment visit, then day 5 and at the exit visit, day 21 post-dosing.

Intraocular pressures (IOPs) will be measured at screening and at the exit visit. IOPs are required to be normal, 12-22 mm Hg, for enrollment into the study.

Cream retention will be measured daily for PK patients until no longer visible and for all patients at days 5 and 21. Presence or absence of cream in the sinus will be assessed via endoscopic examination.

The 4-Cardinal Symptoms Score Daily Diary will be completed daily by the patient during the 7-day run-in period until the exit visit. The “Cardinal” symptoms are Obstruction and Congestion, Facial Pail and Pressure, Nasal Discharge and Loss of Sense of Smell. These symptoms are reported daily by the patient as 0—None, 1—Mild, 2—Moderate and 3—Severe. Change in the 7 day total average prior to treatment and 7 day total average prior to exit will be the primary measure of efficacy. An exploratory measure of efficacy will be change in a visual analogue scale (VAS) for common sino-nasal symptoms completed by the patient pre-treatment and at the exit visit (“Cardinal” symptoms or Doulaptsi, et Al. Visual analogue scale for sino-nasal symptoms severity correlates with sino-nasal outcome test 22: paving the way fora simple outcome tool of CRS burden. Clin Transl Allergy, 2018; 8: 32)f. An additional exploratory measure of efficacy will be change in Modified Lund Mackay Endoscopy Scores (pre-treatment verse day 21) based on video assessments by three independent, blinded physicians (Snidvongs, et Al. Modified Lund Mackay Postoperative Endoscopy Score for defining inflammatory burden in chronic rhinosinusitis. Rhinology, 52: 53-59, 2013).

Via use of a nasal endoscope, the amount of cream present in the sinus will be rated on the following scale: Visible (any amount) and Not Visible. This will be measured daily for the first six patients and at the study visit day 5 and the exit visit for all patients.

### Example 9: Phase 2 Clinical Trial in Patients with Confirmed or Suspected Otomycosis

A multicenter, sham-controlled, double-blind, prospective, randomized phase 2 clinical study of a single dose of clotrimazole (1%)/betamethasone (0.025%) combination cream, clotrimazole (1%) cream, betamethasone (0.025%) cream or sham (air injection) will be used to treat patients with confirmed or suspected otomycosis.

The patients will be grouped into four treatment groups: Group 1 will receive the clotrimazole/betamethasone cream at or below an established maximum potential dose per treated ear of 15 mg clotrimazole and 0.375 mg betamethasone; Group 2 will receive the clotrimazole cream at or below an established maximum potential dose per treated ear of 15 mg clotrimazole; Group 3 will receive the betamethasone cream at or below an established maximum potential dose per treated ear of 0.375 mg betamethasone; Group 4 will receive the sham (air) treatment. Each of the creams will be formulated as described in the presented disclosure.

Groups 1-3 will have their external auditory canal (EAC) cleaned, if necessary, and receive one application of the cream to fill the EAC. Group 4 will have their EAC cleaned, if necessary, and receive application of air in the EAC. Study participants will return on day 10+/−1 following treatment for evaluation. Primary efficacy will be assessed based on resolution of signs and symptoms on day 10+/−1 post-application as judged by a blinded assessor for complete resolution of erythema, edema, otorrhea, and tenderness to compare the clotrimazole/betamethasone cream to the sham (air) treatment. Secondary objectives to be assessed include:
1. Resolution of signs and symptoms on day 10+/−2 following treatment as judged by a blinded assessor for complete resolution of erythema, edema, otorrhea, and tenderness to compare the clotrimazole/betamethasone cream to the clotrimazolecream.
2. Resolution of signs and symptoms on day 10+/−2 following treatment as judged by a blinded assessor for complete resolution of erythema, edema, otorrhea, and tenderness to compare the clotrimazole/betamethasone cream to the betamethasone cream.
3. Time to resolution of itch as reported by the patient via a daily diary to compare the clotrimazole/betamethasone cream to the sham (air) treatment.
4. Time to resolution of itch as reported by the patient via a daily diary to compare the clotrimazole/betamethasone cream to the clotrimazole cream.
5. Time to resolution of itch as reported by the patient via a daily diary to compare the clotrimazole/betamethasone cream to the betamethasone cream.
6. Time to resolution of pain as reported by the patient via a daily diary to compare the clotrimazole/betamethasone cream to the sham (air) treatment.
7. Time to resolution of pain as reported by the patient via a daily diary to compare the clotrimazole/betamethasone cream to the clotrimazole cream.
8. Time to resolution of pain as reported by the patient via a daily diary to compare the clotrimazole/betamethasone cream to the betamethasone cream.
9. Clinical cure defined as no further treatment is required on Test of Cure (TOC)visit, as judged by a blinded assessor for erythema, edema, otorrhea, and tenderness, and review of the symptoms with the patient to compare the clotrimazole/betamethasone cream to the sham (air) treatment.
10. Clinical cure defined as no further treatment is required on Test of Cure (TOC)visit, as judged by a blinded assessor for erythema, edema, otorrhea, and tenderness, and review of the symptoms with the patient to compare the clotrimazole/betamethasone cream to the clotrimazole cream.
11. Clinical cure defined as no further treatment is required on Test of Cure (TOC)visit, as judged by a blinded assessor for erythema, edema, otorrhea, and tenderness, and review of the symptoms with the patient to compare the clotrimazole/betamethasone cream to the betamethasone cream.
12. Fungal eradication to compare the clotrimazole/betamethasone cream to the sham(air) treatment.
13. Fungal eradication to compare the clotrimazole/betamethasone cream to the clotrimazole cream.
14. Fungal eradication to compare the clotrimazole/betamethasone cream to the betamethasone cream.
15. Bacterial eradication to compare the clotrimazole/betamethasone cream to the sham (air) treatment.
16. Bacterial eradication to compare the clotrimazole/betamethasone cream to the clotrimazole cream.
17. Bacterial eradication to compare the clotrimazole/betamethasone cream to the betamethasone cream.
18. Safety based on reported adverse events.

260 patients will be enrolled in the study and will be 8 years old or older and have been diagnosed with otomycosis (suspected or confirmed) and will meet all the inclusion/exclusion criteria. Patients will return for evaluation of the effectiveness of their treatment at day 10+/−2 (Time of Cure (TOC)). Patients or caregiver(s) will record discomfort from pain and itch in treated ear(s), in a daily diary at home according to a scale for pain of from: no pain, mild pain, moderate pain, severe pain, extreme pain to pain as bad as could be; and according to a scale for itch from: no itch, mild itch, moderate itch to severe itch. The time of cessation of pain and itch will be defined as the first time point that pain and itch is absent (morning or evening) and does not recur in any subsequent diary entries.

Inclusion criteria for the study will include:
1. Male or non-pregnant, non-lactating female aged at least 8 years.
2. Clinical diagnosis of unilateral or bilateral otomycosis (suspected or confirmed, could also be polymicrobial including fungi/yeast).
3. Combined numeric severity score of ≥4 in at least 1 affected ear at the screening visit for tenderness, discharge, erythema, and edema. Each measure is scored as follows: 0=none [complete absence of any signs or symptoms], 1=mild [slight/detectable], 2=moderate [definitely present], 3=severe [marked, intense]. In patients with bilateral otomycosis, only one ear must meet this criterion, and both ears are assessed, cultured, and treated identically as per group randomization.
4. Females of childbearing potential at screening agree to use acceptable birth control methods.
5. Agree to refrain from water immersion of the ears during the conduct of the study.
6. Patients who take analgesics or other non-steroidal-containing maintenance medications (e.g., for arthritis) will be allowed in the study provided that the dose has been stable for at least 8 weeks prior to enrollment and must remain stable during the course of the study
7. Able to understand and provide signed informed consent. The parent or legal guardian of a patient under the age of 18 must also have read and signed the written informed consent to participate prior to study participation.
8. Normally active and otherwise judged in the opinion of the Investigator to be in good health on the basis of medical history and physical examination.
9. Patients and/or caregiver(s) who are able to adhere to the visit schedule and protocol requirements and available to complete the entire study

Exclusion criteria will include:
1. Current diagnosis of malignant otitis externa.
2. Known or suspected hypersensitivity to or allergy to clotrimazole, betamethasone dipropionate, or any other component of the study medications.
3. Ear canal abscess.
4. Diagnosis of diabetes mellitus of any type.
5. Patient who uses ear plugs, headphones or earbuds and is unwilling to discontinue their use during the study period.
6. Prior otologic surgery within 1 year of study entry in the affected ear.
7. Inability to discontinue use of systemic antibacterial medication prior to study treatment.
8. Current or previous use (within 3 days) of topical vinegar, alcohol or other astringents in external auditory canal of the affected ear(s).
9. Use of any systemic glucocorticoids.
10. Inability to discontinue use of hearing device or otic wick for the term of the study (screening to TOC visit) in treated ear(s).
11. Previous enrollment in this study.
12. Any significant medical or mental/psychiatric condition(s) which, in the PI's judgment, would interfere with the ability to provide informed consent or comply with study instructions, or might confound the interpretation of the study results or put the patient at undue risk.
13. Current enrollment in an investigational drug or device study or participation in such a study within 30 days of entry into this study.
14. Any reason the Principal Investigator believes the patient should not participate

During each study visit, a score will be recorded for each of the following signs and systems using the scoring system described above. Signs: tenderness of the tragus and pinna, edema, discharge, and erythema. Symptoms: itch and pain, as reported by the patient.

At the first study visit, the patient's medical history will be obtained, including past ontological history (e.g. tinnitus, mastoidectomy, hearing loss, recurrent otitis externa, past tympanostomy). The date of onset of signs and symptoms related to otomycosis will be recorded as well as any concomitant medication. A head and neck examination will also be conducted. Clinical assessment of the affected ear(s) will also be performed as described above for the signs and symptoms. A culture specimen will also be collected from the EAC wall. Mechanical cleansing of the EAC, if necessary, will be performed. Treatment will be applied according to the group into which the patient is placed.

Patients or caregiver(s) will record itch and pain severity twice daily and any analgesic used for each pain. Any adverse events will also be recorded.

Patients will be clinically evaluated at day 10+/−2 post-treatment (TOC). Residual study drug will be removed if present. A blinded medical professional will examine the patient and record signs according to the 4-point scale described above. Itch and pain will be assessed based on patient reporting. A culture specimen will be collected from the EAC wall.

### Example 10: Local Absorption and Tolerance Study

A composition of the present disclosure containing 0.05% (w/w) betamethasone dipropionate will be tested in a sheep model to assess local absorption and tolerance.

6 Merino sheep will undergo bilateral frontal trephination (placement of small metal cannulae through a drill hole into both frontal sinuses) under general anesthesia. Sheep will be randomized to receive the test formulation in one sinus and saline control in the contralateral sinus with randomization of the treated sinuses. The test formula will be administered to fill the whole frontal sinus until the cream appears in the nasal cavity. The volume administered will be measured. The trephines will be removed and the skin closed over the drill holes.

Sheep will recover in pens and be monitored for general wellbeing. Nasal discharge will be recorded twice daily.

Blood will be collected from the sheep, for example, pre-dose, 1, 2, 6, 24, 48, and 72 hours post-dose for pharmacodynamics and pharmacokinetic analysis. Certain of these and, optionally, additional timepoint blood samples will be collected to measure ACTH and/or cortisol levels.

Sheep will be euthanized 10 days after dosing. Sinus tissues will be assessed by a blinded veterinary pathologist for macroscopic evaluation and histopathology. For macroscopic evaluation, gross evaluation of mucosal integrity and mucosal irritation will be performed qualitatively according to a scale and photos will be taken. Remnants of any cream will be observed and assessed in a qualitative way and photos will be taken. For histopathology, scanning electron microscopy will be performed to evaluate ciliary and tight junction morphology of sinus mucosa. Paraffin-embedded histopathology will also be performed by haematoxylin and eosin staining. The epithelial layer will be evaluated for integrity and signs of metaplasia. Mucosa will be evaluation for inflammation and fibrosis.

### Example 11: Membrane Diffusion and Permeability Testing

The diffusion and retention properties of compositions of the present disclosure will be tested on cadaver skin and mucosa. The composition will varying amounts of betamethasone dipropionate. Drug permeation through cadaver skin and excised nasal mucosa was measured by HPLC. Both the composition with the active agents and the composition without active agents will be tested.

Permeation across the skin will be measured using surgically excised fresh human skin in Franz diffusion cells. The effect of doses applied was also be assessed (dose=0.2 g, 0.5 g, and 1 g, n=6 and n=3 for controls). Samples were drawn from the receptor fluid at 0.5, 1, 2, 4, 6, 8, 12, 24 and 48 hours. HPLC analysis was performed to measure the drugs as described in Example 4. Statistical analysis was be performed using a Wilcoxon Rank-Sum test for non-normally distributed data (α=0.05).

Permeation across the nasal mucosa was measured across excised fresh bovine nasal mucosa in Franz diffusion cells. Doses up to 1 g were tested. Samples were drawn from the receptor fluid at 0.5, 1, 2, 4, and 6 hours and, optionally, at 8, 12, 24 and 48 hours. HPLC analysis was be performed to measure the drugs as described in Example 4. Statistical analysis was performed using a Wilcoxon Rank-Sum test for non-normally distributed data (α=0.05).

% Permeation was found to be below the limit of quantification (<45 ng/mL) at all timepoints of 0, 0.5, 1, 2, 4, and 6 hours for bovine nasal mucosa (n/a for timepoints at 8, 12, 24 and 48 hours) and below the limit of quantification for all timepoints of 0, 0.5, 1, 2, 4, 6, 8, 12, 24 and 48 hours for human skin. This indicates a local effect of the cream composition.

### Example 12: Formulation Manufacturing

An alternative formulation of the betamethasone dipropionate cream was prepared having the following composition as shown in Table 28.

TABLE 28
Betamethasone Dipropionate Cream Formulation

| | |
|---|---|
| Material | % w/w |

Water Phase

| | |
|---|---|
| Betamethasone dipropionate | 0.0322% |
| Super Refined Polysorbate 80 | 5.0000% |
| Glycerin | 1.4500% |
| Disodium EDTA, Dihydrate | 0.0500% |
| Carbomer 980 | 0.6000% |
| Purified Water, USP | 78.4356% |

Oil Phase

| | |
|---|---|
| Betamethasone dipropionate | 0.0322% |
| Polyoxyl 40 Stearate | 1.0000% |
| Cetyl Alcohol | 1.0000% |
| Glyceryl Monostearate (GMS) | 0.5000% |
| Petrolatum | 8.0000% |
| Super Refined Span 20 | 3.0000% |

Combined Phase

| | |
|---|---|
| Super Refined Benzyl Alcohol | 0.9000% |

pH Adjustment

| | |
|---|---|
| Sodium Hydroxide Pellets | N/A |
| Total: | 100.0% |

The above formulation was scaled up from formulation development to manufacturing scale (2000 g).

### Preparation of the Water Phase

The lab scale process reported to begin with approximately 125-150 g of water dispensed to a 400 mL beaker. Using an overhead mixer with a 4 blade propeller, mixing of the water began at 200-300 rpm. EDTA and Glycerin were added to dissolve, and mixer speed was increased to ˜800 rpm for ˜1 minute. Mixer was turned off and Carbopol was slowly added by sprinkling on the surface and pulsing the mixer 2-5 times between each small addition to wet the material. Once all Carbopol was added, mixing resumed at 800-1000 rpm for 30 minutes, rotating the beaker every 5-10 minutes. Once thoroughly mixed, the pH was tested and adjusted using a 1% NaOH solution as needed to reach a target of pH 6. The mixture was QS with water, and mixed for ˜30 minutes. Mixer was then set to 200-300 rpm and Polysorbate 80 was carefully added to avoid foaming. Mixing continued ˜45 minutes with the container moved up and down (“milk shake” mixed) every 5-10 minutes to ensure even mixing throughout.

To avoid having to add an excess of 1% sodium hydroxide solution for pH adjustment which may risk over diluting the product, a 2% sodium hydroxide solution is proposed. Mixing times and speeds can be adjusted as needed using visual observations to determine dispersion while minimizing air entrapment. Suggested scale up parameters are provided in Table 29 below.

TABLE 29
Water Phase (Phase A) Parameter Comparison

| | | |
|---|---|---|
| | Formulation | Engineering and |
| Parameter | Development | cGMP Batch |

Water Phase

| | | |
|---|---|---|
| Container | 400 mL beaker | ~6 Qt. Stock Pot |
| Mixer/Blade | Overhead/4 | Overhead/4 |
| | Blade Propeller | Blade Propeller |

Initial Charge

| | | |
|---|---|---|
| Mixing Speed (RPM) | 800-1000 | TBD |
| Mixing Time (min) | >31 min | TBD |
| Order of Addition | Purified Water | Purified Water |
| | EDTA, Glycerin | EDTA, Glycerin |
| | Carbopol | Carbopol |

pH Adjustment/QS

| | | |
|---|---|---|
| NaOH Solution (%) | 1.0 | 2.0 |
| Mixing Time pH | 30 | NLT 5 |
| adjustment (min) | | |

Addition of Polysorbate 80

| | | |
|---|---|---|
| Mixer Speed (RPM) | 200-300 | TBD |
| Mixing Time (min) | ~45 | NLT 5 |

### Preparation of the Oil Phase

The lab scale process for the oil phase activities can be performed in-tandem with the water phase activities. Span 20, Petrolatum, Polyoxyl 40 Stearate, Cetyl Alcohol, and Glyceryl Monostearate (GMS) were added to a 250 mL beaker (ordered from liquid to most solid) equipped with a stir bar. The beaker was heated on a hotplate until ingredients reached a temperature of 65+/−5° C. During heating, materials were mixed with the stir bar at 100-350 rpm for ˜15 minutes, until most solids were melted. Mixing speed was reduced to 50-100 rpm, and ingredients mixed for ˜10 minutes until homogeneous.

In order to achieve a more robust and reproducible process at the larger 2000 g scale, it is proposed that the batches will utilize a hot water bath and overhead mixing for the oil phase. This should better simulate large scale jacketed tanks for future scalability. In addition, the mixing is proposed to be a propeller type impeller instead of a simple stir bar. Temperature, mixing speed and mixing time will be adjusted and recorded as needed in-process based on visual observations. Suggested scale up parameters are provided in Table 30 below.

TABLE 30
Oil Phase (Phase B) Parameter Comparison

| | | | |
|---|---|---|---|
| | Formulation | Engineering | |
| Parameters | Development | Batch | cGMP Batch |

Oil Phase

| | | | |
|---|---|---|---|
| Container | 250 ml | ~4 Qt. | ~4 Qt. |
| | beaker | Stock Pot | Stock Pot |
| Mixer/Blade | Stir Bar | Overhead/4 | Overhead/TBD |
| | | Blade | |
| | | Propeller | |

| | | |
|---|---|---|
| Order of Addition | Liquid to | Span 20 |
| | most solid | Petrolatum |
| | | Polyoxyl 40 Stearate |
| | | Cetyl Alcohol |
| | | Glyceryl Monostearate |

Initial Heating/Mixing

| | | | |
|---|---|---|---|
| Mixing Speed (RPMD) | 100-350 | TBD | TBD |
| Mixing Time (min) | ~15 | NLT 5 | NLT 5 |
| Target Temperature (° C.) | 65 ± 5 | 65 ± 5 | 65 ± 5 |

Reduced Mixing

| | | | |
|---|---|---|---|
| Mixing Speed (RPM) | 50-100 | N/A | N/A |
| Mixing Time (min) | ~10 | N/A | N/A |

API Addition

The lab scale process reported to change the mixing blade in the water phase to a higher shear disk impeller blade and mix for ˜5 minutes at the lowest rpm setting available. Water Phase was heated to a target of 62+/−3° C. and mixing speed was set to the highest rpm that does not induce foaming (˜1200+ rpm). Heating reported to take ˜30 minutes at the small lab-scale to reach target temperature. Betamethasone Dipropionate was dispensed appropriately, with half of the total amount being allocated to the Water Phase, and half to the Oil Phase. Water Phase mixer was turned off and Betamethasone Dipropionate was added to both phases. Mixing resumed, and both phases were mixed for 10-15 minutes while still being heated.

The following calculation was used to determine the approximate tip speed of the average lab-scale batch produced:

$$\frac{Blade Diameter ( inches )}{12 in / ft} \times π \times Mix Speed ( RPM ) = Tip Speed ( ft / min or FPM )$$

Based on the calculated average tip speed used during the lab scale batches (300 g and 600 g batch sizes), and based on the proposed mixer size for the engineering and cGMP scale, the approximate mixing speed at this larger scale is ˜760 RPM. However, the main indicator of what mixing speed to use will be visual cues to reduce the possibility of over incorporating air into the mixture.

Because the API is a highly potent compound for the inhalation route and must be contained in powder form, for the engineering and cGMP batches containment will be used in the dispensing process and surrounding the heated vessels while adding the active to the two phases. Suggested scale up parameters are provided in Table 31 below.

TABLE 31
Active Addition (Phase C) Parameter Comparison

| | | |
|---|---|---|
| | Formulation | Engineering and |
| Parameters | Development | cGMP Batches |

API Addition
Water Phase

| | | |
|---|---|---|
| Mixer/Blade | Overhead with | Overhead with 3″ |
| | 4.5 cm/5.0 cm | High Flow High |
| | Disk | Shear Disperser |
| Mixing Speed (RPM) | ~1200+ | 760* |
| Estimated Tip Speed (FPMD) | ~598 FPM | ~598 FPM |
| Target Temperature (° C.) | 62 ± 3 | 62 ± 3 |
| Mixing After Addition (min) | 10-15 | NLT 15 |

Oil Phase

| | | |
|---|---|---|
| Mixing Speed (RPM) | 50-100 | TBD |
| Mixing After Addition (min) | 10-15 | NLT 15 |

Combining of Phases

For the lab scale batch, mixing of the Water Phase was increased to ˜1800 rpm. As the Water Phase was mixing and still being heated, the hot Oil Phase was added into the Water Phase in 2-3 portions. The now combined emulsion was then removed from the heat and mixed ˜45 minutes, moving the container up and down in the mixture every 5-10 minutes to ensure homogeneous mixture. Benzyl Alcohol (Phase D) was added to the mixture after cooling to <30° C. and was mixed under high shear of ˜1800 rpm followed by ˜30 minutes at ˜1200 rpm moving the container up and down every 5-10 minutes. The lab scale emulsion was then QS with water reportedly based on beaker tare, theoretical mass, and Oil Phase loss, and then mixed for ˜10 minutes.

In order to achieve a more robust and reproducible process at the larger 2000 g scale, it is proposed that the batches will utilize a mixing shaft consisting of either 1 or 2 disk blades at various points up the shaft. Using the high flow high shear disperser blade may only require 1 blade to achieve sufficient mixing, but this will be evaluated in process. This should alleviate the need to move the container up and down in the so-called “milk shake” mix performed at the client's formulation development site, which poses a safety risk at the larger scale.

Based on the calculated average tip speed used during the lab scale batches, and based on the proposed mixer size for the larger scale, the approximate mixing speed at this larger scale will be ˜1120 rpm. However, the main indicator of what mixing speed to use will be visual cues, to reduce the possibility of over incorporating air into the mixture.

It is unclear what adjustment formula was used to adjust the final amount of water. QCL proposes to adjust the final water amount based on amount of API lost in the oil phase; however, if excessive losses occur there is a risk to sub-potency of the batch and the proportion of oil to water phases would not be consistent using this method. As an alternative, an overage of oil phase could be prepared and carefully portioned into the water phase to prevent having to make calculated adjustments from losses. It is unclear at this time how much overage would be required at this scale.

No additional pH measurements or adjustments were recorded in the lab scale process prior to QS of the final product. It is recommended that a pH check be incorporated and adjustments made as needed with the same 2% sodium hydroxide solution used in the water phase. Mixing times and speeds will be adjusted as needed using visual observations to determine dispersion while minimizing air entrapment. Suggested scale up parameters are provided in Table 32 below.

TABLE 32
Active Addition (Phase C) Parameter Comparison

| | | |
|---|---|---|
| | Formulation | Engineering and |
| Parameters | Development | cGMP Batches |

Combining of Phases

| Mixer/Blade | Overhead with | | Overhead with 3″ | |
|---|---|---|---|---|
| | 4.5 cm/5.0 cm | | High Flow High | |
| | Disk | | Shear Disperser | |
| Initial Mixing Speed (RPM) | ~1800+ | | 1120* | |
| Estimated Tip Speed (FPM) | ~881 | FPM | ~881 | FPM |
| Mixing Time (min) | ~45 | | NLTS | |
| Target Cooled | 30° | C. | <30° | C. |
| Temperature (° C.) | | | | |

Benzyl Alcohol Addition

| Mixing Speed (RPM) | *1800; | ~1200 | TBD | |
|---|---|---|---|---|
| Estimated Tip Speed (FPM) | ~881 | FPM | ~881 | FPM |

| | | | |
|---|---|---|---|
| Mixing After Addition (min) | Unknown; | ~30 | NLT 5 |

Water QS

| | | |
|---|---|---|
| Adjusted based on loss | Yes | TBD |
| Mix After QS (min) | ~10 | NLT 5 |

Below is an exemplary manufacturing procedure:

Water Phase
1. Obtain two (2) stock pots, one that will fit inside the other to be able to create a water bath for heating.
2. Add, in order, ˜½ of the USP Purified Water, Glycerin and Disodium EDTA.
3. Mix using an overhead mixer for NLT 5 minutes until EDTA is dissolved.
4. Stop mixer and slowly add Carbomer while periodically “pulsing” the mixer during addition. Mix for NLT 5 minutes until fully dispersed.
5. Measure pH and adjust to a target of 6.0 using 2% sodium hydroxide solution, as needed, mixing between measurements for at least 5 minutes.
6. Slowly add Polysorbate 80, mixing to disperse while preventing foaming.
7. Begin heating water bath to bring product temperature to 62+/−3° C.

Oil Phase
1. Obtain two (2) stock pots, one that will fit inside the other to be able to create a water bath for heating.
2. Add, in order, Span 20, Petrolatum, Cetyl Alcohol, Polyoxyl 40 Stearate, and GMS.
3. Begin heating water bath to melt the mixture and bring product temperature to 65+/−3° C.
4. Begin mixing using an overhead mixer until fully melted and combined.

API Addition and Phase Combination
1. Add appropriate amounts of Betamethasone Dipropionate to both the Water Phase and Oil Phase mixtures and mix while heating for NLT 15 minutes.
2. Stop mixing the Oil Phase and carefully remove the pot from heating in order to add it into the hot Water Phase.
3. Allow Oil Phase to fully incorporate into the Water Phase, then remove Combined Phase from heat while continuing to mix.
4. Allow Combined Phase to cool to a target of <30° C.
5. Measure pH and adjust to a target of 6 as needed.
6. Add Benzyl Alcohol while mixing. Mix for NLT 5 minutes.
7. Stop mixing, weigh container, and QS using USP Purified Water to adjusted target weight (adjusted for oil phase loss).

Packaging
1. Calculate density of the finished product and fill 10 cc syringes by weight to a target weight, equivalent to 5 mL.
2. Label syringes and package two (2) syringes in each pouch.
3. Label pouches and package one (1) pouch in each shipper box. Label shipper boxes.

### Example 13: Sheep Study

Sheep are accepted as a model of frontal sinus treatment. Apart from monkeys, apes, and swine, the sheep sinus most closely resembles that of humans in terms of anatomy, physiology, and pathology. Sheep were chosen for an antra-sinus study of betamethasone dipropionate cream since they possess nasal cavities, maxillary, ethmoid, and frontal sinuses, and respiratory type sinonasal epithelium that closely resembles humans. Furthermore, sheep sinuses possess complex immune systems with many similarities to humans.

In humans, after topical or intramuscular application, betamethasone dipropionate is metabolized to betamethasone-17-propionate and betamethasone with low levels of betamethasone-21-propionate also reported in some studies.

In humans, after topical administration of betamethasone dipropionate in Servino Spray (FDA PharmReview, NDA 208079), plasma concentrations of betamethasone dipropionate, betamethasone-17-dipropionate, and betamethasone were measured at baseline, and before and after the last dose in 75 subjects with psoriasis receiving topical betamethasone dipropionate 0.05% spray or lotion BID for 15 days. The majority of subjects had no measurable plasma concentration of betamethasone dipropionate (<5 pg/mL). Both betamethasone and betamethasone-17-propionate were present at plasma concentrations up to 120 μg/mL.

A non-GLP study of BMDP CREAM (0.05% betamethasone) was conducted in sheep. Sheep were selected for this study as they possess nasal cavities, maxillary, ethmoid, and frontal sinuses, and respiratory type sino-nasal epithelium that are similar to the human sinus, and they possess complex immune systems with many similarities to humans (Ha 2007; Le 2008; Rajiv 2013; Drilling 2014; Ooi 2018). Although a novel route of administration, the study design is consistent with typical toxicology study designs and the principles described in ICH M3(R2). The study was conducted in accordance with ISO:9001 (2015) quality management system guidelines, as well as the Study Plan and the Test Facility standard operating procedures (SOPs). Appropriate animal ethics approval was obtained. Results are described below.

The objective of this study was to evaluate the potential local tolerance and systemic absorption of BMDP CREAM following a single dose intra-sinus administration and a 10-day recovery period. The intra-sinus route of administration is the intended clinical route of administration. During the study, the animals (n=6 castrated males; 15-16 months of age) were examined for clinical signs of toxicity, local tissue response, clinical pathology, pharmacodynamic response (serum cortisol and glucose) and histopathology evaluations of selected tissues and determination of systemic exposure to betamethasone and betamethasone-17-propionate. The control article was 0.9% saline.

On Day 0, under general anesthesia, sinus access was obtained via bilateral frontal trephination with placement of small metal cannulas through a drilled hole into both frontal sinuses to enable access for Test and Control Items injection. Fluorescein flush through the trephines was employed to check successful access and verified by endoscopy. During surgery, BMDP CREAM or saline were administered directly into one frontal sinus, randomised such that each animal received both BMDP CREAM and saline in contralateral sinuses. The whole frontal sinus was filled (between 7 and 15 mL/side) until BMDP CREAM or saline appeared in the nasal cavity (verified by endoscopy). After administration of the test articles, the trephines were removed, and the skin sutured closed over the drill holes.

Body weights were recorded prior to test-article administration surgery and then once weekly for the duration of the study. Specific observations of nasal discharge were made twice daily, at least 6 hr apart, throughout the study. The color and texture of fluid and estimated volume were recorded.

Blood was collected (via a jugular vein cannula inserted during surgery) during the 10-day post-dose phase for pharmacokinetics, hematology, clinical chemistry, glucose, and morning cortisol analysis. Pharmacokinetic bioanalysis was conducted on stabilised plasma using a qualified LC/MS/MS method with a lower limit of quantification of 0.02 ng/mL for betamethasone 17-propionate and betamethasone.

On Day 10, animals were sacrificed, with a gross pathology examination conducted and selected tissues collected for microscopic examination. Tissues collected included the frontal sinuses, nasopharynx, esophagus, rumen, duodenum, brain, heart, lung, liver, kidney, and spleen.

The volume of BMDP CREAM (0.05% betamethasone) instilled into the sinus cavity reflected the variability in the sinus volume of sheep. Based on a cream density of 0.8 g/mL and body weight, the individual dose of BDMP CREAM and betamethasone was determined.

All sheep recovered well from surgery, with no adverse clinical signs of toxicity noted throughout the 10-day recovery period. One animal exhibited a clear discharge following surgery with sneezing on Day 2 post-dose, but no other signs were noted in this animal. Two animals had approximately 2% or 6% body weight loss by the termination of the study. All other sheep maintained or gained weight during the study.

Plasma levels of betamethasone and betamethasone 17-proprionate were detected through 3 days post-dose, and the plasma concentration versus time curve reflected the metabolism of betamethasone dipropionate to betamethasone. Maximum plasma levels of betamethasone were observed at about 24 hr post-dose.

Briefly, betamethasone dipropionate cream (0.05% betamethasone, density 0.78) of Example 8 was administered via the intra-sinus route to sheep, and the plasma levels of active metabolites were measured. The betamethasone dipropionate cream doses were delivered to fill one sheep sinus. Because of the variability in the volume of the sinus in sheep, the total volume varied from 5 to 15 mL. The corresponding doses of betamethasone dipropionate (and the calculated betamethasone dose) are provided in Table 33. Doses calculated by body weight and body surface area (BSA) are also shown.

TABLE 33
Dose Administered to Sheep Frontal Sinus During Surgery

| | | Volume of | | | | |
|---|---|---|---|---|---|---|
| | | BMDP | | | | |
| | | Cream | Betamethasone | | | |
| | Sheep | Admin- | Dipropionate | | | |
| Sheep | Weight | istered | Dose | Betamethasone Dose | | |
| ID | (kg) | (mL) | (mg) | mg | μg/kg | mg/m²* |
| 1 | 60 | 15 | 7.8 | 6.0 | 100 | 3.7 |
| 2 | 57 | 5 | 2.6 | 2.0 | 35 | 1.3 |
| 3 | 52 | 10 | 5.2 | 4.0 | 77 | 2.8 |
| 4 | 59 | 10 | 5.2 | 4.0 | 68 | 2.5 |
| 5 | 55 | 12 | 6.2 | 4.8 | 87 | 3.2 |
| 6 | 55 | 8.5 | 4.4 | 3.4 | 62 | 2.3 |
| Mean | 56.3 | 10.1 | 5.2 | 4.0 | 71.5 | 2.6 |

*Determined based on (FDA 2005) [https://www.fda.gov/media/72309/download; Guidance for Industry: Estimating the Maximum Safe Starting Dose in Initial Clinical Trials for Therapeutics in Adult Healthy Volunteers. U.S. Department of Health and Human Services, Food and Drug Administration, Center for Drug Evaluation and Research (CDER), July 2005, Pharmacology and Toxicology.]

It should be noted that systemic absorption from a mucosal surface will be greater than from dermal application due to the stratum cutaneous epithelial barrier of the skin compared with the thinner and more vascularized mucosal surface.

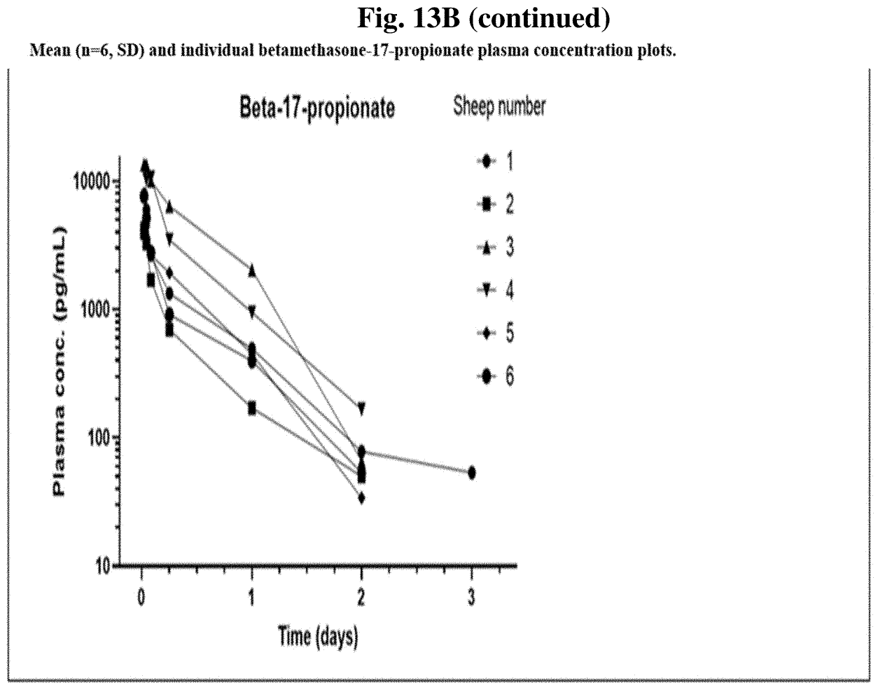

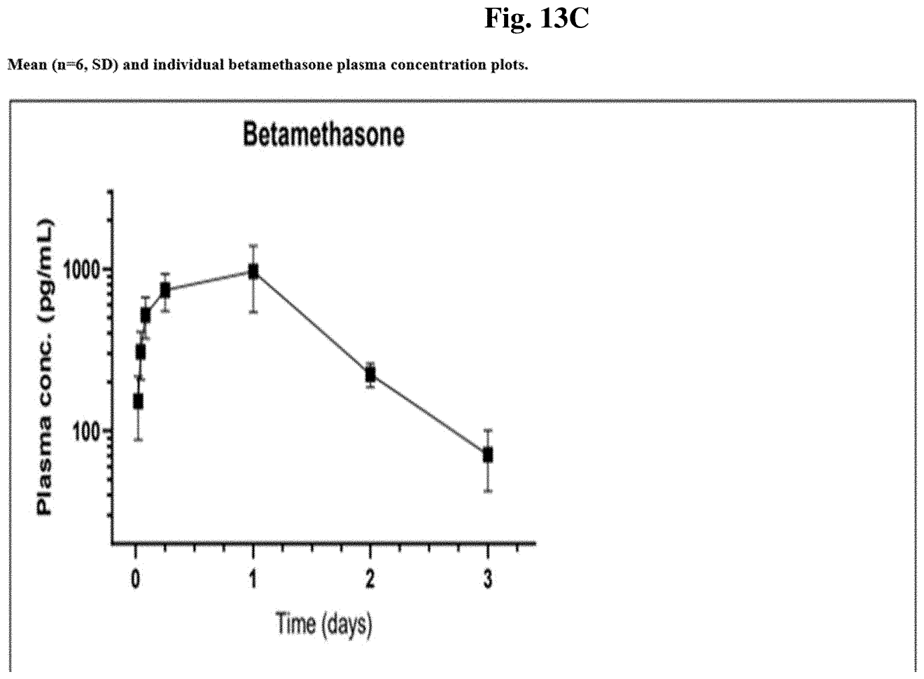

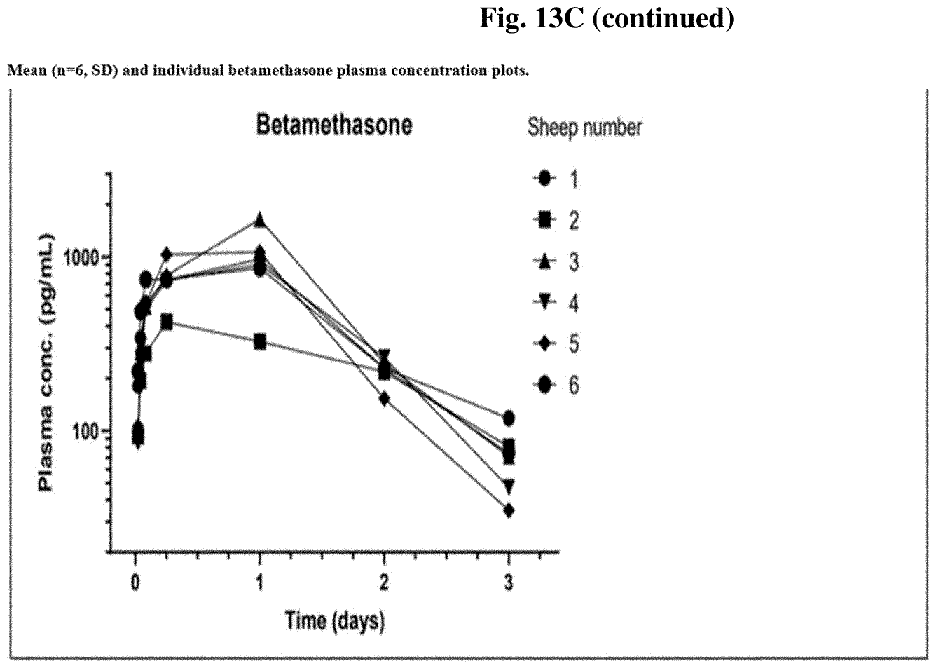

The concentration of betamethasone and betamethasone-17-propionate in plasma were measured and the resulting mean concentration-time curves and pharmacokinetic parameters are shown in FIGS. 13A-C. Overall and individual data are provided in Tables 34-36 below.

TABLE 34
Pharmacokinetic Parameters of Betamethasone and Betamethasone
Dipropionate in Sheep Treated with Intra-Sinus 0.05% Betamethasone
Dipropionate Cream (Mean (SD, n = 6))

| | | Betamethasone |
|---|---|---|
| | Betamethasone | 17-propionate |
| Cₘₐₓ (pg/ml) | 980 ± 397 | 8,279 ± 3,376 |
| Tₘₐₓ (hr) | 24 (6-24) | 0.5 (0.5-2) |
| AUCₗₐₛₜ (ng/mL*hr) | 33.5 ± 8.9 | 58.3 ± 44.7 |
| AUC_{inf} (ng/ml*hr) | 35.2 ± 8.3 | 59.3 ± 44.8 |
| Apparent half-life (hr) | 14.9 ± 7.0 | 9.5 ± 2.2 |

Median (range) for Tmax.

TABLE 35
Individual Betamethasone-17-Propionate Plasma Concentrations (pg/mL)
(Mean +/− SD (n = 6)) (BLQ = below limit of quantitation)
Individual betamethasone-17-propionate plasma concentrations (pg/mL)

| Sample time | | Sheep number | | | | | | | |
|---|---|---|---|---|---|---|---|---|---|
| Hours | Days | 1 | 2 | 3 | 4 | 5 | 6 | Mean | SD |
| 0 | 0 | 0 | 0 | 0 | 0 | 0 | 0 | 0 | 0 |
| 0.5 | 0.021 | 4433 | 3938 | 13420 | 4290 | 7997 | 7623 | 6950 | 3630 |
| 1 | 0.042 | 5984 | 3267 | 13310 | 10362 | 4477 | 5148 | 7091 | 3894 |
| 2 | 0.083 | 2651 | 1694 | 10032 | 10714 | 2651 | 2772 | 5086 | 4120 |
| 6 | 0.25 | 1331 | 696.3 | 6391 | 3487 | 1925 | 909.7 | 2457 | 2172 |
| 24 | 1 | 493.9 | 170.5 | 2035 | 944.9 | 449.9 | 398.2 | 749 | 679 |
| 48 | 2 | 77.9 | 50.2 | 65.8 | 166.1 | 34.0 | 53.2 | 74.5 | 47 |
| 72 | 3 | 53.4 | BLQ | BLQ | BLQ | BLQ | BLQ | | |
| 120 | 5 | BLQ | BLQ | BLQ | BLQ | BLQ | BLQ | | |

BLQ = below limit of quantitation

TABLE 36
Individual Betamethasone Plasma Concentrations (pg/mL)
(Mean +/− SD (n = 6)) (BLQ = below limit of quantitation)
Individual betamethasone plasma concentrations (pg/mL)

| Sample time | | Sheep number | | | | | | | |
|---|---|---|---|---|---|---|---|---|---|
| Hours | Days | 1 | 2 | 3 | 4 | 5 | 6 | Mean | SD |
| 0 | 0 | 0 | 0 | 0 | 0 | 0 | 0 | 0 | 0 |
| 0.5 | 0.021 | 181.5 | 93.3 | 224.4 | 85.6 | 106.4 | 220.0 | 151.9 | 64.3 |
| 1 | 0.042 | 339.9 | 193.6 | 310.2 | 254.1 | 259.6 | 486.2 | 307.3 | 101.1 |
| 2 | 0.083 | 541.2 | 278.3 | 515.9 | 497.2 | 535.7 | 741.4 | 518.3 | 147.4 |
| 6 | 0.25 | 735.9 | 421.3 | 782.1 | 732.6 | 1028.5 | 741.4 | 740.3 | 193.2 |
| 24 | 1 | 971.3 | 325.6 | 1650.0 | 907.5 | 1064.8 | 863.5 | 963.8 | 424.7 |
| 48 | 2 | 228.8 | 218.9 | 244.2 | 264.0 | 152.9 | 228.8 | 222.9 | 37.8 |
| 72 | 3 | 117.7 | 80.9 | 71.3 | 47.2 | 34.9 | 74.3 | 71.0 | 28.8 |
| 120 | 5 | BLQ | BLQ | BLQ | BLQ | BLQ | BLQ | | |

BLQ = below limit of quantitation

The variability (e.g. Coefficient of Variation=40% for Cmax) in pharmacokinetics could not be correlated with the administered dose (which varied by 3-fold), suggesting that there will be variable absorption of this product at this site. AUC was more consistent, with a CV of 25%.

Following dose administration, plasma glucose levels increased by about 30% on Day 1 but then returned to pre-dose levels by Day 2. Serum cortisol decreased to basal levels (less than 10 nmol/L) at Day 1 and remained lower than pre-dose levels through the 10-day study. The lower cortisol levels and Day 1 glucose levels are consistent with the pharmacological activity of glucocorticoids. The prolonged cortisol suppression is likely due to the prolonged plasma levels of betamethasone and betamethasone 17-proprionate.

No steroid-related changes were apparent for other clinical chemistry or hematology parameters compared to pre-dose values.

At necropsy, one animal had a thickened sinus and a significant bacterial infection. No other gross findings were noted in the other sheep. Also, no irritation of the sinus was observed. Histopathological evaluation revealed evidence of infection in one sheep that resulted in inflammatory cell infiltration, and ciliary denudation in some mucosal specimens in both BMDP CREAM and saline-treated sinuses. There was no correlation of local toxicity with either Test or Control item treatment. No histopathology changes were observed in the heart, lungs, liver, kidneys, or spleen.

Overall, intra-sinus instillation of BMDP CREAM to the frontal sinus mucosa did not appear to induce local toxicity or inflammation or evidence of adverse pathology.

Because of the variability of the sinus in sheep, the dose of betamethasone for calculating a Human Equivalent Dose is expressed in terms of total body surface area (BSA) as described in FDA Guidance (FDA 2005). The mean dose in the sheep study was 0.072 mg/kg and can be converted to dose per BSA based on a calculated Kₘ for sheep of 37. It is noteworthy that the Kₘ for sheep and humans is the same value. The calculated mean dose in the sheep study is therefore 2.6 mg/m² (range 1.3 to 3.7 mg/m²). In the planned clinical trial, a patient will be treated with a total maximum dose 10 mL equivalent to 4.0 mg betamethasone, equivalent to a dose of 0.067 mg/kg for a 60 kg individual. On a BSA basis, this dose equates to approximately 2.5 mg/m².

Sheep have been used as preclinical models for steroids including betamethasone compounds, and the metabolism and pharmacokinetics of these drugs is similar in the two species (9, 11, 12). The current data suggests that the metabolism of betamethasone dipropionate from intra-sinus administration may be predictive of human metabolism by this route of administration.

The sheep weights were relatively consistent, varying between 52 and 60 kg, with a mean of 56.3 kg. This is similar to the nominal human weight of 60 kg as defined by the FDA for normalised human dose calculations.

The mean volume of cream delivered to the sheep was 10 mL. The resulting betamethasone dose in the sheep was 0.072 mg/kg or 2.6 mg/m².

The volume of the human frontal sinus is reported to be variable and ranges from 2 mL to 10 mL.

A human dose of 10 mL of the same formulation is equivalent to 4.0 mg betamethasone, equivalent to a dose of 0.067 mg/kg for a 60 kg individual. On a BSA basis, this dose equates to approximately 2.5 mg/m².

Thus, the dose proposed for use in the clinic is nearly identical to the dose used in the sheep study.

Since the metabolism and pharmacokinetics of betamethasone products are similar between sheep and humans, and the sinus mucosa is also similar, and the body weights and surface areas are similar, it is likely, therefore, that the resulting absorption profile in humans treated with intra-sinus BMDP will be similar.

### Example 14: Phase 1 Study in Humans

25 post-FESS patients, aged 18 to 80 years of age diagnosed with chronic rhinosinusitis (CRS) with uncontrolled symptoms ongoing at least 30 days will be enrolled. All patients must have had a previous FESS procedure at least 6 months before enrollment in the study. The first 6 patients will return daily to observe cream retention until cream is no longer visible via nasal endoscope.

Up to 5 mL of 0.05% betamethasone dipropionate cream as described in Example 12 will be placed onto each of the left and right sinus mucosa (total of 10 ml). The cream will be applied topically onto the inflamed sinus mucosa using a custom-designed applicator attached to a syringe with the aid of a nasal endoscope.

During the study, all patients will return 5 days after treatment for safety assessment and return again at 21 days after treatment for the exit visit.

During the study, morning cortisol levels, intraocular pressures and adverse events will be measured pretreatment, at day 5 post-treatment and at the exit visit.

During the study, changes in the average daily total symptom score of the 7-days of the screening run-in period using the 4-Cardinal Symptoms Score (4CSS) Daily Diary versus the 7-day average daily total score for the 7-days prior to the exit visit. The 4CSS is a composite score of the cardinal symptoms of CRS for patients with CRS scored 0.3—with a total score of 12. The four “cardinal” symptoms are: (1) obstruction and congestion; (2) facial pain and pressure; (3) nasal discharge; and (4) olfactory loss (loss of sense of smell).

During the study, changes in total SNOT-22 scores between pre-treatment and day 21 post-treatment will be measured.

During the study, changes in 4CSS VAS scores will be assessed pre-treatment versus day 21 post-treatment.

During the study, changes in Modified Lund Mackay Endoscopy Scores (pre-treatment versus day 21) based on video assessments by three independent, blinded ENTs will be assessed.

During the study, changes in cream retention time in sinuses will also be measured for the initial six patients. Patients will return until cream is no longer visible via endoscope.

This is a prospective, open-label, single-site clinical study of the safety, tolerability and preliminary efficacy of BMDP CREAM applied to the sinus mucosa in post-FESS patients, 18 to 80 years of age, diagnosed with uncontrolled chronic rhinosinusitis symptoms. To warrant the diagnosis of uncontrolled post-FESS chronic rhinosinusitis, a patient must have been previously diagnosed with chronic rhinosinusitis and have been actively treated for rhinosinusitis symptoms which have been persistent for the previous 30 days. A FESS procedure must have been performed no less than 6 months prior to screening. See Schedule of Activities for details of study activities.
Patients will complete a 4CSS questionnaire and must have a score ≥2 on at least two of the “Cardinal” symptoms at screening (one symptom must be Obstruction and Congestion) to qualify for enrollment
After the screening assessment, enrolled patients undergo a 7-day run-in screening period where they will continue utilizing their current treatment regimen
At the screening, enrolled patients will receive the 4CSS Daily Diary that will be completed during the 7-day run-in screening period
On the day of treatment visit, patients will report their 4-Cardinal Symptoms and patients that do not score ≥2 on at least two of the “Cardinal” symptoms (one symptom must be Obstruction and Congestion) are exited from the study and not considered to be uncontrolled with available treatments
On the treatment day (prior to application of treatment) and at the exit visit, video of the patient's sinus mucosa will be recorded for independent assessment of inflammatory burden
Patients will complete a VAS assessment of symptom burden as per the study plan
Patients will complete the SNOT-22 assessment as per the study plan
Intraocular pressure assessment (IOPs) will be measured at all visits
Treated patients are dosed one-time in office by placing up to 10 mL of BMDP CREAM onto inflamed sinus mucosa with the aid of an endoscope. If 10 mL cannot be inserted due to sinus structure, actual dose will be recorded via weight of syringe(s) before and after
Patients will receive the 4-Cardinal Symptoms Score Daily Diary that will be completed daily until the exit visit. A composite of the 7 days prior to the exit visit will be the primary measure of CRS symptom improvement
Patients will stop using their regular CRS treatment regimen on the night before scheduled treatment visit and resume regular treatments after they return for a safety assessment, 5-days after treatment
Patients return to the clinic 21 days after treatment for evaluation, safety assessment, and study exit
The first six patients of the planned 25 patient enrollment will participate in a cream retention arm of the study. These six patients return daily until cream is no longer visible in the sinus cavities
Assessments:
   Morning cortisol will be measured prior to treatment, on Day 5 and at Day 21 (exit visit) for all patients.
   Fasting glucose levels will be measured prior to treatment, on Day 5 and Day 21 for the first six patients.
   Cream retention will be measured daily for the first six patients until no longer visible (and in all patients at day 5 and day 21

Inclusion Criteria:
1. Healthy adults, 18-80 years of age
2. Patients who have undergone functional endoscopic sinus surgery at least 6 months prior to enrolment
3. Clinically confirmed diagnosis of chronic rhinosinusitis
4. At least 1 trial of topical corticoid sprays or irrigations for a minimum of 1 month prior to screening without adverse effects.
5. Able to provide informed consent and comply with study conditions
6. Females of childbearing potential must use adequate birth control methods and not plan to get pregnant during the course of the study
7. Patients who are stable on other non-steroidal medications.
8. Patients must have a score ≥2 on at least two of the “Cardinal” symptoms at screening (one symptom must be Obstruction and Congestion) to qualify for enrolment to study and treatment.

Exclusion Criteria:
1. Pregnant or breastfeeding females
2. Patients who have undergone any sinus surgery within 6 months prior to enrolment
3. Acute sinusitis
4. Uncontrolled asthma
5. History or current glaucoma or cataract
6. Allergies or contraindications to betamethasone dipropionate, corticosteroids or topical anaesthesia
7. Sino-nasal abnormalities, disease or implanted devices that prevent application of the therapy
8. Previous enrolment in this study
9. Inability to provide informed consent or comply with study protocol
10. If they have abnormal IOP at screening or pretreatment (abnormal IOP is defined as greater than 21 mm Hg)
11. Diabetes

For the 4-CSS diary, the ratings will be “None,” “Mild,” “Moderate,” or “Severe” for “Obstruction and Congestion,” “Facial Pain and Pressure,” “Nasal Discharge,” and “Loss of Sense of Smell.” The VAS will have the patients rate total sinus symptoms, nasal blockage, headache/pressure on the face, loss of smell, post-nasal drip (secretions from the nose down to the throat), runny nose, itchy eyes, itchy nose, sneezing, tearing, cough, tightness/pressure sensation on the chest, shortness of breath/difficulty with breathing, and wheezing from “None” to “More than I can imagine.” The SNOT-22 will have ratings of “No Problem (0),” “Very Mild Problem (1),” “Mild or slight Problem (2),” “Moderate Problem (3),” “Severe Problem (4),” or “Problem as bad as it can be (5)” for need to blow nose, nasal blockage, sneezing, runny nose, cough, post-nasal discharge, thick nasal discharge, ear fullness, dizziness, ear pain, facial pain/pressure, decreased sense of smell/taste, difficulty falling asleep, wake up at night, lack of a good night's sleep, wake up tired, fatigue, reduced productivity, reduced concentration, frustrated/restless/irritable, sad, and embarrassed, with an addition field for which symptoms are the most important (maximum of 5).

### Example 15: Phase 2 Study in Humans

A Phase 2 randomized, double-blind, multicenter, placebo-controlled, single-dose safety, pharmacokinetic and efficacy study of betamethasone dipropionate (equivalent to 0.05% w/w betamethasone) cream for the treatment of chronic rhinosinusitis in patients who have previous undergone FESS will be performed.

60 randomized patients (1:1 active:placebo), aged 18 to 80 years of age diagnosed with chronic rhinosinusitis with uncontrolled symptoms ongoing at least 30 days and having previously undergone a FESS procedure at least 6 months prior to enrollment will be enrolled.

This is a prospective, randomized, double-blinded, multicentre, placebo-controlled, clinical study of the efficacy and safety of BMDP CREAM applied to the sinus mucosa in post-FESS patients, 18 to 80 years of age, diagnosed with uncontrolled CRS symptoms. To warrant the diagnosis of uncontrolled post-FESS CRS, a patient must have been previously diagnosed with CRS and have been actively treated for rhinosinusitis symptoms that have been persistent for the previous 30 days. A FESS procedure must have been performed no less than 6 months prior to screening.

Patients will complete a 4CSS questionnaire and must have a score ≥2 on at least two of the “Cardinal” symptoms at screening (one symptom must be Obstruction and Congestion) to qualify for enrolment.

After the screening assessment, enrolled patients undergo a 7-day run-in screening period during which they will continue utilizing their current treatment regimen.

At screening, enrolled patients will receive the 4CSS Daily Diary that must be completed at-home daily during the 7-day run-in screening period.

During the treatment visit, patients will report their 4-Cardinal Symptoms and patients that do not score ≥2 on at least two of the “Cardinal” symptoms (one symptom must be Obstruction and Congestion) are screening failures.

Prior to treatment and at the exit visit, a video of the patient's sinus mucosa will be recorded for independent assessment of inflammatory burden.

Patients will complete a VAS assessment of symptom burden.

Enrolled patients are dosed one-time in office (or in a clinical research unit, CRU). Patients will receive the 4-Cardinal Symptoms Score Daily Diary that will be completed daily until the exit visit. A maximum of 5 mL of betamethasone dipropionate cream will be placed onto the left and right inflamed sinus mucosa (total of 10 mL). The cream will be prefilled into a syringe at manufacture and will be clinician-administered topically onto the inflamed sinus mucosa via an applicator attached to the syringe. Placement will be done with the aid of a nasal endoscope

Patients will stop use of their regular sinusitis treatment regimen after the application of BETA CREAM and resume regular treatments 5-days after treatment.

Patients return to the clinic 21 days after treatment for evaluation, safety assessment, and study exit.

Inclusion Criteria:
12. Healthy adults, 18-80 years of age
13. Patients who have undergone functional endoscopic sinus surgery at least 6 months prior to enrolment
14. Clinically confirmed diagnosis of chronic rhinosinusitis
15. At least 1 trial of topical corticoid sprays or irrigations for a minimum of 1 month prior to screening without adverse effects.
16. Able to provide informed consent and comply with study conditions
17. Females of childbearing potential must use adequate birth control methods and not plan to get pregnant during the course of the study
18. Patients who are stable on other non-steroidal medications.
19. Patients must have a score ≥2 on at least two of the “Cardinal” symptoms at screening (one symptom must be Obstruction and Congestion) to qualify for enrolment to study and treatment.

Exclusion Criteria:
20. Pregnant or breastfeeding females
21. Patients who have undergone any sinus surgery within 6 months prior to enrolment
22. Acute sinusitis
23. Uncontrolled asthma
24. History or current glaucoma or cataract
25. Allergies or contraindications to betamethasone dipropionate, corticosteroids or topical anaesthesia
26. Sino-nasal abnormalities, disease or implanted devices that prevent application of the therapy
27. Previous enrolment in this study
28. Inability to provide informed consent or comply with study protocol
29. If they have abnormal IOP at screening or pretreatment (abnormal IOP is defined as greater than 21 mm Hg)
30. Diabetes

Primary Objectives:

Safety:

Comparison of adverse events in active treatment group with adverse events in the placebo group.

Pharmacokinetics:

PK analysis will be performed on a subset of patients enrolled in this study.

Efficacy:

Based on Study OT-007 clinical results and regulatory discussions, one of the following potential endpoints will potentially be utilized as the primary efficacy endpoint and the other potential endpoints will be used as secondary or exploratory endpoints:
Change in the average daily total symptom score of the 7-days of the screening run-in period using the 4-Cardinal Symptoms Score (4CSS) Daily Diary versus the 7-day average daily total score for the 7-days prior to the exit visit. The 4CSS is a composite score of the cardinal symptoms of CRS for patients with CRS scored 0-3 with a total score of 12. The four “cardinal” symptoms are: (1) obstruction and congestion; (2) facial pain and pressure; (3) nasal discharge; and (4) olfactory loss (loss of sense of smell).
Change in total SNOT-22 scores between pre-treatment and day 21 post-treatment.
Change in 4CSS VAS scores between pre-treatment and day 21 post-treatment.
Change is Modified Lund Mackay Endoscopy Scores (pre-treatment versus day 21) based on video assessments by three independent, blinded ENTs.

Exploratory Endpoints:
BMDP CREAM related adverse events
BMDP CREAM application-related adverse events

### Example 16: Stability Testing

Betamethasone dipropionate (0.05%) creams were prepared as described in Table 28 except using 1.75% glycerin and stored at 25° C./60% Relative Humidity (RH) (Sample #1), 30° C./65% RH (Sample #2) or 40° C./75% RH (Sample #3) for one month or three months. The betamethasone dipropionate (BMDP) and betamethasone (BA) content were measured at the start of storage and at the one month or three month interval, respectively. pH was also measured for a neat preparation and a 1:5 dilution at the start of storage and at the one month or three month interval, respectively. Particle size and globule size were likewise measured at the start of storage and at the one month or three month interval, respectively, according to USP 729. Impurities were also measured at the start of storage and at the one month or three month intervals, respectively. Viscosity was also measured at the start of storage and at the one month or three month intervals, respectively. Osmolality was also measured at the start of storage and at the one month or three month intervals, respectively, according to USP 785.

The results of the stability study are provided in Table 40-46.

Briefly, to measure betamethasone dipropionate and betamethasone content as well as impurities/degradants, HPLC was used. Samples were prepared in duplicate. 2 g of cream was weighed into a 50 mL centrifuge tube, 3.0 mL of diluent (ethanol) was added to the tube and 3.0 mL of IS Working Stock Solution was also added to the tube. IS Working Stock Solution was prepared from a IS Stock Solution by weighing approximately 16.7 mg of prednisone reference standard into a 50 mL volumetric flask and dissolving the prednisone reference standard to volume with diluent (ethanol) with sonication as needed to dissolve with mixing to obtain the IS Stock Solution; 12.0 mL of the IS Stock Solution was then pipetted into a 100 mL volumetric flask and diluted to volume with diluent (ethanol) to yield the IS Working Stock Solution. The 50 mL tubes were then vortexed for about 30 seconds and placed in a 70° C. water bath for 15 minutes to dissolve the cream with intermittent cortexing after approximately 7 minutes. The 50 mL tubes were then removed from the heat and vortex mixed again for 30 seconds. If necessary, the tubes were returned to the water bath to prevent cooling. The tubes were then shaken for 20 minutes and placed in the freezer for 15 minutes to allow the petrolatum from the cream to solidify in the tube. The tubes were then centrifuged for 30 minutes at 12,000 RPM and supernatant was transferred to a HPLC vial for analysis.

The HPLC system was rinsed well with 50:50 acetonitrile:water to remove buffer salts after each run. In some instances, a needle wash of 100% ethanol was used. HPLC was run using a Hypersil ODS 10×30 mm, 3 μm column as the Guard Column and a Hypersil ODS 3×150 mm, 3 μm column as the Analytical Column. The column temperature was maintained at 35° C. with a runtime of 45 minutes, a flow rate of 0.5 mL/minute, an injection volume of 3 μL, an autosampler temperature of 30° C., and using a 254 nm (UV absorbance) detector and collecting PDA from 200 nm to 400 nm for identification testing.

Mobile Phase A was prepared as 88:12 buffer:acetonitrile with buffer being prepared from 6.6 g of ammonium phosphate dibasic in 1 L of water with the pH adjusted to 7.00+/−0.05 using phosphoric acid. Mobile Phase B was prepared as 88:12 methanol:acetonitrile. Mobile Phase C was prepared as 30:5:65 buffer:methanol:acetonitrile. All mobile phase solutions were mixed well and degassed prior to use. In addition, a betamethasone dipropionate stock standard solution (BD Stock) was prepared by weighing approximately 33.4 mg of betamethasone dipropionate reference standard into a 25 mL volumetric flask, dissolving in diluent to volume, sonicating to dissolve and mixing well. A Working Standard Solution was prepared from 3.0 mL of the IS Stock Solution and 8.0 mL of the BD Stock in a 50 mL volumetric flask to which 150 mg of benzyl alcohol reference standard was added and diluent added to volume followed by mixing well. A Sensitivity Solution was prepared from 5.0 mL of the Working Standard Solution in a 100 mL volumetric flask and diluting to volume with diluent followed by mixing well and then pipetting 1.0 mL of the resulting solution into a 100 mL volumetric flask and diluting to volume with diluent and mixing well.

A Peak ID standard was prepared by weighing 5 mg of each impurity standard into separate 100 mL volumetric flasks, dissolving completely to volume with diluent and then diluting 1.0 mL of each stock impurity solution to 100 mL in a new volumetric flask together and to volume with diluent. The impurities included, betamethasone and betamethasone dipropionate from Sigma-Aldrich, and reference standards for betamethasone 21-acetate-17-propionate, betamethasone 21-propionate, betamethasone dipropionate EP Impurity B, betamethasone dipropionate EP Impurity F, betamethasone dipropionate EP Impurity G, betamethasone dipropionate EP Impurity I, and 6-bromo-betamethasone-17,21-dipropionate.

The gradient program and injection order used are provided in Table 37 below. System suitability requirements included those in Table 38. Peak identification parameters are provided in Table 39 below.

The Ratio RT (RRT) in Table 39 can be calculated from the ratio of the sample retention time to the mean retention time of the bracketing standards. The % LC can be calculated as the peak area response ratio in the sample multiplied by the weight of reference standard (in mg) multiplied by the purity of the reference standard (in decimal form) multiplied by the dilution of standard solution multiplied by the volume of sample solution (in mL) multiplied by 100 divided by the mean peak area response ratio in the bracketing standards divided by the volume of standard solution (in mL) divided by the weight of sample (in mg) divided by the label claim of the sample (as % w/w/100%). The % of related substances can be calculated as the related substance peak area in the sample injection multiplied by 100 divided by the peak area of betamethasone dipropionate in the sample injection divided by the relative response factor of the related substance (assumed to be 1.0).

TABLE 37
Gradient Program and Injection Order

| | | % Mobile | % Mobile | % Mobile |
|---|---|---|---|---|
| Gradient Program | Time (min) | Phase A | Phase B | Phase C |
| | 0.0 | 71.6 | 28.4 | 0 |
| | 1.8 | 48.9 | 51.1 | 0 |
| | 10.8 | 31.8 | 68.2 | 0 |
| | 22 | 31.8 | 68.2 | 0 |
| | 23.3 | 0 | 0 | 100 |
| | 38.5 | 0 | 0 | 100 |
| | 38.6 | 71.6 | 28.4 | 0 |
| | 45 | 71.6 | 28.4 | 0 |

| Solution | # of Injections |
|---|---|
| Diluent | 3 |
| Sensitivity | 1 |
| Peak ID | 1 |
| Placebo ID | 1 |
| Working Standard (Precision) | 5 |
| Check Standard | 2 |
| Working Standard (Calibration) | 1 |
| Sample 1 Preparation 1 | 1 |
| Sample 1 Preparation 2 | 1 |
| Sample 2 Preparation 1 | 1 |
| Sample 2 Preparation 2 | 1 |
| Sample 3 Preparation 1 | 1 |
| Sample 3 Preparation 2 | 1 |
| Working Standard (Calibration) | 1 |
| Etc. | |

TABLE 38
System Suitability Requirements

| | |
|---|---|
| Blank | The last injection of diluent does not show any peaks that |
| interference | would interfere with the benzyl alcohol, betamethasone |
| | dipropionate, or internal standard peaks. Interference is |
| | defined as a response that is greater than 0.2% when |
| | compared to the corresponding peak area in the mean of |
| | the system suitability standard solutions. |
| Sensitivity | The signal to noise of the betamethasone dipropionate |
| | peak is NLT 10. |
| Repeatability | NMT 2.0% RSD for the benzyl alcohol and |
| | betamethasone dipropionate peak areas in 5 replicate |
| | system suitability standard injections. |
| Standard Check | 98.0-102.0% for benzyl alcohol and betamethasone |
| | dipropionate in the check standard |
| USP Tailing | NMT 1.8 |
| Factor | |
| Standards Over | NMT 2.0% RSD for the benzyl alcohol and |
| the Run | betamethasone dipropionate peak areas for all standard |
| | A injections throughout the run |

TABLE 39
Peak Identification
Peak Identification

| | | ~RT | ~RRT | |
|---|---|---|---|---|
| Label | Compound Name | (minutes) | (BMDP) | RRF |
| — | Placebo Related | 2.154 | 0.14 | — |
| — | Placebo Related | 2.872 | 0.18 | — |
| — | Benzyl Alcohol | 3.848 | 0.24 | — |
| — | BA-2 | 4.986 | 0.31 | — |
| — | Placebo Related | 5.411 | 0.34 | — |
| — | Prednisone (Internal Standard) | 6.185 | 0.39 | — |
| 1 | Betamethasone | 8.093 | 0.50 | 1.00 |
| 5 | Betamethasone 17-propionate | 11.465 | 0.71 | 1.00 |
| | (Impurity B) | | | |
| 4 | Betamethasone 21-propionate | 11.890 | 0.74 | 1.00 |
| 3 | Betamethasone 21-acetate 17- | 13.982 | 0.87 | 1.00 |
| | propionate | | | |
| — | Betamethasone Dipropionate | 16.050 | 1.00 | — |
| 6 | Betamethasone dipropionate | 16.348 | 1.02 | 1.00 |
| | (Impurity F) | | | |
| 8 | Betamethasone dipropionate | 17.165 | 1.07 | 1.00 |
| | (Impurity I) | | | |
| 2 | Betamethasone dipropionate | 17.658 | 1.10 | 1.00 |
| 7 | Betamethasone dipropionate | 19.577 | 1.22 | 1.00 |
| | (Impurity G) | | | |
| 9 | 6-Bromo-Betamethasone-17-21- | 20.394 | 1.27 | 1.00 |
| | dipropionate | | | |

TABLE 40
Betamethasone Dipropionate (BMDP)
and Betamethasone (BA) Content

| | | | | 1 | 3 |
|---|---|---|---|---|---|
| Lot # | Spec. | Prep. | Initial | Month | Months |
| DDI09Dec2020p28 | 90.0- | 1 | % BMDP | % BMDP | % BMDP |
| | 110.0% | | 99.4 | 100.2 | 99.8 |
| | LC | | % BA | % BA | % BA |
| | | | 97.4 | 94.3 | 91.6 |
| | | 2 | % BMDP | % BMDP | % BMDP |
| | | | 98.5 | 97.1 | 96.2 |
| | | | % BA | % BA | % BA |
| | | | 97.2 | 94.4 | 91.7 |
| | | Mean | % BMDP | % BMDP | % BMDP |
| | | | 99.0 | 98.7 | 98.0 |
| | | | % BA | % BA | % BA |
| | | | 97.3 | 94.4 | 91.7 |
| DDI09Dec2020p28 | 90.0- | 1 | % BMDP | % BMDP | % BMDP |
| Sample #2 | 110.0% | | 99.4 | 100.1 | 98.6 |
| | LC | | % BA | % BA | % BA |
| | | | 97.4 | 93.1 | 88.8* |
| | | 2 | % BMDP | % BMDP | % BMDP |
| | | | 98.5 | 96.2 | 94.8 |
| | | | % BA | % BA | % BA |
| | | | 97.2 | 93.0 | 89.5* |
| | | Mean | % BMDP | % BMDP | % BMDP |
| | | | 99.0 | 98.2 | 96.7 |
| | | | % BA | % BA | % BA |
| | | | 97.3 | 93.1 | 89.2* |
| DDI09Dec2020p28 | 90.0- | 1 | % BMDP | % BMDP | % BMDP |
| Sample #3 | 110.0% | | 99.4 | 99.6 | 97.5 |
| | LC | | % BA | % BA | % BA |
| | | | 97.4 | 90.3 | 79.8* |
| | | 2 | % BMDP | % BMDP | % BMDP |
| | | | 98.5 | 95.0 | 89.9* |
| | | | % BA | % BA | % BA |
| | | | 97.2 | 90.2 | 79.6* |
| | | Mean | % BMDP | % BMDP | % BMDP |
| | | | 99.0 | 97.3 | 93.7 |
| | | | % BA | % BA | % BA |
| | | | 97.3 | 90.3 | 79.7* |

*Out of Specification

TABLE 41
pH Stability for Betamethasone Dipropionate Cream Formulation

| Lot # | Dilution | Initial | 1 Month | 3 Months |
|---|---|---|---|---|
| DDI09Dec2020p28 | 1:5 | 6.7 | 6.7 | 6.7 |
| Sample #1 | Neat | 6.0 | 6.0 | 6.0 |
| DDI09Dec2020p28 | 1:5 | 6.7 | 6.8 | 6.7 |
| Sample #2 | Neat | 6.0 | 6.0 | 6.0 |
| DDI09Dec2020p28 | 1:5 | 6.7 | 6.8 | 6.7 |
| Sample #3 | Neat | 6.0 | 6.0 | 5.9 |

TABLE 42
Particle Size for Betamethasone Dipropionate Cream Formulation (μm)

| | | Initial | | 1 Month | | 3 Months | |
|---|---|---|---|---|---|---|---|
| Lot # | Size | Number | Volume | Number | Volume | Number | Volume |
| DDI09Dec2020p28 | D₁₀ | 0.29 | 1.18 | 0.24 | 1.38 | 0.52 | 0.92 |
| Sample #1 | D₅₀ | 0.79 | 2.75 | 0.60 | 3.55 | 0.94 | 1.53 |
| | D₉₀ | 1.79 | 6.01 | 1.68 | 10.19 | 1.55 | 3.27 |
| | Mean | 0.97 | 3.20 | 0.82 | 5.46 | 1.01 | 1.82 |
| DDI09Dec2020p28 | D₁₀ | 0.29 | 1.18 | 0.24 | 1.76 | 0.47 | 1.30 |
| Sample #2 | D₅₀ | 0.79 | 2.75 | 0.53 | 3.16 | 1.06 | 2.51 |
| | D₉₀ | 1.79 | 6.01 | 2.14 | 10.70 | 2.15 | 6.66 |
| | Mean | 0.97 | 3.20 | 0.89 | 5.22 | 1.23 | 3.37 |
| DDI09Dec2020p28 | D₁₀ | 0.29 | 1.18 | 0.60 | 1.04 | 0.30 | 0.97 |
| Sample #3 | D₅₀ | 0.79 | 2.75 | 0.99 | 1.98 | 0.73 | 1.97 |
| | D₉₀ | 1.79 | 6.01 | 1.71 | 6.68 | 1.49 | 6.98 |
| | Mean | 0.97 | 3.20 | 1.11 | 3.43 | 0.85 | 3.02 |

TABLE 43
Globule Size for Betamethasone Dipropionate Cream Formulation (μm)

| | | Initial | | 1 Month | | 3 Months | |
|---|---|---|---|---|---|---|---|
| Lot # | Size | Number | Volume | Number | Volume | Number | Volume |
| DDI09Dec2020p28 | D₁₀ | 0.61 | 0.99 | 0.63 | 1.04 | 0.63 | 0.87 |
| Sample #1 | D₅₀ | 0.93 | 2.05 | 1.01 | 1.95 | 0.93 | 1.47 |
| | D₉₀ | 1.69 | 7.06 | 1.67 | 10.86 | 1.50 | 3.26 |
| | Mean | 1.08 | 3.23 | 1.12 | 3.73 | 1.02 | 1.88 |
| DDI09Dec2020p28 | D₁₀ | 0.61 | 0.99 | 0.60 | 1.02 | 0.64 | 0.96 |
| Sample #2 | D₅₀ | 0.93 | 2.05 | 0.97 | 2.02 | 0.97 | 1.69 |
| | D₉₀ | 1.69 | 7.06 | 1.69 | 9.82 | 1.60 | 8.60 |
| | Mean | 1.08 | 3.23 | 1.09 | 3.34 | 1.08 | 3.07 |
| DDI09Dec2020p28 | D₁₀ | 0.61 | 0.99 | 0.22 | 1.39 | 0.64 | 0.97 |
| Sample #3 | D₅₀ | 0.93 | 2.05 | 0.59 | 3.30 | 0.98 | 1.75 |
| | D₉₀ | 1.69 | 7.06 | 1.75 | 12.25 | 1.63 | 6.10 |
| | Mean | 1.08 | 3.23 | 0.84 | 5.38 | 1.10 | 2.63 |

TABLE 44A
Impurities for Betamethasone Dipropionate Cream Formulation (stored at 25° C./60% RH)

| | Initial | | | | 1 Month | | | | 3 Month | | | |
|---|---|---|---|---|---|---|---|---|---|---|---|---|
| | | Prep | Prep | | | Prep | Prep | | | Prep | Prep | |
| Impurity | RRT | #1 | #2 | Mean | RRT | #1 | #2 | Mean | RRT | #1 | #2 | Mean |
| BA 2 | 0.26 | 11.63 | 11.07 | 11.35 | | 9.54 | 9.38 | 9.46 | | 6.70 | 6.68 | 6.69 |
| 1 | | | | | | 0.17 | 0.18 | 0.18 | | 0.22 | 0.20 | 0.21 |
| 2 | | | | | | | | | | | 0.10 | 0.10 |
| 3 | 0.84 | ND | 0.21 | 0.21 | | | | | | | | |
| 4 | | | | | | 0.08 | 0.10 | 0.09 | | 0.19 | 0.25 | 0.22 |
| 5 | 0.66 | 0.16 | 0.20 | 0.18 | | 0.31 | 0.37 | 0.34 | | 0.43 | 0.54 | 0.49 |
| 6 | | | | | | | | | | 0.17 | 0.11 | 0.14 |
| 7 | | | | | | | | | | | | |
| 8 | | | | | | | 0.11 | 0.11 | | | | |
| 9 | 1.47 | ND | 0.12 | 0.12 | | | | | | | | |
| Unknown | 0.18 | 0.13 | 0.14 | 0.14 | 0.23 | 0.17 | 0.18 | 0.18 | 0.22 | 0.15 | 0.14 | 0.15 |
| | | | | | 0.47 | 0.05 | 0.05 | 0.05 | 0.43 | 0.05 | 0.07 | 0.06 |
| | | | | | 0.53 | 0.07 | 0.07 | 0.07 | 0.46 | 0.06 | 0.06 | 0.06 |
| | | | | | 0.76 | 0.05 | 0.05 | 0.05 | 0.53 | 0.08 | 0.12 | 0.10 |
| | | | | | 0.89 | 0.10 | 0.35 | 0.23 | 0.76 | 0.09 | 0.09 | 0.09 |
| | | | | | 1.40 | | 0.43 | 0.43 | 0.89 | 0.16 | 0.35 | 0.26 |
| | | | | | 1.44 | | 0.12 | 0.12 | 1.15 | | 0.09 | 0.09 |
| | | | | | 1.47 | | 0.20 | 0.20 | 1.41 | 0.08 | 0.61 | 0.35 |
| | | | | | | | | | 1.45 | | 0.18 | 0.18 |
| | | | | | | | | | 1.49 | | 0.29 | 0.29 |
| Totals | | 0.29 | 0.67 | 0.65 | | 1.0 | 2.21 | 2.05 | | 1.68 | 3.20 | 2.79 |

TABLE 44B
Impurities for Betamethasone Dipropionate Cream Formulation (stored at 30° C./65% RH)

| | Initial | | | | 1 Month | | | | 3 Month | | | |
|---|---|---|---|---|---|---|---|---|---|---|---|---|
| | | Prep | Prep | | | Prep | Prep | | | Prep | Prep | |
| Impurity | RRT | #1 | #2 | Mean | RRT | #1 | #2 | Mean | RRT | #1 | #2 | Mean |
| BA 2 | 0.26 | 11.63 | 11.07 | 11.35 | | 8.92 | 8.65 | 8.79 | | 6.76 | 6.47 | 6.61 |
| 1 | | | | | | 0.21 | 0.21 | 0.21 | | 0.23 | 0.20 | 0.22 |
| 2 | | | | | | | | | | | 0.13 | 0.13 |
| 3 | 0.84 | ND | 0.21 | 0.21 | | | | | | | | |
| 4 | | | | | | 0.13 | 0.17 | 0.15 | | 0.44 | 0.56 | 0.50 |
| 5 | 0.66 | 0.16 | 0.20 | 0.18 | | 0.38 | 0.48 | 0.43 | | 0.66 | 0.79 | 0.73 |
| 6 | | | | | | | | | | 0.07 | 0.08 | 0.08 |
| 7 | | | | | | | | | | | | |
| 8 | | | | | | | 0.10 | 0.10 | | | | |
| 9 | 1.47 | ND | 0.12 | 0.12 | | | | | | | | |
| Unknown | 0.18 | 0.13 | 0.14 | 0.14 | 0.23 | 0.18 | 0.20 | 0.19 | 0.22 | 0.15 | 0.16 | 0.16 |
| | | | | | 0.43 | 0.06 | 0.05 | 0.06 | 0.41 | 0.05 | | 0.05 |
| | | | | | 0.47 | 0.06 | 0.06 | 0.06 | 0.43 | 0.06 | | 0.06 |
| | | | | | 0.53 | 0.08 | 0.08 | 0.08 | 0.46 | 0.06 | 0.06 | 0.06 |
| | | | | | 0.76 | 0.09 | 0.09 | 0.09 | 0.53 | 0.09 | 0.11 | 0.10 |
| | | | | | 0.89 | 0.12 | 0.38 | 0.25 | 0.62 | 0.05 | | 0.05 |
| | | | | | 1.40 | | 0.42 | 0.42 | 0.76 | 0.26 | 0.28 | 0.27 |
| | | | | | 1.44 | | 0.14 | 0.14 | 0.89 | 0.15 | 0.39 | 0.27 |
| | | | | | 1.47 | | 0.24 | 0.24 | 1.15 | | 0.07 | 0.07 |
| | | | | | | | | | 1.41 | 0.14 | 0.61 | 0.38 |
| | | | | | | | | | 1.45 | | 0.17 | 0.17 |
| | | | | | | | | | 1.49 | | 0.17 | 0.17 |
| Totals | | 0.29 | 0.67 | 0.65 | | 1.31 | 2.62 | 2.42 | | 2.41 | 3.78 | 3.47 |

TABLE 44C
Impurities for Betamethasone Dipropionate Cream Formulation (stored at 40° C./75% RH)

| | Initial | | | | 1 Month | | | | 3 Month | | | |
|---|---|---|---|---|---|---|---|---|---|---|---|---|
| | | Prep | Prep | | | Prep | Prep | | | Prep | Prep | |
| Impurity | RRT | #1 | #2 | Mean | RRT | #1 | #2 | Mean | RRT | #1 | #2 | Mean |
| BA 2 | 0.26 | 11.63 | 11.07 | 11.35 | | 7.13 | 6.89 | 7.01 | | 3.71 | 3.78 | 3.75 |
| 1 | | | | | | 0.25 | 0.24 | 0.25 | | 0.30 | 0.31 | 0.31 |
| 2 | | | | | | | | | | | 0.18 | 0.18 |
| 3 | 0.84 | ND | 0.21 | 0.21 | | | | | | | | |
| 4 | | | | | | 0.48 | 0.58 | 0.53 | | 1.86 | 2.12 | 1.99 |
| 5 | 0.66 | 0.16 | 0.20 | 0.18 | | 0.59 | 0.68 | 0.64 | | 0.86 | 0.99 | 0.93 |
| 6 | | | | | | | | | | 0.14 | 0.05 | 0.10 |
| 7 | | | | | | | 0.15 | 0.15 | | | 0.10 | 0.10 |
| 8 | | | | | | | 0.16 | 0.16 | | | | |
| 9 | 1.47 | ND | 0.12 | 0.12 | | | | | | | | |
| Unknown | 0.18 | 0.13 | 0.14 | 0.14 | 0.23 | 0.22 | 0.23 | 0.23 | 0.22 | 0.20 | 0.21 | 0.21 |
| | | | | | 0.41 | 0.05 | | 0.05 | 0.41 | 0.25 | 0.28 | 0.27 |
| | | | | | 0.43 | 0.05 | 0.06 | 0.06 | 0.43 | | 0.07 | 0.07 |
| | | | | | 0.47 | 0.06 | 0.06 | 0.06 | 0.46 | 0.07 | 0.10 | 0.09 |
| | | | | | 0.53 | 0.09 | 0.08 | 0.09 | 0.53 | 0.12 | 0.11 | 0.12 |
| | | | | | 0.76 | 0.29 | 0.30 | 0.30 | 0.58 | 0.11 | 0.10 | 0.11 |
| | | | | | 0.89 | 0.13 | 0.37 | 0.25 | 0.62 | 0.07 | 0.06 | 0.07 |
| | | | | | 1.40 | | 0.50 | 0.50 | 0.67 | 0.05 | | 0.05 |
| | | | | | 1.44 | | 0.15 | 0.15 | 0.76 | 0.84 | 0.87 | 0.86 |
| | | | | | 1.47 | | 0.21 | 0.21 | 0.89 | 0.17 | 0.51 | 0.34 |
| | | | | | | | | | 1.41 | 0.18 | 0.84 | 0.51 |
| | | | | | | | | | 1.45 | | 0.12 | 0.12 |
| | | | | | | | | | 1.49 | | 0.13 | 0.13 |
| Totals | | 0.29 | 0.67 | 0.65 | | 2.21 | 3.77 | 3.63 | | 5.22 | | 6.56 |

TABLE 45
Viscosity for Betamethasone Dipropionate Cream Formulation (cPs)

| | | | Initial | | 1 Month | | 3 Month | |
|---|---|---|---|---|---|---|---|---|
| Lot # | Speed | Prep. | Viscosity | Torque | Viscosity | Torque | Viscosity | Torque |
| DDI09Dec2020p28 | 3.00 | 1 | 19310 | 7.3 | 18520 | 7.0 | 18790 | 7.1 |
| Sample #1 | | 2 | 19310 | 7.3 | 18260 | 6.9 | 18520 | 7.0 |
| | | 3 | 19050 | 7.2 | 18260 | 6.9 | 18260 | 6.9 |
| | | Mean | 19223 | | 18347 | | 18523 | |
| | 30.00 | 1 | 3889 | 14.7 | 3810 | 14.4 | 3784 | 14.3 |
| | | 2 | 3889 | 14.7 | 3810 | 14.4 | 3784 | 14.3 |
| | | 3 | 3863 | 14.6 | 3810 | 14.4 | 3784 | 14.3 |
| | | Mean | 3880 | | 3810 | | 3784 | |
| | 250.00 | 1 | 997.0 | 31.4 | 981.1 | 30.9 | 981.1 | 30.9 |
| | | 2 | 997.0 | 31.4 | 981.1 | 30.9 | 977.9 | 30.8 |
| | | 3 | 997.0 | 31.4 | 981.1 | 30.9 | 977.9 | 30.8 |
| | | Mean | 997.0 | | 981.1 | | 979.0 | |
| DDI09Dec2020p28 | 3.00 | 1 | 19310 | 7.3 | 19580 | 7.4 | 19580 | 7.4 |
| Sample #2 | | 2 | 19310 | 7.3 | 19050 | 7.2 | 19050 | 7.2 |
| | | 3 | 19050 | 7.2 | 18790 | 7.1 | 18790 | 7.1 |
| | | Mean | 19223 | | 19140 | | 19140 | |
| | 30.00 | 1 | 3889 | 14.7 | 3837 | 14.5 | 3784 | 14.3 |
| | | 2 | 3889 | 14.7 | 3810 | 14.4 | 3757 | 14.2 |
| | | 3 | 3863 | 14.6 | 3784 | 14.3 | 3757 | 14.2 |
| | | Mean | 3880 | | 3810 | | 3766 | |
| | 250.00 | 1 | 997.0 | 31.4 | 977.9 | 30.8 | 962.0 | 30.3 |
| | | 2 | 997.0 | 31.4 | 977.9 | 30.8 | 955.7 | 30.1 |
| | | 3 | 997.0 | 31.4 | 974.7 | 30.7 | 952.5 | 30.0 |
| | | Mean | 997.0 | | 976.8 | | 956.7 | |
| DDI09Dec2020p28 | 3.00 | 1 | 19310 | 7.3 | 18790 | 7.1 | 19580 | 7.4 |
| Sample #3 | | 2 | 19310 | 7.3 | 18260 | 6.9 | 19050 | 7.2 |
| | | 3 | 19050 | 7.2 | 17990 | 6.8 | 18790 | 7.1 |
| | | Mean | 19223 | | 18347 | | 19140 | |
| | 30.00 | 1 | 3889 | 14.7 | 3678 | 13.9 | 3625 | 13.7 |
| | | 2 | 3889 | 14.7 | 3651 | 13.8 | 3625 | 13.7 |
| | | 3 | 3863 | 14.6 | 3625 | 13.7 | 3598 | 13.6 |
| | | Mean | 3880 | | 3651 | | 3616 | |
| | 250.00 | 1 | 997.0 | 31.4 | 965.2 | 30.4 | 943.0 | 29.7 |
| | | 2 | 997.0 | 31.4 | 958.9 | 30.2 | 936.6 | 29.5 |
| | | 3 | 997.0 | 31.4 | 962.0 | 30.3 | 933.5 | 29.4 |
| | | Mean | 997.0 | | 962.0 | | 937.7 | |

TABLE 46
Osmolality for Betamethasone Dipropionate
Cream Formulation (mOsm/kg)

| Lot # | Initial | 1 Month | 3 Months |
|---|---|---|---|
| DDI09Dec2020p28 | 345 | 346 | 344 |
| Sample #1 | | | |
| DDI09Dec2020p28 | 345 | 355 | 338 |
| Sample #2 | | | |
| DDI09Dec2020p28 | 345 | 343 | 336 |
| Sample #3 | | | |

### Example 17: Cream Formulations with Other Active Agents

The aqueous phase for a cream formulation of mometasone furoate monohydrate (lot #2021-06-03) was prepared by dispensing 144.82 g of water into a mixer with a 4-blade propeller at approximately half height followed by mixing at 200-300 rpm to dissolve 0.125 g of disodium EDTA and 4.37 g glycerin in the water. 1.502 g of Carbopol 980 was added by sprinkling a layer across the surface and pulsing the mixer 2-5×after each addition. The mixture was then mixed for 30 minutes at 800-1000 RPM with rotating the beaker ever 5-10 minutes. 43.08 g pf 1% NaOH was added while mixing at approximately 1000 RPM and the mixture QS with water to 250 g. While mixing at 200-300 RPM, 12.51 g of polysorbate 80 was added and mixed for approximately 45 minutes at about half height with “milk shake” mixing every 5-10 minutes.

The oil phase for a cream formulation of mometasone furoate monohydrate was prepared by adding 2.5 g of polyoxyl 40 stearate, 2.5 g of cetyl alcohol, 1.25 g of glyceryl monostearate, 20.00 g of petrolatum and 7.51 g of Span 20 to a beaker with a stir bar and heating the mixture to 65+/−5° C. with mixing for approximately 15 minutes until most solids were melted (settings: 80° C.; 100-350 RPM) followed by slow stirring for approximately 10 minutes until homogenous (settings: 75° C.; 50-100 RPM).

The aqueous phase was then mixed for approximately 5 minutes with a disk impeller blade and heated to 62+/−3° C. at the highest RPM that did not cause foaming (approximately 1200+RPM) and waiting for approximately 30 minutes for heating. 0.125 g of mometasone furoate monohydrate was added to each of the aqueous phase and the oil phase and each were then mixed for approximately 10-15 minutes.

The blade in the aqueous phase was adjusted to half height and mixed with high shear at approximately 1800 RPM and the oil phase was added to the hot aqueous phase and the mixture was removed from heat. The mixture was stirred for approximately 45 minutes with “milk shaking” every 5-10 minutes. 2.24 g of benzyl alcohol was then added to the combined mixture and mixed at high shear at approximately 1800 RPM, then stirred for approximately 30 minutes at approximately 1200 RPM with “milk shaking” every 5-10 minutes. pH was measured (pH was 5.958) and the mixture was QS for water loss with additional water and mixed for approximately 10 minutes with “milk shaking” every 3-5 minutes.

A placebo cream (lot #2020-11-01) was prepared similarly without mometasone furoate monohydrate using 0125 g disodium EDTA, 4.389 g of glycerin, 1.501 g Carbopol 980, 43.10 g of 1% NaOH, 12.46 g of polysorbate 80, 2.5 g of polyoxyl 40 stearate, 2.5 g of cetyl alcohol, 1.25 g of glyceryl monostearate, 20.00 g of petrolatum, 7.51 g of Span 20 and 2.26 g of benzyl alcohol. pH of the cream was 5.953.

A fluticasone propionate cream was prepared similarly using fluticasone propionate in place of mometasone furoate monohydrate, using 0.1253 g of disodium EDTA, 4.39 g of glycerin, 1.5009 g Carbopol 980, 43.06 g of 1% NaOH, 12.51 g of polysorbate 80, 2.5 g of polyoxyl 40 stearate, 2.5 g of cetyl alcohol, 1.25 g of glyceryl monostearate, 20.00 g of petrolatum, 7.5 of Span 20, 2.24 g of benzyl alcohol and 12.517 g of fluticasone propionate. This cream was assigned lot #2021-07-01. An additional lot was prepared using 0.100 g of disodium EDTA, 3.5 g of glycerin, 1.201 g Carbopol 980, 34.38 g of 1% NaOH, 10.01 g of polysorbate 80, 2 g of polyoxyl 40 stearate, 2 g of cetyl alcohol, 1 g of glyceryl monostearate, 16.00 g of petrolatum, 6 of Span 20, 1.81 g of benzyl alcohol and 100.1 g of fluticasone propionate to a total of 200 g. This cream was assigned lot #2021-07-02. The final pH of these two creams was 5.961 and 5.964, respectively.

Degradation and impurity analysis of the creams was performed using HPLC. The following parameters were used for the analysis of mometasone furoate monohydrate.

TABLE 47
HPLC Parameters

| Parameter: | Value | | |
|---|---|---|---|
| Column: | Kinetex ® 2.6 μm XB-C18 100 Á LC Column 50 × | | |
| | 4.6 mm (Phenomenex brand) | | |
| Mobile Phase A: | 30% Methanol | | |
| Mobile Phase B: | 80% Methanol | | |
| Gradient | Time (min) | % A | % B |
| | 0 | 55 | 45 |
| | 3 | 55 | 45 |
| | 4 | 25 | 75 |
| | 10.50 | 25 | 75 |
| | 10.51 | 45 | 55 |
| | 19 | 45 | 55 |

| | |
|---|---|
| Flow Rate: | 0.7 mL/min |
| Run Time: | 16 min |
| Column Temp: | 25° C. |
| Injection Volume: | 10 μL |
| Wavelength: | 248 nm for MFM (DAD detector used) |
| Concentration: | 250 μg/mL MFM and 5.8848 μg/mL Prednisone |
| Needle Wash: | 50% Methanol in water |
| Diluent: | 90% Methanol |

Neat mometasone furoate monohydrate (MFM) material was degraded and analyzed for potential chromatographic interference. Briefly, neat MFM was exposed to various conditions to induce degradation including heat (60° C./5 days), light (6 inches away from a white bench light for 5 days), acid (0.1N HCl for 1 hour followed by neutralization with 0.1N NaOH), base (0.1N NaOH for 1 hour followed by neutralization with 0.1N HCl), and oxidation (3% H₂O₂ for 1 hour). All samples including the degraded samples, control samples (dissolved in HPLC-grade methanol), and the reference solution were analyzed.

Percent recovery is provided in Table 48 below for each condition.

TABLE 48
Percent Recovery of MFM for Each Degradation Condition

| Degradation Method | Acid | Base | Peroxide | Heat | Light | Control |
|---|---|---|---|---|---|---|
| Actual Amount (μg/mL) | 240.289 | 6.091 | 262.576 | 249.211 | 249.916 | 254.279 |
| Theoretical Amount (μg/mL) | 251.550 | 252.10 | 266.950 | 253.450 | 252.70 | 255.60 |
| % Recovery | 95.52 | 2.42 | 98.36 | 98.33 | 98.90 | 99.48 |

Table 49 below provides the Relative Retention Times (RRTs) and detection ≥0.05% for the samples.

TABLE 49
Degradant Peak Table for MFM (degraded neat material)

| | RT | | | | | | | |
|---|---|---|---|---|---|---|---|---|
| | (min) | RRT with respect to MFM | | | | | | |
| Condition | MFM | 0.61 | 0.71 | 0.79 | 0.87* | 0.96* | 1.07 | 1.12* |
| 0.1N HC1 | 8.633 | detected | detected | detected | detected | detected | detected | detected |
| 0.1N NaOH | 8.633 | — | — | — | — | detected | detected | detected |
| 3% H₂O₂ | 8.637 | — | — | — | — | detected | — | — |
| 60° C. for 5 days | 8.693 | — | — | — | — | detected | — | — |
| White light for 5 days | 8.690 | — | — | — | — | — | — | — |
| 4° C. (Control) | 8.693 | — | — | — | — | detected | — | — |

≥0.05% Peak Area; detected above the 0.05% cutoff:
“—” indicates no peak:
*= related substance

MFM neat and degraded materials were spiked into placebo cream (lot #2020-11-01) to investigate potential partitioning effects and peak interferences and to compare spiked and active cream samples by the procedure in Table 50 with the exception that for acid, base and peroxide degradations, the additions of HCl, NaOH and peroxide were calculated accordingly.

TABLE 50
Extraction Procedure

| Step | Description |
|---|---|
| 1 | Accurately weigh 2 g (±0.2 g) into a 50 mL centrifuge tube, |
| | spreading the sample around the walls of the tube |
| 2 | Use volumetric pipettes to transfer 6.00 mL of methanol (HPLC |
| | grade) and 1.20 mL of internal standard solution (prednisone in |
| | methanol) and 0.80 mL H₂O to the tube (results in 8 mL solvent |
| | mixture, 10% H₂O |
| 3 | Vortex the tube for ~30 seconds, or until the sample is dispersed |
| 4 | Place the tube in a 70° C. oven for 15 minutes to melt and/or |
| | dissolve the sample matrix |
| 5 | Remove the tube from heat and immediately vortex mix for ≥30 |
| | seconds to disperse the sample |
| 6 | Place the tube on a shaker at 400 rpm for 20 minutes as the |
| | solution cools |
| 7 | Place the tube in a freezer (≤20° C.) for 15 minutes to help |
| | solidify non-aqueous phase |
| 8 | Centrifuge the tube at 3000 G and 4° C. for 30 minutes |
| 9 | Filter the supernatant with a syringe filter (Thermo Fisher, 17 mm, |
| | 0.2 μm pore size) and then transfer to an HPLC vile for analysis |

Percent recovery of Force-Degraded Materials Spiked into Placebo is provided in Table 51 below while recovery of active cream formulation (0.1% MFM) is provided in Table 52 below. Total MFM-Related Peak Areas are provided in Table 53 below.

TABLE 51
Recovery of Force-Degraded Materials Spiked into Placebo

| | | % Recovery of MFM | | Difference |
|---|---|---|---|---|
| | | Neat | Spiked | with |
| Material | Conditions | Material | Placebo | Placebo |
| Mometasone | White light (5 days) | 98.90% | 93.61% | 5.29% |
| Furoate | 60° C. (5 days) | 98.33% | 93.37% | 4.96% |
| Momohydrate | 0.1N HC1 (1 hour) | 95.52% | 90.29% | 5.23% |
| | 0.1N NaOH (1 | 2.42% | 2.29% | 0.13% |
| | hour) | | | |
| | 3% H₂O₂ (1 | 98.36% | 93.16% | 5.20% |
| | hour) | | | |
| | Control (neat MFM) | 99.48% | 93.61% | 5.87% |

TABLE 52
Recovery of Active Cream Formulation (0.1% MFM)

| | | Recovery | | |
|---|---|---|---|---|
| Material | Storage Conditions | of MFM | Placebo | Difference |
| Active cream | Kept at room | 84.02% | 0% | 84.02% |
| (0.1% MFM) | temperature in the dark | | | |

TABLE 53
Total MFM-Related Peak Areas
Peak Areas of MFM and Relative Peak Areas of Degradants

| | | Peak Areas for Peaks of Interest | | | | | | | | Total |
|---|---|---|---|---|---|---|---|---|---|---|
| | | (excluding placebo, IS, and excipient-related peaks), RRTs with respect to MFM | | | | | | | | MFM- |
| | | | RRT = | RRT = | RRT = | RRT = | RRT = | RRT = | RRT = | Related |
| Sample Info | | MFM | 0.61 | 0.71 | 0.79 | 0.87 | 0.96 | 1.07 | 1.12 | Area |
| Active Cream | | 114.765 | no peaks | no peaks | no peaks | no peaks | 0.085 | no peaks | no peaks | 114.85 |
| Spiked | Control | 125.120 | no peaks | no peaks | no peaks | no peaks | 0.171 | no peaks | no peaks | 125.291 |
| | Acid | 116.720 | no peaks | no peaks | no peaks | no peaks | 2.964 | 0.05 | 0.353 | 120.087 |
| | Base | 2.991 | 0.097 | 0.143 | 0.433 | 5.187 | 14.967 | 55.374 | 10.016 | 89.208 |
| | Peroxide | 128.187 | no peaks | no peaks | no peaks | no peaks | 0.176 | no peaks | no peaks | 128.363 |
| | Heat | 122.536 | no peaks | no peaks | no peaks | no peaks | 0.161 | no peaks | no peaks | 122.697 |
| | Light | 123.671 | no peaks | no peaks | no peaks | no peaks | 0.152 | no peaks | no peaks | 123.823 |

These results show that there is no apparent degradation of MFM due to the cream making process as the common peak at RRT=0.96 (with respect to MFM) was identified during the forced degradation analyses.

A similar analysis was performed of the 0.005% fluticasone propionate cream (lot #2021-07-01). HPLC parameters are provided in Table 54.

TABLE 54
HPLC Parameters

| Parameter: | Initial Value | | | |
|---|---|---|---|---|
| Column: | Kinetex ® 2.6 μm XB-C18 100 Á LC Column 50 × | | | |
| | 4.6 mm (Phenomenex brand) | | | |
| Mobile Phase A: | Methanol | | | |
| Mobile Phase B: | Acetonitrile | | | |
| Mobile Phase C: | Monobasic ammonium phosphate//H₃PO₄ buffer at | | | |
| | pH = 3.5 +/− 0.03 | | | |
| Isocratic | Time (min) | A % | B % | C % |
| | 0-20 (min) | 46 | 5 | 49 |

| | |
|---|---|
| Flow Rate: | 0.9 mL/min |
| Run Time: | 20-32 min |
| Column Temp: | 50° C. |
| Injection Volume: | 10 μL |
| Wavelength: | 240 nm for MFM (DAD detector used) |
| Concentration: | 15 μg/mL FP and 9.808 μg/mL Prednisone |
| Needle Wash: | 50% Methanol in water |
| Diluent: | 90% Methanol |

Percent Recovery for fluticasone propionate for each forced degradation condition is provided in Table 55 below.

TABLE 55
Percent Recovery of Fluticasone Propionate for Each Forced Degrdation Condition

| Degradation Method | Acid | Base | Peroxide | Heat | Light | Control |
|---|---|---|---|---|---|---|
| Actual Amount (μg/mL) | 12.1791 | 11.7484 | 16.4961 | 12.0739 | 11.6609 | 12.5695 |
| Theoretical Amount (μg/mL) | 12.4997 | 12.5000 | 17.8514 | 12.4998 | 12.4999 | 12.4999 |
| % Recovery | 97.44 | 93.99 | 92.41 | 96.59 | 93.29 | 100.6 |

The extraction procedure is provided in Table 56 below.

TABLE 56
Extraction Procedure

| Step | Description |
|---|---|
| 1 | Accurately weigh 1 g (±0.1 g) into a 50 mL centrifuge tube, |
| | spreading the sample around the walls of the tube |
| 2 | Use eppendorf pipettes to transfer 6.94473 mL of methanol (HPLC |
| | grade) and 255.27 uL of internal standard solution (prednisone |
| | in methanol) |
| 3 | Vortex the tube for ~30 seconds, or until the sample is |
| | dispersed and sonicate for 10 minutes |
| 4 | Add 0.80 mL H₂O to the tube (results in 8 mL solvent mixture. |
| | 10% H₂O) |
| 5 | Vortex again for 30 seconds |
| 6 | Place the tube in a 70° C. oven for 15 minutes to melt and/ |
| | or dissolve the sample matrix |
| 7 | Remove the tube from heat and immediately vortex mix for ≥30 |
| | seconds to disperse the sample |
| 8 | Sonicate the samples for 10 minutes |
| 9 | Place the tube on a shaker at 400 rpm for 20 minutes as the |
| | solution cools |
| 10 | Place the tube in a freezer (≤20° C.) for 15 minutes to |
| | help solidify non-aqueous phase |
| 11 | Centrifuge the tube at 3000 G and 4° C. for 30 minutes |
| 12 | Filter the supernatant with a syringe filter (Thermo Fisher, |
| | 17 mm, 0.2 μm pore size) and then transfer to an HPLC vial |
| | for analysis |

The Percent Recovery of Force-Degraded Materials Spiked into Placebo is provided in Table 57 below. The Percent Recovery of Active Cream Formulations with Fluticasone Propionate is provided in Table 58 below. The Total Fluticasone Propionate-Related Peak Areas are provided in Table 59 below.

TABLE 57
Percent Recovery of Force-Degraded Materials Spiked into Placebo

| | | % Recovery of | | |
|---|---|---|---|---|
| | | Degraded Material | | Difference |
| | | Neat | Spiked | with |
| Material | Conditions | Material | Placebo | Placebo |
| Fluticasone | 0.1N HC1 (1 hour) | 97.44% | 91.41% | 6.03% |
| Propionate | 0.1N NaOH (1 | 93.99% | 93.80% | 0.19% |
| 0.005% | hour) | | | |
| | 3% H₂O₂ (1 hour) | 92.41% | 96.44% | 4.03% |
| | 60° C. (3 days) | 96.59% | 97.68% | 1.09% |
| | White light (3 days) | 93.29% | 96.18% | 2.71% |
| | Control (undegraded | 100.6% | 103.31% | 0.2-1.1% |
| | FP, kept at 4 C.) | | | |

TABLE 58
Percent Recovery of Active Cream Formulation
with Fluticasone Propionate

| | | Recovery | | |
|---|---|---|---|---|
| Material | Storage Conditions | of FP | Placebo | Difference |
| Active cream | Kept at room | 86.27% | 0% | 86.27% |
| with 0.005% FP | temperature in | | | |
| by formulation | the dark | | | |

TABLE 59
Total Fluticasone Propionate-Related Peak Areas
Peak Areas of MFM and Relative Peak Areas of Degradants

| | | Peak Areas for Peaks of Interest | | | | Total |
|---|---|---|---|---|---|---|
| | | (excluding placebo, IS, and excipient-related peaks) | | | | MFM- |
| | | | RRT = | RRT = | RRT = | Related |
| Sample Info | | FP | 0.32 | 0.33 | 1.17 | Area |
| Active Cream | | 2.399 | 0.029 | no peak | 0.176 | 1.604 |
| Spiked | Control | 2.607 | no peak | 0.012 | 0.046 | 2.665 |
| | Acid | 2.377 | 0.033 | no peak | 0.221 | 2.631 |
| | Base | 2.395 | 0.032 | no peak | 0.166 | 2.593 |
| | Peroxide | 2.424 | no peak | no peak | no peak | 2.424 |
| | Heat | 2.924 | no peak | no peak | 0.183 | 3.107 |
| | Light | 2.599 | | no peak | 0.159 | 2.791 |

Since FP peak is relatively small, degradant peaks above 0.025% were included.

Except for peroxide-treated fluticasone propionate, all spiked and active cream extractions resulted in a common peak at RRT=1.17 (with respect to fluticasone propionate) which was not identified in the forced degradation or neat chromatograms and is not a related substance or true degradant which indicates there could be degradation caused by the extraction method for the active cream, the base addition, heating the cream or light exposure which is supported by the peak present at RRT=0.32 which is also present in the spiked acid, base, and light-treated forced degradation samples.

For all the foregoing HPLC in this example, prednisone was used as an internal standard.

### Example 18: D-Values for Betamethasone Dipropionate Cream Formulation

The D-value, which measures the autoclave time at a specific temperature to kill 90% of a bacterial reference—in this case, Bacillus substilis “S230”—was determined for 0.05% betamethasone dipropionate cream as prepared in Example 8. Autoclave temperatures used included 110° C., 115° C. and 121° C. for times ranging from 0 to 8 minutes for 110° C., from 0 to 4 minutes for 115° C., and from 0 to 3 minutes for 121° C.

Exposure data is provided in Table 60 below.

TABLE 60
Exposure Data

| | |
|---|---|
| Exposure Time (minutes) | Population |

110° C.

| | |
|---|---|
| 0 | 6.4375 × 10⁶ |
| 2.0 | 2.6563 × 10⁵ |
| 4.0 | 5.5688 × 10⁴ |
| 6.0 | 2.5000 × 10² |
| 8.0 | 1.6094 × 10² |

115° C.

| | |
|---|---|
| 0 | 2.8625 × 10⁶ |
| 1.0 | 2.6250 × 10⁶ |
| 2.0 | 1.8875 × 10⁵ |
| 3.0 | 4.3594 × 10² |
| 4.0 | 9.6875 × 10¹ |

121° C.

| | |
|---|---|
| 0 | 2.8625 × 10⁶ |
| 0.5 | 1.5688 × 10⁶ |
| 1.0 | 3.3563 × 10⁵ |
| 1.5 | 2.4000 × 10⁴ |
| 2.0 | 1.1500 × 10⁴ |
| 2.5 | 8.2813 × 10¹ |
| 3.0 | 6.5625 × 10¹ |

In 3.0 mL syringes, the Duo value was found to be 1.6 minutes based on a line of best fit of the survivor curve, while the D115 value was found to be 0.8 minutes and the D121 value was found to be 0.6 minutes.

All references, including publications, patent applications, and patents, cited herein are hereby incorporated by reference to the same extent as if each reference were individually and specifically indicated to be incorporated by reference and were set forth in its entirety herein.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising:
a tonicity agent;
a therapeutic active agent; and
an emulsifier,
wherein the composition is a cream, and
wherein the composition has an osmolality of about 270 mOsm/kg to about 360 mOsm/kg.

2. (canceled)

3. The composition of claim 1, wherein the tonicity agent is selected from the group consisting of glycerin, propylene glycol, polyethylene glycol, butylene glycol, cyclomethicone, polydextrose, sodium hyaluronate, sodium lactate, sorbitol, trehalose, triacetin, xylitol, sodium chloride, potassium chloride and combinations thereof.

4. The composition of claim 1, wherein the tonicity agent is glycerin.

5. The composition of claim 1, wherein the composition does not include propylene glycol.

6. (canceled)

7. (canceled)

8. The composition of claim 1, wherein the tonicity agent is present in the composition at about 1% (w/w) to about 5% (w/w) based on the total weight of the composition.

9. (canceled)

10. (canceled)

11. (canceled)

12. (canceled)

13. (canceled)

14. The composition of any one of claim 18, wherein the emulsifier is a combination of a polyoxyethylene sorbitan fatty acid ester, a polyoxyethylene stearate, cetyl alcohol, and sorbitan monolaurate.

15. (canceled)

16. The composition of claim 14, wherein the emulsifier is present in the composition at about 5% (w/w) to about 115% (w/w) based on the total weight of the composition.

17. (canceled)

18. (canceled)

19. (canceled)

20. The composition of claim 1, wherein the emulsifier is present in the composition at about 5% (w/w) to about 15% (w/w) based on the total weight of the composition.

21.-37. (canceled)

38. The composition of claim 1, wherein pH of the composition is selected from the group consisting of 5, 5.1, 5.2, 5.3, 5.4, 5.5, 5.6, 5.7, 5.8, 5.9, 6, 6.1, 6.2, 6.3, 6.4, and 6.5.

39. The composition of claim 1, further comprising a tonicity modifier.

40. (canceled)

41. The composition of claim 39, wherein the tonicity modifier is benzyl alcohol.

42. The composition of claim 39, wherein the tonicity modifier is present in the composition at about 0.5% (w/w) to about 1.5% (w/w) based on the total weight of the composition.

43.-51. (canceled)

52. The composition of claim 1, wherein the therapeutic active agent comprises a steroid.

53. (canceled)

54. (canceled)

55. The composition of claim 52, wherein the steroid is betamethasone dipropionate.

56. The composition of claim 55, wherein the steroid is present in the composition at from about 0.03% (w/w) to about 0.3% (w/w) based on the total weight of the composition.

57.-64. (canceled)

65. The composition of claim 1, wherein the composition further comprises a stabilizing agent.

66. (canceled)

67. The composition of claim 65, wherein the stabilizing agent is edetic acid or a pharmaceutically acceptable salt thereof.

68. The composition of claim 65, wherein the stabilizing agent is disodium edetate.

69. The composition of claim 68, wherein the stabilizing agent is present in the composition at about 0.005% (w/w) to 0.25% (w/w) based on the total weight of the composition.

70. (canceled)

71. The composition of any claim 1, wherein the composition has a viscosity as measured by a Brookfield RVDVII+ with Spindle 28 at room temperature of (1) from about 200,000 centipoise (cPs) to about 2,000,000 cPs at a shear rate of about 0.3 RPM; (2) from about 100,000 cPs to about 1,500,000 cPs at a shear rate of about 0.5 RPM; (3) from about 100,000 cPs to about 1,000,000 at a shear rate of about 0.6 RPM; (4) from about 50,000 cPs to 800,000 cPs at a shear rate of about 0.8 RPM; (5) from about 50,000 cPs to about 750,000 cPs at a shear rate of about 1 RPM; (6) from about 40,000 cPs to about 500,000 cPs at a shear rate of about 1.5 RPM; (7) from about 30,000 cPs to about 250,000 cPs at a shear rate of about 2.0 RPM; (8) from about 20,000 cPs to about 200,000 cPs at a shear rate of about 2.5 RPM; (9) from about 20,000 cPs to about 200,000 cPs at a shear rate of about 3.0 RPM; (10) from about 15,000 cPs to about 150,000 cPs at a shear rate of about 4.0 RPM; (11) from about 15,000 cPs to about 150,000 cPs at a shear rate of about 5.0 RPM; (12) from about 10,000 cPs to about 100,000 cPs at a shear rate of about 6.0 RPM; (13) about 8,000 cPs to about 70,000 cPs at a shear rate of about 10.0 RPM; (14) from about 10,000 cPs to about 60,000 cPs at a shear rate of about 12.0 RPM; (15) from about 1,000 cPs to about 40,000 cPs at a shear rate of about 20.0 RPM; (16) from about 1,000 cPs to about 20,000 cPs at a shear rate of about 30.0 RPM; (17) from about 500 cPs to about 15,000 cPs at a shear rate of about 50.0 RPM; (18) from about 500 cPs to about 10,000 cPs at a shear rate of about 60.0 RPM; or (19) from about 250 cPs to about 7,000 cPs at a shear rate of about 100.0 RPM.

72.-75. (canceled)

76. The composition of claim 1, wherein the composition is an oil-in-water emulsion.

77. The composition of claim 76, wherein the composition has an oil globule size of less than 5 μm, 4 μm, 3 μm, 2 μm or 1 μm by number or volume mean.

78. (canceled)

79. The composition of claim 1, wherein the composition does not agglomerate, cream, sediment, flocculate, phase invert, coalesce or a combination thereof after storage at 25° C./60% Relative Humidity for 1 month, 3 months, 6 months, 12 months, 18 months, or 24 months.

80.-91. (canceled)

92. The composition of claim 1, wherein the composition is sterile.

93.-234. (canceled)

235. A method for treating a subject with a disease or condition associated with a nasal, sinonasal or nasopharyngeal tissue, comprising:
administering a composition of claim 1 to a sinonasal, nasal or nasopharyngeal tissue, wherein the therapeutic active agent of the composition is a steroid.

236. The method of claim 235, wherein the amount of the composition administered per tissue is from about 0.5 grams to about 5 grams.

237. The method of claim 235, wherein said disease or condition is selected from the group consisting of sinus edema, acute sinusitis, acute sinusitis infection, acute sinusitis bacterial infection, acute sinusitis viral infection, acute rhinosinusitis, ageusia, allergic fungal sinusitis, anosmia, bacterial sinusitis, barosinusitis, barotrauma, chronic polyposis, chronic bacterial sinusitis, chronic allergic fungal sinusitis, chronic sinusitis, chronic recurrent sinusitis, chronic recurrent sinusitis infection, chronic recurrent sinusitis bacterial infection, chronic recurrent sinusitis viral infection, chronic rhinosinusitis, chronic rhinosinusitis with polyps, chronic rhinosinusitis without polyps, chronic recurrent rhinosinusitis, central compartment atopic disease, cystic fibrosis, diffuse sinusitis, diffuse type 2 sinusitis, eosinophilic rhinosinusitis, fungal sinusitis, granulomatosis with polyangiitis, maxillary sinus infection, mucormycosis, nasal polyps, non-eosinophilic rhinosinusitis, non-eosinophilic chronic rhinosinusitis, paranasal sinus retention cysts, polymicrobial sinusitis, recurrent rhinosinusitis, recurrent acute rhinosinusitis, rhinosinusitis, sinusitis, sinonasal polyps, and sphenoid sinus infection.

238. (canceled)

239. (canceled)

240. The method of claim 235, wherein the step of administering is performed not more than one time on the subject.

241. (canceled)

242. (canceled)

243. (canceled)

244. The method of claim 235, wherein the step of administering is performed not more than once in a 30 day period.

245. (canceled)

246. (canceled)

247. The method of claim 235, wherein the step of administering is performed not more than once in a 90 day period.

248.-261. (canceled)

262. The method of claim 235, wherein the sinonasal or nasopharyngeal tissue is selected from the group consisting of maxillary sinus, frontal sinus, ethmoid sinus, sphenoid sinus, maxillary mucosa, frontal mucosa, ethmoid mucosa, sphenoid mucosa, turbinates, nasal passage, nasolacrimal duct, nasal cavity and nasal tissue.

263. (canceled)

264. (canceled)

265. (canceled)

266. A method for treating a subject with a disease or condition associated with an otic tissue, comprising:
administering a composition of claim 1 to an otic tissue, wherein the therapeutic active agent of the composition is a steroid.

267. The method of claim 266, wherein the total amount of the composition administered is from about 0.17 grams to about 2.1 grams.

268. The method of claim 266, wherein said disease or condition is selected from the group consisting of acute otitis media, acute localized external otitis (furunculosis), acute mastoiditis, acoustic neuroma, auditory processing disorder, autoimmune inner ear disease, benign paroxysmal positional vertigo, barotrauma, choleasteatoma, chronic external otitis, chronic otitis media, chronic otitis media with effusion, dizziness, erysipelas, herpes zoster otitis, hearing loss, infectious myringitis, inner ear infection, inner ear related vertigo, labyrinthitis, malignant otitis externa, Meniere's disease, middle ear infection, otitis media, otitis media with effusion, otitis media with perforation, otitis externa, otomycosis, outer ear infection, perforated eardrum, perichondritis, recurrent vestibulopathy, serous otitis media, superior semicircular canal dehiscence syndrome, tinnitus, tube otorrhea, vertigo, vestibulopathy, vestibular neuritis, and viral labyrinthitis.

269. The method of claim 266, wherein the step of administering is performed not more than one time on the subject.

270. (canceled)

271. (canceled)

272. (canceled)

273. The method of claim 266, wherein the step of administering is performed not more than once in a 30 day period.

274. (canceled)

275. (canceled)

276. The method of claim 266, wherein the step of administering is performed not more than once in a 90 day period.

277.-285. (canceled)

286. The method of claim 266, wherein the otic tissue is selected from the group consisting of the auricle, cochlea, ear canal, Eustachian tube, external auditory canal, inner ear, middle ear, outer ear, round window, semicircular canals, tympanic membrane, tympanic cavity, meatal tissue or hair cells.

287. The method of claim 266, wherein the step of administering the composition includes delivering about 0.01 mg to about 250 mg of steroid.

288-306. (canceled)
